(19) United States
(12) Patent Application Publication
(10) Pub. No.: US 2022/0040201 A1
(43) Pub. Date: Feb. 10, 2022

(54) LIQUID POLYMER DELIVERY SYSTEM FOR EXTENDED ADMINISTRATION OF DRUGS

(71) Applicant: TOLMAR INTERNATIONAL, LTD., Dublin 2 (IE)

(72) Inventors: Amy Haller VAN HOVE, Fort Collins, CO (US); Garrett Shane GLOVER, Fort Collins, CO (US); John Charles MIDDLETON, Fort Collins, CO (US); Avinash NANGIA, Fort Collins, CO (US)

(21) Appl. No.: 17/278,930

(22) PCT Filed: Sep. 24, 2019
(86) PCT No.: PCT/IB2019/001056

§ 371 Date: Mar. 23, 2021

Related U.S. Application Data
(60) Provisional application No. 62/736,182, filed on Sep. 25, 2018.

(51) Int. Cl. A61K 31/568; A61K 9/00; A61K 47/34; A61K 47/22; A61K 47/10; A61K 47/14

(52) U.S. Cl. CPC A61K 31/568; A61K 9/0019; A61K 47/14; A61K 47/22; A61K 47/10; A61K 47/34

(57) ABSTRACT

Liquid polymer pharmaceutical compositions comprising a biodegradable liquid polymer, a biocompatible solvent system, and an active pharmaceutical ingredient (API) are disclosed. The compositions of the invention are useful for providing extended, long-term release of the API.

### CROSS-REFERENCE TO RELATED APPLICATIONS

This application claims the benefit of priority under 35 U.S.C. § 119(e) to U.S. Provisional Patent Application 62/736,182, filed 25 Sep. 2018, the entirety of which is incorporated herein by reference.

### FIELD OF THE INVENTION

This application pertains to the field of biodegradable liquid polymer compositions that are administered into the body with syringes or needles and that are utilized to deliver a drug into the body over an extended period of time.

### BACKGROUND OF THE INVENTION

Biodegradable polymers are well known for their use in biomedical applications, such as sutures, surgical clips, staples, implants, and drug delivery systems. Such polymers include polyglycolides, polylactides, polycaprolactones, polyanhydrides, polyorthoesters, polydioxanones, polyacetals, polyesteramides, polyamides, polyurethanes, polycarbonates, polyphosphazenes, polyketals, polyhydroxybutyrates, polyhydroxyvalerates, polyhyaluronic acid, and polyalkylene oxalates.

Initially, biodegradable polymers were solid materials that were used to form solid articles such as sutures, staples, surgical clips, implants or microcapsules and microparticles. Because the polymers were solids, all of their applications in the biomedical field required that the polymeric structures be formed outside the body, and then inserted into the body for their use.

U.S. Pat. No. 5,278,201 to Dunn et al. (the “201 patent”) overcame the administration problems with the solid implants by dissolving the solid biodegradable polymers in a biocompatible solvent and injecting the solution into the body using standard syringes and needles, where the polymer in the solution precipitates or coagulates upon contact with aqueous body fluid to form a solid implant matrix. However, there remained several disadvantages with this in situ forming solid polymer system. Because the polymers used had relatively high molecular weights, the polymer solutions formed from the combination of the solid polymers and the biocompatible solvents were quite viscous, and administration required large bore needles and considerable injection force, were not easily injected into muscle tissue, and the solid implants formed from these polymer solutions tended to cause local irritation of the muscular tissue. For this reason, the foregoing polymer solutions were normally injected subcutaneously where the material would form quite distinct and noticeable bumps. Efforts by others to produce polymeric delivery systems that did not include a solvent, or were formed using non-polymeric materials, again suffered from viscosities unsuitable for injection, or were not suitable for a variety of extended release uses.

U.S. Pat. No. 8,187,640 to Dunn (the “640 patent”) addressed and solved problems associated with the solid implants of the '201 patent, by disclosing solution compositions of a biodegradable liquid polymer combined with a biocompatible solvent, which solvent would dissipate when the liquid polymer/solvent compositions were placed in a body, thereby forming a viscous liquid polymer material in the form of a film, a coating, a plug or other mass. The viscous liquid polymer material does not solidify upon injection into the body, but rather remains in situ in a viscous liquid form and, when combined with a drug, provides both an initial burst and extended release of the drug. The '640 patent disclosed that the rate of release of a drug from the in situ viscous liquid material can be controlled by altering the composition of the biodegradable polymer. According to the '640 patent, the composition of the liquid polymer, i.e., the type of monomer used or the ratio of monomers for copolymers or terpolymers, the end groups on the polymer chains, and the molecular weight of the polymer, determines the hydrophilicity or lipophilicity of the polymer material, as well as the degradation time of the liquid polymer implant. The '640 patent disclosed that, for faster release rates and shorter durations of release, more hydrophilic polymers can be used. For slower release of drug and longer duration of release, more hydrophobic polymer can be used. The '640 patent does not describe suitable variations of the end groups of the polymer chains. However, in the Examples section, this patent discloses the use of an alcohol, dodecanol, as an initiator, which results in a hydroxy group at the end of the polymer chain.

PCT Publication No. WO2017024027 describes the making of the low viscosity liquid polymeric delivery system as disclosed in the '640 patent to determine the rate and duration of release of drugs following subcutaneous administration of the drug-loaded delivery system. It was determined that the delivery system of the '640 patent is not suitable for long-term extended delivery of drugs beyond, e.g., 14 days. PCT Publication No. WO2017024027 discloses a different liquid polymer composition than that described in the '640 patent, which provided a markedly improved extended release of drugs as compared to the '640 patent. The liquid polymer composition described in PCT Publication No. WO2017024027 included a biodegradable liquid polymer with at least one carboxylic acid end group, and a ratio of monomer units to carboxylic acid end groups between about 5:1 and about 90:1.

Certain active pharmaceutical ingredients (APIs) have relatively low solubility in aqueous media, and/or are relatively hydrophobic (i.e. relatively low hydrophilicity). Such APIs may be more difficult to solubilize and/or may remain in solid form (in suspension) in various solvent systems as compared to APIs having lesser hydrophobicity/greater hydrophilicity. The use of such APIs in a liquid polymer composition can present special challenges in that it is desirable to maintain a stable form of the API throughout manufacturing, storage, and administration conditions, which may involve exposure to a wide range of temperatures during these processes. In addition, it would be desirable to be able modify the components of a liquid polymer composition containing such APIs in order to tailor the rate and duration of release of the API from the composition according to a particular target application. Therefore, there is a need in the art for liquid polymer compositions for use with APIs having relatively low solubility in aqueous media and/or relatively high hydrophobicity (i.e., relatively low hydrophilicity), where such liquid polymer compositions are stable over a wide range of temperatures and can be modified to control release of the API in order to provide a suitable extended release formulation for a target application.

### SUMMARY OF THE INVENTION

It is one aspect of the present invention to provide a pharmaceutical composition having an active pharmaceutical ingredient in suspension, comprising an active pharmaceutical ingredient having a log P of at least about 0; a biocompatible solvent or combination or mixture of solvents and/or co-solvents; and a biodegradable liquid polymer having a weight average molecular weight between about 1 kDa and about 25 kDa, wherein the active pharmaceutical ingredient is in substantially solid form in the polymer and solvent or combination or mixture of solvents and/or co-solvents at body temperature, and wherein the active pharmaceutical ingredient has a D_{v,50} of between about 1 μm and about 250 μm and a particle size span of between about 1 and about 8.

It is another aspect of the present invention to provide a pharmaceutical composition having an active pharmaceutical ingredient in suspension, comprising an active pharmaceutical ingredient having a log P of at least about 0; a biocompatible solvent or combination or mixture of solvents and/or co-solvents; and a biodegradable liquid polymer, wherein the active pharmaceutical ingredient is substantially in solid form in the polymer and solvent or combination or mixture of solvents and/or co-solvents at temperatures up to between about body temperature and at least about 45° C.

It is another aspect of the present invention to provide a pharmaceutical composition having an active pharmaceutical ingredient in suspension, comprising an active pharmaceutical ingredient having a log P of at least about 0; a biocompatible solvent or combination or mixture of solvents and/or co-solvents; and a biodegradable liquid polymer having a weight average molecular weight between about 1 kDa and about 25 kDa, wherein the active pharmaceutical ingredient has a D_{v,50} of between about 1 μm and about 250 μm and a particle size span of between about 1 and about 8.

In embodiments, the active pharmaceutical ingredient may be in substantially solid form in the polymer and solvent or combination or mixture of solvents and/or co-solvents at temperatures up to about 38° C., or up to about 40° C.

In embodiments, the active pharmaceutical ingredient may be in substantially solid form in the polymer and solvent or combination or mixture of solvents and/or co-solvents at temperatures up to at least about 45° C.

In embodiments, the active pharmaceutical ingredient may be in substantially solid form in the polymer and solvent or combination or mixture of solvents and/or co-solvents at temperatures between about 2° C. and about 38° C.

In embodiments, the active pharmaceutical ingredient may be in substantially solid form in the polymer and solvent or combination or mixture of solvents and/or co-solvents at temperatures between about 2° C. and about 45° C.

In embodiments, the log P of the active pharmaceutical ingredient may be at least about 2.5, or at least about 5.

In embodiments, prior to addition to the composition, the active pharmaceutical ingredient may have a D_{v,50} of between about 15 μm and about 200 μm and a particle size span of between about 1 and about 8.

In embodiments, in the final composition, the active pharmaceutical ingredient may have a D_{v,50} of between about 15 μm and about 200 μm and a particle size span of between about 1 and about 8.

In embodiments, prior to addition to the composition, or in the final composition, the active pharmaceutical ingredient may have a D_{v,50} of between about 15 μm and about 150 μm, or between about 50 μm and about 150 μm, or between about 50 μm and about 100 μm, or between about 50 μm and about 90 μm, or between about 65 μm and about 90 μm.

In embodiments, the active pharmaceutical ingredient may have a particle size span of between about 2 and about 6, or between about 2 and about 5, or between about 2 and about 4.

In embodiments, the biocompatible solvent or combination or mixture of solvents and/or co-solvents may be selected from the group consisting of acetone, butyrolactone, ε-caprolactone, N-cycylohexyl-2-pyrrolidone, diethylene glycol monomethyl ether, dimethyl acetamide, dimethyl formamide, dimethyl sulfoxide (DMSO), ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, N-methyl-2-pyrrolidone (NMP), low-molecular weight (MW) polyethylene glycol (PEG), polysorbate 80, polysorbate 60, polysorbate 40, polysorbate 20, polyoxyl 35 hydrogenated castor oil, polyoxyl 40 hydrogenated castor oil, sorbitan monolaurate, sorbitan monostearate, sorbitan monooleate, benzyl alcohol, n-propanol, isopropanol, tert-butanol, propylene glycol, 2-pyrrolidone, α-tocopherol, triacetin, tributyl citrate, acetyl tributyl citrate, acetyl triethyl citrate, triethyl citrate, esters thereof, and combinations and mixtures thereof.

In embodiments the biocompatible solvent or combination or mixture of solvents and/or co-solvents comprises a biocompatible solvent in combination with low-molecular weight (MW) polyethylene glycol (PEG).

In embodiments, the biocompatible solvent or combination or mixture of solvents and/or co-solvents may be selected from the group consisting of: dimethyl sulfoxide (DMSO), N-methyl-2-pyrrolidone (NMP), low-molecular weight (MW) polyethylene glycol (PEG), and combinations and mixtures thereof.

In embodiments, the biocompatible solvent or combination or mixture of solvents and/or co-solvents may be selected from the group consisting of: dimethyl sulfoxide (DMSO) in combination with low-molecular weight (MW) polyethylene glycol (PEG), and N-methyl-2-pyrrolidone (NMP) in combination with low-molecular weight (MW) polyethylene glycol (PEG).

In embodiments, the biocompatible solvent or combination or mixture of solvents and/or co-solvents may be N-methyl-2-pyrrolidone (NMP) in combination with polyethylene glycol (PEG) 300.

In embodiments, the biocompatible solvent or combination or mixture of solvents and/or co-solvents may be dimethyl sulfoxide (DMSO) in combination with polyethylene glycol (PEG) 400.

In embodiments, the active pharmaceutical ingredient may be in a form selected from the group consisting of base form, esters, hydrates, solvates, salts, and prodrugs.

In embodiments, the active pharmaceutical ingredient may be in a crystalline form having a block-like crystal habit or a needle-like crystal habit.

In embodiments, the active pharmaceutical ingredient may be formed by at least one milling technique selected from the group consisting of jet milling, nanomilling or wet milling in water or other aqueous solvent followed by lyophilization or drying, homogenization, ball milling, cutter milling, roller milling, grinding with mortar and pestle, runner milling, and cryomilling.

In embodiments, the biodegradable liquid polymer may comprise lactide and ε-caprolactone residues.

In embodiments, the ratio of lactide to ε-caprolactone residues may be from 60:40 to 90:10.

In embodiments, the ratio of lactide to ε-caprolactone residues may be 75:25.

In embodiments, the biodegradable liquid polymer may be formed with a hydroxy acid initiator.

In embodiments, the hydroxy acid initiator may be glycolic acid.

In embodiments, the biodegradable liquid polymer may have a weight average molecular weight between about 5 kDa and about 22 kDa.

In embodiments, the biodegradable liquid polymer may have a weight average molecular weight between about 5 kDa and about 16 kDa.

In embodiments, the biodegradable liquid polymer may have a weight average molecular weight between about 5 kDa and about 10 kDa.

In embodiments, the weight average molecular weight of the biodegradble liquid polymer may be between about 10 kDa and about 16 kDa.

In embodiments, the weight average molecular weight of the biodegradable liquid polymer may be between about 12 kDa and about 16 kDa.

In embodiments, the weight average molecular weight of the biodegradable liquid polymer may be between about 1 kDa and about 10 kDa, between about 1 kDa and about 8 kDa, or between about 1 kDa and about 5 kDa.

In embodiments, the composition may have a viscosity at room temperature suitable for injection through a needle having a gauge between about 16 and about 20.

In embodiments, the composition may have a shelf life of at least about three months at a temperature selected from room temperature and between 2° C. and 8° C.

In embodiments, the composition may form a liquid depot in a subject upon injection, wherein the liquid depot releases the API into the subject for a period of at least about 30 days.

In embodiments, the liquid depot may release the API into the subject for a period of at least about 60 days.

In embodiments, the liquid depot may release the API into the subject for a period of at least about 90 days.

In embodiments, the liquid depot may release the API into the subject for a period of at least about 120 days.

In embodiments, the composition may further include an additive, including, but not limited to, a surfactant. Examples of surfactants suitable for use in the invention include, but are not limited to, sucrose fatty acid esters.

It is another aspect of the present invention to provide a pharmaceutical composition having an active pharmaceutical ingredient in suspension, comprising an active pharmaceutical ingredient, consisting of testosterone or a pharmaceutically acceptable ester, hydrate, solvate, or prodrug thereof, or a salt of any of said ester, hydrate, solvate or prodrug; a biocompatible solvent system, consisting of approximately equal parts by weight N-methyl-2-pyrrolidone (NMP) and polyethylene glycol having a molecular weight of about 300 daltons (PEG 300); and an acid-initiated biodegradable liquid polymer having a weight-average molecular weight between about 1 kDa and about 25 kDa.

In embodiments, the biodegradable liquid polymer may be a D,L-lactide/ε-caprolactone copolymer, wherein a ratio of lactide monomer units to caprolactone monomer units in the copolymer is about 75:25.

In embodiments, the active pharmaceutical ingredient may make up about 20 wt % of the pharmaceutical composition, the biocompatible solvent system may make up up about 50 wt % of the pharmaceutical composition, and the biodegradable liquid polymer may make up about 30 wt % of the pharmaceutical composition.

In embodiments, the active pharmaceutical ingredient may have a D_{v,50} of between about 15 μm and about 200 μm and a particle size span of between about 1 and about 8.

It is another aspect of the present invention to provide a pharmaceutical composition, comprising about 20 wt % of testosterone undecanoate having a D_{v,50} particle size of between about 15 μm and about 200 μm; about 30 wt % of a glycolic acid-initiated liquid 75:25 D,L-lactide/ε-caprolactone copolymer having a weight-average molecular weight of between about 1 and about 25 kDa; between about 15 wt % and about 25 wt % of N-methyl-2-pyrollidone (NMP); and between about 25 wt % and about 35 wt % of polyethylene glycol having a molecular weight of about 300 daltons (PEG 300), where the wt % of each of the NMP and PEG 300 total 50 wt % in the composition.

In embodiments, the pharmaceutical composition may comprise about 15 wt % of the NMP and about 35 wt % of the PEG 300, wherein the D_{v,50} particle size of the testosterone undecanoate is between about 15 μm and about 20 μm, and the weight-average molecular weight of the copolymer is about 10-16 kDa.

In embodiments, the pharmaceutical composition may comprise about 25 wt % of the NMP and about 25 wt % of the PEG 300, wherein the D_{v,50} particle size of the testosterone undecanoate is between about 15 μm and about 90 μm, and the weight-average molecular weight of the copolymer is about 10-16 kDa.

In embodiments, the pharmaceutical composition may comprise about 25 wt % of the NMP and about 25 wt % of the PEG 300, wherein the D_{v,50} particle size of the testosterone undecanoate is between about about 35 μm and about 90 μm, and the weight-average molecular weight of the copolymer is about 10-16 kDa.

In embodiments, the weight-average molecular weight of the copolymer is between about 14 and 16 kDa.

In embodiments, the weight-average molecular weight of the copolymer is between about 1 and 5 kDa, between about 1 and about 10 kDa, or between about 1 and about 12 kDa.

It is another aspect of the present invention to provide a pharmaceutical composition, comprising about 15 wt % of testosterone undecanoate; about 20 wt % of a glycolic acid-initiated liquid 75:25 D,L-lactide/ε-caprolactone copolymer having a weight-average molecular weight of between about 1 kDa and about 25 kDa; and about 65 wt % benzyl benzoate.

In embodiments, the weight-average molecular weight of the copolymer may be between about 1 kDa and about 5 kDa, about 5 kDa, about 8.5 kDa, about 10 kDa, about 12 kDa, about 14 kDa, about 14.2 kDa, about 15 kDa, about 15.5 kDa, or about 22 kDa.

In embodiments, the active pharmaceutical ingredient may have a particle size span of between about 1 and about 8.

It is another aspect of the present invention to provide a pharmaceutical composition for use as a medicament or in the treatment of a disease, comprising an active pharmaceutical ingredient having a log P of at least about 0; a biocompatible solvent or combination or mixture of solvents and/or co-solvents; and a biodegradable liquid polymer, wherein at least one of the following is true: the biodegradable liquid polymer has a weight average molecular weight of between about 1 kDa and about 25 kDa; the active pharmaceutical ingredient is in substantially solid form in the biocompatible solvent or combination or mixture of solvents and/or co-solvents at body temperature; and the active pharmaceutical ingredient has a D_{v,50} of between about 1 μm and about 250 μm and a particle size span of between about 1 and about 8.

It is another aspect of the present invention to provide a method of testosterone supplementation in a subject, comprising administering to the subject a pharmaceutical composition of the invention, wherein the active pharmaceutical ingredient comprises at least one of testosterone, an ester, complex, hydrate, solvate, or prodrug of testosterone, and a salt of any of said esters, complexes, hydrates, solvates, and prodrugs.

In embodiments, a serum testosterone level of the subject may be between about 3 ng/mL and about 10 ng/mL for at least about one month after the administering step.

In embodiments, a serum testosterone level of the subject may be between about 3 ng/mL and about 10 ng/mL for at least about two months after the administering step.

In embodiments, a serum testosterone level of the subject may be between about 3 ng/mL and about 10 ng/mL for at least about three months after the administering step.

It is another aspect of the present invention to provide a method of treating a subject, comprising administering to the subject a pharmaceutical composition of the invention.

In embodiments, the active pharmaceutical ingredient of the pharmaceutical composition may be testosterone undecanoate.

In embodiments, the active pharmaceutical ingredient of the pharmaceutical composition may be testosterone cypionate.

In embodiments, a pharmaceutical composition of the invention is administered subcutaneously to a subject.

It is another aspect of the present invention to provide a malleable, non-rigid, non-solid implant formed upon administration of a pharmaceutical composition of the invention.

### BRIEF DESCRIPTION OF THE DRAWINGS

FIGS. 1A and 1B show the testosterone undecanoate release rate (mg/day), and the percentage testosterone undecanoate (TU) released over time, respectively, for four Liquid Polymer Technology (LPT)-TU formulations of the invention in an in vitro release assay (LPT-TU Test Formulations 1 (▪), 2 (▴) and 3 (∘), and Control LPT Formulation (X).

FIG. 1C shows the temperature at which the suspended drug in the three LPT-TU formulations from Table 3 becomes fully dissolved and the formulation forms a solution.

FIG. 2 shows the results of an in vivo experiment comparing the mean testosterone concentration (ng/mL) in rats after injection with various LPT-TU Test Formulations of the invention (LPT-TU Test Formulations 1 (ζ), 2 (▴) and 3 (∘), and Non-Polymeric TU Control Solution (□)).

FIGS. 3A and 3B show the in vitro TU release rate (mg/day) and percentage testosterone undecanoate (TU) released over time, respectively, for four LPT-TU formulations comprising polymer having a weight average molecular weight of approximately 10 kDa, where the TU particle size and amount of co-solvent in the formulation were varied (LPT-TU Test Formulations 4 (▪); Test Formulation 5 (▴); Test Formulation 6 (●); Test Formulation 7 (♦)).

FIGS. 3C and 3D show the in vitro TU release rate (mg/day) and percentage testosterone undecanoate (TU) released over time, respectively, for four LPT-TU formulations comprising polymer having a weight average molecular weight of approximately 14 kDa, where the TU particle size and amount of co-solvent in the formulation were varied (LPT-TU Test Formulations 8 (▪); Test Formulation 9 (∘); Test Formulation 10 (⋄); Test Formulation 11 (Δ)).

FIGS. 3E and 3F show the in vitro TU release rate (mg/day) and percentage testosterone undecanoate (TU) released over time, respectively, for four LPT-TU formulations comprising polymer having a weight average molecular weight of approximately 22 kDa, where the TU particle size and amount of co-solvent in the formulation were varied (LPT-TU Test Formulations 12 (--X--); Test Formulation 13 (--+--); Test Formulation 14 ( [symbol] ); Test Formulation 15 (--□--)).

FIGS. 4A and 4B show the in vitro TU release rate (mg/day) and percentage testosterone undecanoate (TU) released over time, respectively, for four LPT-TU formulations comprising TU having a D_{v,50} particle size of approximately 15 μm, where the weight average molecular weight of the polymer and the amount of co-solvent in the formulation were varied (LPT-TU Test Formulations 4 (▪); Test Formulation 7 (♦); Test Formulation 8 (□); Test Formulation 12 (--X--); and Test Formulation 14 ( [symbol] ).

FIGS. 4C and 4D show the in vitro TU release rate (mg/day) and percentage testosterone undecanoate (TU) released over time, respectively, for four LPT-TU formulations comprising TU having a D_{v,50} particle size of approximately 56 μm, where the weight average molecular weight of the polymer and the amount of co-solvent in the formulation were varied (LPT-TU Test Formulations 6 (●); Test Formulation 9 (∘); and Test Formulation 10 (⋄)).

FIGS. 4E and 4F show the in vitro TU release rate (mg/day) and percentage testosterone undecanoate (TU) released over time, respectively, for four LPT-TU formulations comprising TU having a D_{v,50} particle size of approximately 64 μm or 90 μm, where the weight average molecular weight of the polymer and the amount of co-solvent in the formulation were varied (LPT-TU Test Formulations 5 (▴); Test Formulation 11 (Δ); Test Formulation 13 (--+--); and Test Formulation 15 (--□--)).

FIG. 4G shows the temperature at which the suspended drug in eleven of the LPT-TU formulations from Table 4 becomes fully dissolved and the formulation forms a solution.

FIGS. 5A and 5B show the in vitro TU release rate (mg/day) and percentage testosterone undecanoate (TU) released over time, respectively, for five LPT-TU formulations comprising the same polymer and solvent system, but where the D_{v,50} particle size of the TU in the formulation was varied (Test Formulation 16 (6 μm TU; □), Test Formulation 17 (15 μm TU; ⋄), Test Formulation 18 (56 μm TU; ∘), Test Formulation 19 (64 μm TU; X), and Test Formulation 20 (86 μm TU; ♦).

FIGS. 5C and 5D show the in vitro TU release rate (mg/day) and percentage testosterone undecanoate (TU) released over time, respectively, for five LPT-TU formulations comprising the same polymer and solvent system, but where the particle size distribution of the TU in the formulation was varied (6 μm TU (100%), ●); 64 μm/6 μm (60%/40%), Δ; 64 μm/6 μm (80%/20%), ⋄; and 64 μm (100%), ▪).

FIGS. 6A and 6B show the in vitro TU release rate (mg/day) and percentage testosterone undecanoate (TU) released over time, respectively, for four different LPT-TU formulations of the invention (Test Formulation 2 (●); Test Formulation 21 (□), Test Formulation 22 (▴) and Test Formulation 23 (⋄)).

FIG. 7 shows the results of an in vivo experiment comparing the mean testosterone concentration (ng/mL) in rats after injection with various LPT-TU Test Formulations of the invention (Test Formulation 21(●), Test Formulation 22 (X), and Test Formulation 23 (Δ); Non-Polymeric TU Control Solution (□).

FIG. 8 shows the results of an in vivo experiment comparing the mean testosterone concentration (ng/mL) in minipigs after injection with various LPT-TU Test Formulations of the invention (Test Formulation 22 (X) and Test Formulation 23 (Δ)).

FIG. 9 shows the results of an in vivo experiment comparing the mean testosterone concentration (ng/mL) in minipigs after injection with various LPT-TU Test Formulations of the invention (Non-Polymeric TU Control Solution Group A one dose (□); Non-Polymeric TU Control Solution Group A two doses (X); Test Formulation Group C (★); Test Formulation Group D (⋆); Test Formulation Group E (⋄); Test Formulation Group F( [symbol] )).

FIGS. 10A and 10B show the in vitro TU release rate (mg/day) and percentage testosterone undecanoate (TU) released over time, respectively, for five LPT-TU formulations in which the TU is in solution in the formulation, as compared to an LPT-TU suspension control (Test Formulation A (□); Test Formulation B (∘); Test Formulation C (Δ); Test Formulation D (♦); Test Formulation E (▪) and Control Suspension (●)).

FIG. 11 shows the results of an in vivo experiment comparing the mean testosterone concentration (ng/mL) in rats after injection with an LPT-TU Solution Test Formulation of the invention (Test Formulation A ( [symbol] ), Non-Polymeric TU Control Solution (□)).

FIG. 12 shows the results of an in vivo experiment comparing the mean testosterone concentration (ng/mL) in rats after injection with various LPT-TU Solution and Suspension Test Formulations of the invention (Test Formulation C (♦); Test Formulation D (⋆); Test Formulation 2 (▴); and Non-Polymeric TU Control Solution (□)).

FIG. 13 shows the temperature at which the suspended drug in six of the LPT-TC formulations of the invention becomes fully dissolved and the formulation forms a solution.

FIGS. 14A and 14B show the testosterone cypionate (TC) release rate (mg/day), and the percentage TC released over time, respectively, for three LPT-TC formulations of the invention in an in vitro release assay (LPT-TC Test Formulations 1 (♦), 2 (▴), and 4 (▪)).

### DETAILED DESCRIPTION OF THE INVENTION

Certain active pharmaceutical ingredients (APIs) have relatively low solubility in aqueous media and/or relatively high hydrophobicity (i.e., relatively low hydrophilicity). Such APIs may be more difficult to solubilize in pharmaceutically acceptable solvent systems and/or may be more likely to remain in solid form, i.e. in suspension, in such solvent systems as compared to APIs having higher aqueous solubility and/or lower hydrophobicity (i.e., higher hydrophilicity). Such APIs are also less likely to dissolve in biological fluids (e.g., plasma, gastric juices) and may thus have lower bioavailability, especially when provided in certain dosage forms, including oral dosage forms and parenteral dosage forms, and it can be especially difficult to formulate compositions of these APIs that allow for extended release of the drug.

The present invention provides pharmaceutical compositions of hydrophobic and/or poorly water-soluble APIs that are suitable for, among other uses, extended release of the API upon administration to a patient. The present invention achieves this and other benefits by the creation of stable suspensions of the API, or alternatively stable solutions of the API, in an extended release form, which the present invention accomplishes by the inclusion of a liquid polymer/solvent system in the pharmaceutical formulation, to form a “liquid polymer composition” or “liquid polymer formulation” (also referred to herein as a Liquid Polymer Technology (LPT) composition or formulation). When pharmaceutical compositions (formulations) are provided using the liquid polymer/solvent system of the invention and using the guidance provided herein, the API that is in solid form (suspension) in such a formulation advantageously remains in a stable physical form (e.g., the API does not undergo a phase change, or remains in substantially solid or suspension form) at room temperature (i.e., during manufacture, transportation, and/or storage) and at body temperature (i.e., in vivo within the body of the patient and when exposed to the internal environment of the body of the patient). In some embodiments of the invention, the API which is in suspension in the formulation also remains in a stable physical form within the inventive formulations at temperatures higher than body temperature, such as temperatures that may be experienced during terminal sterilization processes, including electron beam (e-beam) irradiation. In some embodiments of the invention, the API is in a solution form in the formulation (i.e., is substantially or fully dissolved in the formulation), as described in more detail below. The formulations of the invention in which the API is in solution form are also stable (i.e., the API does not undergo phase changes, such as by precipitating in the formulation) over a large range of temperatures. In other words, liquid polymer compositions of the invention that provide the API in suspension are stable in that the API does not readily dissolve in the formulation, and liquid polymer compositions of the invention that provide the API in solution are stable in that the API does not readily precipitate in the formulation.

Pharmaceutical liquid polymer compositions of the invention are also characterized in that they remain substantially stable at cold storage temperatures (e.g., refrigeration temperatures), meaning that the compositions do not freeze and/or do not show an unacceptable degree of degradation when stored at these temperatures over a reasonable extended period of time.

Moreover, the liquid polymer compositions of the invention remain in liquid form in vivo, i.e., liquid polymer compositions of the invention do not form a solid implant in vivo, even after the solvent has dissipated from the polymer upon exposure to the aqueous environment in the body.

Without wishing to be bound by any particular theory, it is believed that the liquid polymer pharmaceutical compositions of the present invention are stable at temperatures ranging from cold storage temperatures (or lower), up to room temperature, or up to in vivo temperatures, or even up to higher temperatures as a result of a combination of factors. Such factors include at least the chosen solvent or solvent system and the molecular weight and composition of the liquid polymer. Liquid polymer pharmaceutical compositions of the present invention are also suitable for use as extended release formulations as a result of a combination of factors, including at least the chosen solvent or solvent system and the molecular weight and composition of the liquid polymer, and the particle size of the API (when the formulation provides the API in suspension). One or more of these factors may affect one or more of the other factors; by way of non-limiting example, certain solvents or solvent systems affect the stability of the liquid polymer, particularly at a given temperature (e.g., refrigeration temperatures), and thus affect the stability of the pharmaceutical composition as a whole. Similarly, certain solvents or solvent systems affect the stability of the physical form of the API when provided within the liquid polymer formulation throughout a large range of temperatures, including temperatures experienced in an in vivo environment.

Accordingly, the present invention is directed to biodegradable liquid polymer technology (LPT) pharmaceutical compositions that can be administered into the body with syringes or needles and that are utilized to deliver a drug (active pharmaceutical ingredient, or API) into the body over an extended period of time. Such compositions can deliver APIs to a patient at consistent levels within a therapeutic window for long periods of time to allow for improved ease of administration, resulting in improved patient compliance with administration protocols. In particular, the present invention is directed to LPT pharmaceutical compositions, also referred to as LPT formulations, which include a biodegradable polymer, a solvent or combination or mixture of solvents and/or co-solvents, and an active pharmaceutical ingredient (API) that is characterized as having relatively low solubility in aqueous media and/or relatively high hydrophobicity (i.e. relatively low hydrophilicity). The LPT formulations of the invention remain liquid after administration to the body (e.g., the formulations do not form solid implants, as discussed in detail herein), and LPT formulations remain stable with respect to both the polymer and the API over a wide range of temperatures.

By way of illustrating the present invention, during development of an LPT formulation for the delivery of an API having relatively low solubility in aqueous solutions, namely testosterone undecanoate (TU), an LPT-TU formulation was designed to incorporate the API in substantially solid form (i.e., as a suspension). The LPT-TU formulation was comprised of 20 wt % TU, 30 wt % LPT polymer (75:25 DL-lactide/ε-caprolactone liquid copolymer), and 50 wt % N-methyl-2-pyrrolidone (NMP). This formulation provided an injectable extended release formulation for the delivery of testosterone prodrug (in this case, TU) in the form of an oil-free formulation, which eliminated or reduced the risk of pulmonary oil microembolism (a risk associated with one of the current commercial injectable products for the delivery of TU). However, it was unexpectedly discovered that when the formulation was terminally sterilized using e-beam irradiation, which is one method used to terminally sterilize a product prior to injection, sample temperatures increased to −35-40° C., and the suspended TU dissolved into the polymer matrix. As the samples cooled, TU crystallized in an uncontrolled fashion. This led to unacceptable variability in the TU particle size, which affected injectability of the formulation.

Therefore, a TU recrystallization method was developed, and implemented after e-beam irradiation. This allowed for control of TU particle size within the formulation, which in turn provided for acceptable injectability with the target needle gauge. However, the recrystallized LPT-TU formulation surprisingly had a decreased degradation rate, altered degradation mode, exhibited less desirable in vitro release kinetics, and exhibited slow in vivo release that did not maintain testosterone levels within the targeted therapeutic range.

To address these issues, the inventors designed and developed new liquid polymer formulations suitable for use with TU (which are expected also be useful for APIs similar to TU, such as APIs that have relatively low solubility in aqueous environments and highly variable solubility in organic solvents, dependent on the characteristics of the solvent). These formulations were designed to be suitable for use in a clinical product and with the desired manufacturing processes for such products, including desired sterilization processes. The new formulations were designed to have most or all of the following characteristics, using TU as an API that is exemplary of the present invention:
Comprised of a biocompatible solvent (or solvent and co-solvent), biodegradable liquid polymer, and API, with other additives being acceptable if necessary and safe for injection (e.g., by parenteral injection, including, but not limited to subcutaneous or intramuscular injection);
Exists as either a solution, or a suspension with controlled particle size;
Has a viscosity sufficiently low to facilitate resuspension (if necessary) and injection;
API within the formulation should not undergo phase transitions within the temperature range of refrigeration temperatures (about 2-8° C.) up to at least body temperatures (about 36.5° C. to about 37.5° C.), and/or up to at least 40-45° C. or higher;
Compatible with terminal sterilization processes (e.g., electron beam (e-beam) irradiation, gamma irradiation, X-ray irradiation);
Can be delivered via subcutaneous injection using a small gauge needle (>20 G);
Forms a non-solid, soft implant which ideally does not impact physical mobility when injected into the body;
Meets stability requirements as either a room temperature or refrigerated product for ≥2 years;
Specifically for TU and related drugs (e.g., testosterone cypionate), provides testosterone supplementation in the eugonadal range (10.4-34.7 nmol/L or 3-10 ng/mL testosterone in plasma (see, e.g., Shehzad Basaria, “Male hypogonadism,” 383 Lancet 1250 (2014) (hereinafter “Basaria”); Abraham Morgentaler et al., “Long acting testosterone undecanoate therapy in men with hypogonadism: results of a pharmacokinetic clinical study,” 180 J. Urology 2307 (2008) (hereinafter “Morgentaler”)):
   ≥75% patients have total testosterone C_{avg} from 3-10 ng/mL;
   The lower limit of the 95% CI for percent of subjects with C_{avg} within the eugonadal range is >65%;
Specifically for TU and related drugs (e.g., testosterone cypionate), has acceptable Cₘₐₓ, per USFDA thresholds (e.g., see Morgentaler et al., supra);
   No instances of Cₘₐₓ≥25 ng/mL;
   σ<5% Cₘₐₓ between 18 and 25 ng/mL;
   ≥85% Cₘₐₓ≤15 ng/mL; and
Specifically for TU and related drugs (e.g., testosterone cypionate), provides testosterone supplementation for at least 8, 9 or 10 weeks, and in some embodiments, at least 11 or 12 weeks, and in some embodiments, greater than 12 weeks.

Multiple new LPT formulations were designed utilizing a variety of: LPT copolymers and polymer ratios, polymer molecular weights, solvents and solvent combinations, additives, drug processing steps (for suspensions), and drug/polymer/solvent ratios. In particular, the inventors designed LPT formulations with the goals of: (1) increasing drug release and depot degradation in vivo, while maintaining the extended release capability of the formulations; (2) forming LPT solution formulations that were stable within target temperature ranges and time periods; and (3) forming LPT suspension formulations that were stable within target temperature ranges and time periods. These LPT formulations were then evaluated for characteristics including: viscosity/injectability, drug (e.g., TU or TC) solubility in the formulation, liquid depot degradation rate, polymer stability, in vitro drug (e.g., TU or TC) release, formulation freeze temperatures (to evaluate phase stability at refrigeration temperatures), and drug (e.g., TU or TC) dissolution temperatures, e.g., temperatures where drug in suspension in the formulation dissolves and the formulation becomes a solution (to evaluate phase stability at higher temperatures, such as those experienced during e-beam irradiation).

### Definitions

As used herein, the term “animal” refers to any organism of the kingdom Animalia. Examples of “animals” as that term is used herein include, but are not limited to, humans (Homo sapiens); companion animals, such as dogs, cats, and horses; and livestock animals, such as cows, goats, sheep, and pigs.

As used herein, the term “biocompatible” means “not harmful to living tissue.”

As used herein, the term “biodegradable” refers to any water-insoluble material that is converted under physiological conditions into one or more water-soluble materials, without regard to any specific degradation mechanism or process.

As used herein, the term “co-solvent” refers to a substance added to a solvent to increase or modify the solubility of a solute in the solvent.

As used herein, the term “liquid” refers to the ability of a composition to undergo deformation under a shearing stress, regardless of the presence or absence of a non-aqueous solvent. Liquid polymer compositions and the liquid polymers (also referred to as “liquid polymers) according to the invention have a liquid physical state at ambient and body temperatures and remain liquid in vivo, i.e., in a largely aqueous environment. The liquid polymer compositions and liquid polymers have a definite volume, but are an amorphous, non-crystalline mass with no definite shape. In addition, the liquid polymers according to the invention are not soluble in body fluid or water and therefore, after injection into the body and dissipation of the solvent, remain as a cohesive mass when injected into the body without themselves significantly dissipating. In addition, such liquid polymer compositions can have a viscosity, density, and flowability to allow delivery of the composition through standard gauge or small gauge needles (e.g., 18-26 gauge) with low to moderate injection force using standard syringes. The liquid polymers of the present invention are further characterized as not forming a solid implant in situ in the body when injected into the body as part of a sustained release drug delivery system that includes the liquid polymers and a biocompatible solvent. In other words, liquid polymers according to the present invention remain in a substantially liquid form in situ upon exposure to an aqueous environment, such as upon injection into the body, including after the solvent in the administered composition has dissipated. The liquid polymers of the present invention can be further characterized being non-crystalline, amorphous, non-thermoplastic, non-thermosetting, and/or non-solid. “Liquids,” as that term is used herein, may also exhibit viscoelastic behavior, i.e. both viscous and elastic characteristics when undergoing deformation, such as time-dependent and/or hysteretic strain. By way of non-limiting example, viscoelastic materials that are generally flowable but have a partially solid character and/or a plastic- or gel-like character, such as cake batter or raw pizza dough, and similar materials, are “liquids” as that term is used herein. In some embodiments, materials having a non-zero yield stress that do not deform at stresses below the yield stress, and that are readily deformable without a characteristic of material fracture or rupture at materials above the yield stress, may be “liquids” as that term is used herein.

As used herein, the terms “molecular weight” and “average molecular weight,” unless otherwise specified, mean a weight-average molecular weight as measured by a conventional gel permeation chromatography (GPC) instrument (such as an Agilent 1260 Infinity Quaternary LC with Agilent G1362A Refractive Index Detector) utilizing polystyrene standards and tetrahydrofuran (THF) as the solvent.

As used herein, the terms “patient” and “subject” are interchangeable and refer generally to an animal to which a composition or formulation of the invention is administered or is to be administered.

As used herein, the term “polymer” refers generally to polymers, copolymers and/or terpolymers formed of repeating units, which can be linear, branched, grafted and/or star-shaped. Non-limiting examples of polymers include polyglycolides, polylactides, polycaprolactones, polyanhydrides, polyorthoesters, polydioxanones, polyacetals, polyesteramides, polyamides, polyurethanes, polycarbonates, polyphosphazenes, polyketals, polyhydroxybutyrates, polyhydroxyvalerates, polyethylene glycols, polyesters, and polyalkylene oxalates. Water-insoluble polymers that are converted under physiological conditions into one or more water-soluble materials are referred to as herein as “biodegradable polymers,” and non-limiting examples of biodegradable polymers include co-polymers or terpolymers comprising: lactide monomers and caprolactone monomers, lactide monomers and trimethylene carbonate monomers, or lactide monomers and dioxanone monomers.

As used herein, the term “small molecule” means an organic compound having a molecular weight less than 900 daltons.

As used herein, the term “solvent” refers to a liquid that dissolves a solid or liquid solute, or to a liquid external phase of a suspension throughout which solid particles are dispersed.

As used herein, the term “solubilizer” refers to a compound that increases the solubility of another substance. Examples of solubilizers useful in the present invention include any solubilizer useful for parenteral injection, and include, but are not limited to, surfactants and other solubilizers, such as Poloxamer 188, sorbitan trioleate, lecithin (e.g., soya or egg), Vitamin E TPGS, sugar based esters or ethers (e.g., sugar acid esters of fatty alcohols or sugar alcohol esters of fatty acids, including, but not limited to, sucrose cocoate, sucrose stearate, sucrose laurate, etc,), amino acid-based solubility enhancers (e.g., proline, arginine, DL-methionine), and protein-based solubility enhancers (e.g., hydrophobins).

As used herein, the term “surfactant” refers to a compound that lowers the surface tension between two liquids, between a gas and a liquid, or between a liquid and a solid. For example, a surfactant can act as a wetting agent, which aids in dispersing an active pharmaceutical ingredient in a liquid vehicle, or as a solubilizer.

As used herein, the term “sucrose fatty acid ester” or “sucrose ester” or “sugar ester” or “sucrose fatty acid ether” or “sucrose ether” or “sugar ether” refers to a group of surfactants chemically synthesized from esterification or etherification, respectively, of a sugar, sugar alcohol, or sugar derivative (e.g, sucrose or other sugar) and fatty acids (or glycerides) or fatty alcohols. Because they have amphiphilic properties, they have the ability to bind to both water and oil simultaneously and are thus useful as emulsifiers or stabilizers.

Unless otherwise specified, all ratios between monomers in a copolymer disclosed herein are molar ratios.

Unless otherwise specified, all particle sizes and particle size distributions disclosed herein are determined according to volume-based particle size measurements, such as, by way of non-limiting example, by use of a laser diffraction particle size analyzer such as a Malvern Mastersizer® instrument. Software programs and calculations that can convert from a number-based distribution analysis to a volume-based distribution analysis (and vice versa) are well known in the art; therefore, for particle sizes calculated using a number-based method, a volume-based particle size can also be estimated. Volume-based particle size distribution measurements are the default choice for many ensemble light scattering particle size measurement techniques, including laser diffraction, and are generally used in the pharmaceutical industry.

One embodiment of the invention is a pharmaceutical composition having an active pharmaceutical ingredient (API) in suspension where the API is characterized as having relatively low solubility in aqueous media and/or relatively high hydrophobicity (i.e. relatively low hydrophilicity). The formulation includes such an API (e.g., an API having an octanol-water partition coefficient of at least about 1), a biocompatible solvent or combination or mixture of solvents and/or co-solvents, and a biodegradable liquid polymer having a molecular weight between about 1 kDa and about 25 kDa. The API is substantially in solid form (in suspension) in the liquid polymer and solvent(s) and the API does not undergo phase transition (e.g., does not substantially dissolve in the formulation, remains in suspension, or remains in substantially solid form) in the liquid polymer and solvent(s) at temperatures up to at least body temperature (e.g., about 36.5° C. to about 37.5° C. (about 97.7° F. to about 99.5° F.)). In one embodiment, the API is in substantially solid form in the liquid polymer and solvent(s) up to temperatures that are higher than body temperatures, such as temperatures up to 40-45° C. In one embodiment, the API remains substantially in solid form (the API does not undergo a phase transition) in the liquid polymer and solvent(s) up to at least 45° C. or higher. In one embodiment, the liquid polymer composition (i.e., the composition including the liquid polymer, solvent(s) and API) does not undergo a phase transition (e.g., does not freeze) at refrigeration temperatures e.g., between about 2° C. and 8° C. In one embodiment, the active pharmaceutical ingredient has a volume-based particle size distribution median (D_{v,50}) of between about 15 μm and about 200 μm and a particle size span of between about 1 and about 8. In one embodiment, and by way of example, the API has an octanol-water partition coefficient of at least about 1. In one embodiment, such an API has a log P of greater than 0. In one embodiment, such an API has a log P of greater than about 5.

Another embodiment of the invention is a pharmaceutical composition having an active pharmaceutical ingredient (API) in solution where the API is characterized as having relatively low solubility in aqueous media and/or relatively high hydrophobicity (i.e. relatively low hydrophilicity). The formulation includes such an API (e.g., an API having an octanol-water partition coefficient of at least about 1), a biodegradable liquid polymer having a molecular weight between about 1 kDa and about 25 kDa, and a biocompatible solvent or combination or mixture of solvents and/or co-solvents, where the API is substantially or fully dissolved (in solution) in the polymer/solvent formulation. In this embodiment, the API does not undergo phase transition (e.g., does not come out of solution) in the composition when exposed to a variety of temperatures, e.g., temperatures ranging from about 2° C. or lower to at about 38° C. or higher.

### Active Pharmaceutical Ingredient (API)

APIs suitable for use in embodiments of the present invention generally include drugs having low solubility in aqueous media and/or relatively high hydrophobicity (i.e., relatively low hydrophilicity), and which thus are more difficult to solubilize in pharmaceutically acceptable solvent systems and/or may be more likely to remain in solid form, i.e., in suspension, in such polymer/solvent systems as compared to APIs having higher aqueous solubility and/or lower hydrophobicity (i.e. higher hydrophilicity). Hydrophobic and/or poorly water-soluble APIs that are intended to be released and/or administered to a patient over a period of multiple weeks or months may be especially desirable for use in the present invention, given the difficulty of formulating extended release compositions of these APIs by the methods and systems of the prior art.

Low solubility in aqueous media can be determined by different methods known to those in the art and in some embodiments, will include APIs having an octanol-water partition coefficient (P) of at least about 1, i.e. a log(P) of at least about 0, and such APIs may often have a P of at least about 100,000, i.e. a log(P) of at least about 5. Thus, the log(P) of APIs used in the present invention may be at least about 0, at least about 1, at least about 2, at least about 3, at least about 4, at least about 5, at least about 6, at least about 7, or at least about 8, or in other embodiments at least about any tenth of an integer between 0 and 8, i.e. at least about 0, at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8.

A partition coefficient (e.g., octanol-water partition coefficient, using e.g., 1-octanol) is a measure of the relative hydrophobicity and hydrophilicity of a compound, and more particularly, a partition coefficient describes the propensity of a neutral (uncharged) compound to dissolve in an immiscible biphasic system of lipid (fats, oils, organic solvents) and water. In simple terms, it measures how much of a solute dissolves in the water portion versus an organic portion. When log(P) is zero, the compound is equally partitioned (equally soluble) between lipid and aqueous phases; when the log(P) is greater than 0, the compound is more lipophilic (or hydrophobic), meaning that the compound is more soluble in a lipid phase, and when the log(P) is less than 0, the compound is more hydrophilic, meaning that the compound is more soluble in an aqueous phase.

APIs suitable for use in embodiments of the present invention generally include any drug that (1) is suitable or intended for extended release in a body of a patient for a period of at least about one week up to a period of at least about six months, and (2) does not chemically interact with the acid end groups of the biodegradable liquid polymer. Where the API is ionizable, a pKa of the API is typically greater than about 3 and less than about 8.5.

A further consideration in the design of the formulations of the present invention is the chemical and physical stability of the API in the formulation as a function of temperature. For example, although formulations according to the present invention may be administered at approximately room temperature and may be subjected to human body temperature for a period of up to about six months, the formulations of the present invention may often be subjected to electron-beam (“e-beam”) irradiation to sterilize the formulations for use in humans. The temperature of the formulation during e-beam processing may reach as high as 20° C., or as high as near body temperature (e.g., 34° C., 35° C., 36° C., 37° C., 38° C. or higher), and in some circumstances could reach as high as 45° C., unless the temperature is controlled by using refrigerated processing or by using split dose processing methods. It is important for the API of choice to be chemically and physically stable within the formulation at ambient temperature and at body temperatures, and in one embodiment, also at the higher temperatures associated with, for example, certain e-beam irradiation processes or other processes which may expose the liquid polymer formulation to an elevated temperature for a period of time. Similarly, because formulations of the present invention may be stored for weeks or months at ambient temperatures or under refrigeration, chemical and physical stability of the API within the formulations in this lower temperature range is also an element of the invention. As disclosed more fully throughout this Detailed Description, formulations comprising drugs meeting chemical and physical stability requirements across the full range of applicable temperatures include, but are by no means limited to, liquid polymer formulations comprising testosterone, hormones and steroids other than testosterone, APIs having similar low solubility in aqueous environments as testosterone undecanoate, and pharmaceutically acceptable salts and esters of any of such APIs.

In one embodiment, the API is in substantially solid form in the liquid polymer and solvent(s) composition at temperatures up to body temperature (e.g., about 36.5° C. to about 37.5° C. (about 97.7° F. to about 99.5° F.)). In one embodiment, the API is in substantially solid form in the liquid polymer and solvent(s) composition at temperatures up to at least about 36° C. In one embodiment, the API is in substantially solid form in the liquid polymer and solvent(s) composition at temperatures up to at least about 37° C. In one embodiment, the API is in substantially solid form in the liquid polymer and solvent(s) composition at temperatures up to at least about 38° C. In one embodiment, the API is in substantially solid form in the liquid polymer and solvent(s) composition at temperatures up to at least about 39° C. In one embodiment, the API is in substantially solid form in the liquid polymer and solvent(s) composition at temperatures up to at least about 40° C. In one embodiment, the API is in substantially solid form in the liquid polymer and solvent(s) composition at temperatures up to at least about 41° C. In one embodiment, the API is in substantially solid form in the liquid polymer and solvent(s) composition at temperatures up to at least about 42° C. In one embodiment, the API is in substantially solid form in the liquid polymer and solvent(s) composition at temperatures up to at least about 43° C. In one embodiment, the API is in substantially solid form in the liquid polymer and solvent(s) composition at temperatures up to at least about 44° C. In one embodiment, the API is in substantially solid form in the liquid polymer and solvent(s) composition at temperatures up to at least about 45° C. In one embodiment, the API is in substantially solid form in the liquid polymer and solvent(s) composition at a temperature range spanning from refrigeration temperature (e.g., 2-8° C.) or lower up to body temperature, or in other embodiments up to any temperature between 36° C. and 45° C. or higher, in 0.1° C. increments. In one embodiment, the temperature at which the API becomes fully dissolved in the polymer and solvent or combination or mixture of solvents, and the formulation thus becomes a solution, is 45° C. or higher (e.g., 46° C., 47° C., 48° C., 49° C., 50° C., 51° C., 52° C., 53° C., 54° C., 55° C., or higher than 55° C.).

In one embodiment, the API in a liquid polymer formulation of the invention is in solution in the formulation. In one embodiment, the API in an liquid polymer formulation of the invention is in solution in the formulation at a temperature range spanning from refrigeration temperature (e.g., 2-8° C.) or lower up to body temperature, or in other embodiments up to any temperature between 36° C. and 45° C. or higher, in 0.1° C. increments.

APIs (also referred to herein as drugs or active pharmaceutical agents) that are suitable for the present application are biologically active agents that provide a biological effect and that act locally or systemically in the treatment, therapy, cure and/or prevention of a disease, disorder, or other ailment, or otherwise provide a health or medical benefit to a subject. Examples of such drugs include, without limitation, antimicrobials, anti-infectives, anti-parasitic drugs such as avermectins, anti-allergenics, steroidal anti-inflammatory agents, non-steroidal anti-inflammatory agents, anti-tumor agents, anticancer drugs, decongestants, miotics, anti-cholinergics, sympathomimetics, sedatives, hypnotics, psychic energizers, tranquilizers, endocrine/metabolic agents, hormones (e.g. androgen, anti-estrogen, estrogen, gonadotropin-releasing hormone analogues, testosterone and progesterone), drugs for the treatment of diabetes, drugs for the treatment of dementia (e.g. Alzheimer's disease), GLP-1 agonists, androgenic steroids, estrogens, progestational agents, LHRH agonists and antagonists, somatotropins, narcotic antagonists, prostaglandins, analgesics, antispasmodics, antimalarials, antihistamines, cardioactive agents, antiparkinsonian agents, antihypertensive agents, anti-virals, antipsychotics, immunosuppressants, anesthetics, antifungals, antiproliferatives, anticoagulants, antipyretics, antispasmodics, and nutritional agents. APIs of the foregoing classes and specific APIs described herein can be administered in various forms, including as base form, salts, esters, complexes, prodrugs and analogs of the foregoing.

API's useful in the invention include a small molecule organic compound. The small molecule drug may be a hydrophobic drug, such as corticosteroids such as prednisone, prednisolone, beclomethasone, fluticasone, methylprednisone, triamcinolone, clobetasol, halobetasol, and dexamethasone; azole medications such as metronidazole, fluconazole, ketoconazole, itraconazole, miconazole, dimetridazole, secnidazole, ornidazole, tinidazole, carnidazole, and panidazole; sex steroids such as testosterone, estrogens such as estradiol, and progestins, including esters thereof; statin drugs such as atorvastatin, simvastatin, fluvastatin, lovastatin, pitavastatin, pravastatin, and rosuvastatin; and antiandrogen drugs such as abiraterone, galeterone, orteronel, and enzalutamide and salts, esters, complexes, prodrugs and analogs of the foregoing.

Examples of specific additional drugs that may be utilized include hydrophilic and hydrophobic small molecule drugs such as rivastigmine tartrate, cisplatin, carboplatin, paclitaxel, rapamycin, tacrolimus (fujimycin), bortezomib, trametinib, methotrexate, riociguat, macitentan, sumatriptan, anastozole, fulvestrant, exemestane, misoprostol, follicle-stimulating hormone, axitinib, paricalcitol, pomalidomide, dustasteride, doxycycline, doxorubicin, ciprofloxacin, quinolone, ivermectin, eprinomectin, doramectin, leflunomide, teriflunomide, haloperidol, diazepam, risperidone, olanzapine, amisulpride, aripiprazole, asenapine, clopazine, iloperidone, lurasidone, paliperidone, quetiapine, ziprasidone, bupivacaine, lidocaine, ropivacaine, naltrexone, fentanyl, buprenorphine, butorphanol, loperamide, fingolimod, and salts, complexes, prodrugs, and analogs thereof.

One suitable drug that may be utilized in the present invention is testosterone or an ester thereof, including but not limited to testosterone undecanoate, or TU (also known as testosterone undecylate), testosterone cypionate (or TC), testosterone propionate, testosterone enanthate, and testosterone busciclate. Testosterone undecanoate is an ester of the hormone testosterone used in androgen replacement therapy, primarily for the treatment of male hypogonadism. Testosterone cypionate and other esters of testosterone, as well as testosterone base drug can be used for similar or the same indications. Testosterone undecanoate as well as testosterone cypionate, or testosterone base drug, or other forms of testosterone, may also be used as a male contraceptive, or in transgender (female-to-male) hormone therapy.

In some embodiments of the present invention, the API can be a prodrug, such as, by way of non-limiting example, testosterone undecanoate (TU). Where the API is a prodrug, the drug may be, inter alia, a hydrophobic salt or covalently bound ester of the corresponding drug, or bound to the polymer itself. Providing a prodrug as the API may provide important advantages or benefits in certain applications; by way of non-limiting example, providing the API as a prodrug may improve the stability of the formulation (e.g. during storage or irradiation, or after delivery in vivo), delay the release of the active form of the drug, affect or modify the solubility of the drug in the formulation, and/or extend or otherwise modify the duration of action of the drug. Where the prodrug is a covalently bound ester of the corresponding drug, the ester is often hydrolyzed in vivo to the corresponding carboxylic acid, which is then removed to convert the drug to its active form. This mechanism may be particularly beneficial where a low burst release and/or low peak plasma concentration of the drug is desirable, as in the case, by way of non-limiting example, of TU. In some embodiments, a desired release profile may be obtained by providing a mixture of a prodrug and the corresponding drug, in a predetermined ratio, as the API.

In some embodiments of the present invention, the API may be provided in crystalline form. In these embodiments, a selection of API crystal shape, or habit, may be another important consideration in the preparation of the LPT formulation, as different crystal habits may result in different release profiles. The selection of crystal habit will largely depend upon the API and desired release profile, but in general, the crystal habit should be stable throughout all manufacturing, shipping, and delivery conditions, e.g. during LPT formulation preparation, e-beam irradiation, shipping and storage, mixing, injection, etc. Additionally, different crystal habits may be more or less likely to form hydrates or polymorphs, which may be desirable or undesirable depending upon application, but it is generally advantageous that the transition into the hydrate or polymorph be predictable and/or controllable. Selection of a crystal habit can be based on these and other considerations. In some embodiments, the API is in a crystalline form having a block-like crystal habit or a needle-like crystal habit.

A desired particle size, or distribution of particle sizes, of the API will largely depend upon the API and the desired release profile. In general, a smaller particle size will result in more rapid release of the API in vivo (i.e., shorter duration of release) and/or a larger burst and corresponding higher peak concentration in vivo, while a larger particle size will result in slower release of the API in vivo (i.e., longer duration of release) and/or a smaller burst and corresponding lower peak concentration in vivo. Where the LPT formulation is an injectable formulation, the gauge of the needle used to inject the formulation may also be an important consideration in selecting a particle size, because large API particles may clog a large-gauge (i.e. small-diameter) needle or require excessive injection force. In some embodiments, a bimodal particle size distribution may provide an advantageous release profile or other desirable effect; by way of non-limiting example, and without wishing to be bound by any particular theory, it may be possible that smaller particles may cause rapid drug release (e.g. by faster release from a depot and/or faster solubilization upon release and/or modification of fluid channels in the depot) to provide an initial therapeutic effect, and larger particles may be released later to provide an extended therapeutic effect. Embodiments may also comprise particles of the API that have been encapsulated in, e.g., a microsphere or lipid sphere, which may provide an additional mechanism for controlling release of the API in vivo.

As used herein, unless otherwise specified, the term “particle size” refers to a median particle size determined by volume-based particle size measurements, such as, by way of non-limiting example, by use of a laser diffraction particle size analyzer such as a Malvern Mastersizer® instrument; such particle sizes may also be referred to as “D_{v,50}” values. Further, as used herein, unless otherwise specified, the term “span” refers to the difference between a 90th percentile particle size (referred to as “D_{v,90}”) and a 10th percentile particle size (referred to as “D_{v,10}”), divided by the 50th percentile particle size; thus, the span of a volume of particles can be interpreted as a measure of how broadly distributed particle sizes are within the volume. In various embodiments, the API will have a median particle size (D_{v,50}) of between about 10 μm and about 200 between about 10 μm and about 180 between about 10 μm and about 160 between about 10 μm and about 140 μm, between about 10 μm and about 120 μm, between about 10 μm and about 100 μm, between about 15 μm and about 100 μm, between about 15 μm and about 90 μm, between about 15 μm and about 80 μm, between about 20 μm and about 70 μm, between about 20 μm and about 60 μm, between about 25 μm and about 50 μm, between about 30 μm and about 90 μm, between about 40 μm and about 90 μm, between about 50 μm and about 90 μm, between about 60 μm and about 90 μm, or between about 70 μm and about 90 μm. In other embodiments, the median particle size of the active pharmaceutical agent in compositions of the invention can range from any whole number to any other whole number within the range of from about 1 μm and about 250 μm. Additionally, in various embodiments, the API may have a particle size span of between about 0.1 and about 8, or between about 0.5 and about 8, or between about 1 and about 8, or between about 1.5 and about 8, or between about 2 and about 7, or between about 3 and about 6, or between about 4 and about 5, or about 4.5, or between about 1.5 and about 5, or between about 1.5 and about 6, or between about 2 and about 6, or between about 2 and about 5, or between about 2 and about 4, or about 3, or alternatively about any tenth of a whole number between about 1 and about 8.

Yet another consideration in the preparation of LPT formulations according to the present invention is the choice of milling techniques used to prepare the API. Such techniques include, by way of non-limiting example, ball milling, cryomilling, cutter milling, homogenization, jet milling (also known as fluid energy milling), mortar-and-pestle grinding, nano-milling or wet milling followed by lyophilization or filtration or drying, roller milling, or runner milling. In many embodiments, jet milling will be the most desirable of these techniques due to its temperature control, reduced risk of contamination, and scalability, but techniques may be selected from among these and others based on the needs of a given application. As is described in further detail in the Examples, the present inventors have found that jet milling, also known as fluid energy milling, is an advantageous milling technique for providing API particles of a desired size. Among the benefits of jet milling in LPT formulations are (1) reduced risk of contamination with the milling medium, because the API comes into contact only with nitrogen gas; (2) low heat generation, which helps to keep the temperature of API particles below their melting point during milling; and (3) scalability to produce large quantities of API particles. The present inventors have also investigated nano-milling in water followed by lyophilization, and while this method may be used, it is less advantageous than jet milling for several reasons: the requirement for multiple pieces of equipment, the addition of wetting surfactants that may contaminate the API after lyophilization, the inclusion of residual water that may adversely affect polymer stability, etc. The present inventors have further investigated homogenization as a method for controlling particle size, and have found that it is less desirable than jet milling as the sole technique for particle size control, due to its relatively high heat generation and its inability to reduce particle size below an asymptotic limit, but may be desirable in combination with jet milling or other techniques because it is effective to break up clumps of the API and improves homogeneity of the resulting suspension. In general, the present inventors have found that mechanical micronization and milling techniques are generally more suitable than recrystallization techniques in LPT-TU formulations, as recrystallization risks introducing residual solvents and co-crystals that may affect formulation behavior and safety.

The concentration of active pharmaceutical agent in compositions of the invention depends on the drug that is included in the composition and may range from 0.1% to 50% by weight of the composition or higher. Typically, the concentration of agent in the composition is between 10% and 50% by weight of the composition, such as between 15% and 45% by weight of the composition, between 15% and 35% by weight of the composition, between 15% and 25% by weight of the composition, between 20% and 40% by weight of the composition, between 25% and 35% by weight of the composition, about 10% by weight of the composition, about 15% by weight of the composition, about 20% by weight of the composition, about 25% by weight of the composition, or about 30% by weight of the composition. In other embodiments, the amount of active pharmaceutical agent in compositions of the invention can range from any whole number percent to any other whole number percent within the range of from about 1 percent to about 50 percent by weight. In some embodiments, the concentration of the active pharmaceutical agent is no more than about 25% by weight.

Because a beneficial characteristic of the compositions disclosed herein is improved extended release of an active pharmaceutical agent, the amount of active pharmaceutical agent should be suitable for long term treatment with the agent in accordance with the time frames disclosed herein. Other embodiments of the invention include single dosage formulations of the liquid polymer pharmaceutical composition which include the liquid polymer composition as described herein with an amount of an active pharmaceutical agent suitable for extended release. For example, such single dosage formulations can include sufficient active pharmaceutical agent for treatment of a patient for at least three days, at least one week, at least two weeks, at least three weeks, at least four weeks, at least one month, at least two months, at least three months, at least four months, at least five months, at least six months, at least nine months, or at least one year. Compositions may be administered repeatedly as needed (e.g. every week, every 2 weeks, every month, every two months, every three months, every four months, every five months, every six months, etc.). Other dosage patterns are also suitable for use with the formulations of the invention, such as alternating dosing patterns (e.g., at Day 0, at 2 weeks, and then every month thereafter; or at Day 0, at 1 month and then every 3 months thereafter, etc.).

When the API used in a liquid polymer formulation of the invention is TU or TC (or another testosterone ester or testosterone base), the amount of TU or TC (or another testosterone ester or testosterone base) to administer to a subject should be sufficient to achieve the desired therapeutic effect, e.g., to provide testosterone supplementation in the eugonadal range (10.4-34.7 nmol/L or 3-10 ng/mL testosterone in plasma (see, e.g., Basaria or Morgentaler et al., supra); to treat or reduce the symptoms of androgen deficiency; to treat or reduce the symptoms of male hypergonadism; as an adjunct therapy for transgender men or gender reassignment; or as birth control. For example, as previously known and described in the art, testosterone undecanoate, when administered as an oil-based solution of the prior art, may be administered to males over 18 years of age as an initial 750 mg, 3 mL intramuscular dose, followed by another 750 mg, 3 mL intramuscular dose after four weeks and further 750 mg, 3 mL intramuscular doses every ten weeks thereafter. Alternatively, such a solution can be administered in a 1000 mg dose once every 12 weeks with no loading dose.

According to embodiments of the present invention, testosterone undecanoate (or testosterone or another testosterone ester, including, but not limited to, testosterone cypionate, testosterone enanthanate, or testosterone proprionate), when administered in the LPT formulations of the present invention, may be administered to a patient as a dose of between about 25 mg and about 1000 mg, or between about 100 mg and 1000 mg, or between about 150 mg and 1000 mg, or between about 200 mg and 1000 mg, or between about 250 mg and 1000 mg, or between about 500 mg and 1000 mg, or between about 750 mg and 1000 mg, or alternatively any whole number of milligrams between about 25 mg and about 1000 mg, including, but not limited to 100 mg, 150 mg, 200 mg, 250 mg, 300 mg, 350 mg, 400 mg, 450 mg, 500 mg, 550 mg, 600 mg, 650 mg, 700 mg, 750 mg, or higher. The amount of testosterone undecanoate (or testosterone or another testosterone ester), when administered in the LPT formulations of the present invention, can be sufficient to provide the desired therapeutic effect when administered weekly, biweekly, monthly, every two months, every three months, every four months, every five months, or every six months, every seven months, every eight months, every nine months, every ten months, every eleven months, or every twelve months, and for as long as testosterone supplementation is required. Testosterone undecanoate (or testosterone or another testosterone ester) can be provided in an LPT formulation of the invention in an amount sufficient to provide one or more initial loading doses at shorter intervals (e.g., weekly, biweekly or monthly), followed by maintenance doses, where the amount of API provided or the interval of the dosing increases, or under any alternative dosing regimen, such as by administering an initial larger dose followed by smaller maintenance doses, or by altering larger and smaller doses.

Additionally or alternatively, testosterone undecanoate (or testosterone or another testosterone ester), when administered in the LPT formulations of the present invention, may be administered at times and in amounts sufficient to achieve a serum testosterone concentration of between about 0.5 ng/mL and about 20 ng/mL, or between about 1 ng/mL and about 15 ng/mL, or between about 2 ng/mL and about 15 ng/mL, or between about 3 ng/mL and about 10 ng/mL, or between about 4 ng/mL and about 9 ng/mL, or between about 5 ng/mL and about 8 ng/mL, or between about 6 ng/mL and about 7 ng/mL, or about 6.5 ng/mL.

### Biocompatible Solvents for Use in the Invention

LPT pharmaceutical formulations according to the present invention comprise at least one biocompatible solvent. As noted above, in some embodiments the API may be substantially in solid form (i.e. solid particles of the API are suspended in the liquid polymer/solvent composition), while in other embodiments the API may be substantially or fully dissolved in the liquid polymer/solvent composition. As used herein unless otherwise noted, use of the term “suspension” when referring to a composition of the invention may refer to formulations in which at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, at least about 80%, or at least about 85%, or at least about 90%, or at least about 95%, or at least about 96%, or at least about 97%, or at least about 98%, or at least about 99% of the API is in the form of solid particles suspended in the liquid polymer and solvent composition. Description of an API herein as being “substantially in solid form” or “substantially in suspension” in a formulation refers to formulations in which at least about 50%, or at least about 55%, or at least about 60%, or at least about 65%, or at least about 70%, or at least about 75%, at least about 80%, or at least about 85%, or at least about 90%, or at least about 95%, or at least about 96%, or at least about 97%, or at least about 98%, or at least about 99% of the API is in the form of solid particles suspended in the liquid polymer and solvent composition.

As used herein unless otherwise noted, use of the term “solution” or description of an API as being “dissolved” in a formulation, refers to formulations in which at least 99% of the API is dissolved in the liquid polymer/solvent composition.

Solvents and co-solvents suitable for use in embodiments of the present invention include, by way of non-limiting example, acetone, benzyl benzoate, butyrolactone, ε-caprolactone, N-cycylohexyl-2-pyrrolidone, diethylene glycol monomethyl ether, dimethyl acetamide, dimethyl formamide, dimethyl sulfoxide (DMSO), ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, N-methyl-2-pyrrolidone (NMP), low-molecular weight (MW) polyethylene glycol (PEG), polysorbate 80, polysorbate 60, polysorbate 40, polysorbate 20, polyoxyl 35 hydrogenated castor oil, polyoxyl 40 hydrogenated castor oil, sorbitan monolaurate, sorbitan monostearate, sorbitan monooleate, benzyl alcohol, isopropanol, tert-butanol, n-propanol, propylene glycol, 2-pyrrolidone, α-tocopherol, triacetin, tributyl citrate, acetyl tributyl citrate, acetyl triethyl citrate, triethyl citrate, esters thereof, and combinations thereof. One or more of these and other solvents, including but not limited to benzyl benzoate, may form a suspension when provided in relatively small quantities and/or when used as a co-solvent or additive, and a solution when provided in relatively large quantities.

The solvent system used in an LPT formulation of the invention may comprise a combination or mixture of two or more compounds or components, and in some embodiments, the combination may include NMP or DMSO in combination with another component such as low molecular weight PEG (e.g. PEG 300 or PEG 400). The additional component, which may be generally referred to as an additive or co-solvent, e.g. PEG (by way of non-limiting example), may have any one of several effects, including but not limited to a true solvent effect (i.e. the API dissolves or is suspended in the additional component) or an effect by which the additional component does not directly dissolve or suspend the API but improves the degree to which the API is dissolved or suspended in the other solvent(s) (e.g. the additional component acts as a co-solvent, miscibility aid, solubilizer, non-solvent, or surfactant).

In one embodiment, an LPT formulation of the invention comprises an additional component that is a solubilizer, which is useful for increasing the solubility of the API in the LPT formulation, particularly in vivo. The presence of such a solubilizer, without being bound by theory, will reduce likelihood that the API (which has relatively low solubility in aqueous media, and/or is relatively hydrophobic) will crystalize, particularly in vivo when the solvent system dissipates from the formulation. In one aspect, the solubilizer is selected to have a release profile in the LPT formulation similar to that of the API, so that the release of the API and solubilizer are somewhat synchronous. For example, if the API is in a fatty acid ester form, such as testosterone undecanoate, then one could select a solubilizer that is a sucrose ester of a medium chain fatty acid ester, such as sucrose laurate. Examples of solubilizers useful in the present invention include solubilizers useful for parenteral injection, and include, but are not limited to, surfactants and other solubilizers, such as Poloxamer 188, sorbitan trioleate, lecithin (e.g., soya or egg), D-α-tocopherol polyethylene glycol succinate (e.g., Vitamin E TPGS), sugar-based esters or ethers (e.g., sugar acid esters of fatty alcohols or sugar alcohol esters of fatty acids, including, but not limited to, sucrose cocoate, sucrose stearate, sucrose laurate, etc,), amino acid-based solubility enhancers (e.g., proline, arginine, DL-methionine), protein-based solubility enhancers (e.g., hydrophobins) and combinations thereof. The use of sugar-based esters and ethers as solubilizers in parenteral pharmaceutical formulations is described, for example, in U.S. Pat. No. 8,541,360, which is incorporated herein by reference in its entirety.

When present, the additional component, e.g. PEG, may, in embodiments, be provided in any amount between about 15 wt % and 45 wt %, or between about 17 wt % and about 33 wt %, or between about 19 wt % and about 31 wt %, or between about 21 wt % and about 29 wt %, or between about 23 wt % and about 27 wt % of the formulation, or alternatively as any whole number percentage by weight of the formulation between about 15 wt % and about 45 wt %. In some embodiments the additional component may be relatively miscible with one or more other solvent(s), while in other embodiments the additional component may be relatively immiscible with one or more other solvents.

In embodiments of the present invention, the solvent, or combination or mixture of solvents and/or co-solvents, will generally comprise between about 20 wt % and about 95 wt % of the formulation, or between about 35 wt % and about 80 wt % of the formulation, or between about 45 wt % and about 65 wt % of the formulation, or between about 45 wt % and about 55 wt % of the formulation, or about 50 wt % of the formulation, or alternatively the solvent or combination or mixture of solvents and/or co-solvents can range from any whole number percentage by weight of the formulation to any other whole number percentage by weight of the formulation between about 20 wt % and about 95 wt %. Where the solvent comprises two or more compounds, any two compounds may be present in any weight ratio between about 99:1 and about 1:99, or between about 90:10 and about 10:90, or between about 80:20 and about 20:80, or between about 30:70 and about 70:30, or between about 40:60 and about 60:40, or about 50:50, or alternatively in any weight ratio X:Y where each of X and Y is a whole number between about 1 and about 99 and the sum of X and Y is 100.

In one embodiment, the solvent can be benzyl benzoate, in an amount between about 50 wt % and about 75 wt % of the formulation, or between about 55 wt % and about 70 wt % of the formulation, or between about 60 wt % and about 65 wt % of the formulation, or about 65 wt % of the formulation.

In another embodiment, the solvent can be a mixture of DMSO and low molecular weight PEG (e.g., PEG having a molecular weight of about 400 daltons), where the DMSO is included in an amount between about 15 wt % and about 55 wt % of the formulation, or between about 20 wt % and about 50 wt %, or between about 25 wt % and about 45 wt %, or between about 30 wt % and about 40 wt %; or at about 35 wt % of the formulation; and where the PEG is included in an amount between about 5 wt % and about 35 wt % of the formulation, or between about 10 wt % and about 20 wt %, or about 15 wt % of the formulation.

In yet another embodiment, the solvent may be a mixture of NMP and low molecular weight PEG (e.g., PEG having a molecular weight of about 300 daltons), where the NMP is included in an amount between about 15 wt % and about 50 wt % of the formulation, or between about 20 wt % and about 30 wt %, or about 25 wt % of the formuation, and where the PEG is included in an amount between about 15 wt % and about 35 wt % of the formulation, or between about 20 wt % and about 30 wt %, or about 25 wt % of the formulation.

Where the solvent is a combination or mixture of solvents, any two of the solvents in the mixture may be present in any weight ratio between about 1:99 and about 99:1. Where the solvent is a mixture of PEG and either NMP or DMSO, the ratio of PEG to NMP or DMSO is between about 20:80 and about 80:20, or between about 30:70 and about 70:30, or between about 35:65 and about 65:35, or about 50:50, or alternatively any ratio of whole numbers X and Y, where each of X and Y is at least about 1 and no more than about 99 and the sum of X and Y is 100.

For LPT formulations of the invention in which the API is not soluble or completely soluble in the formulation, the API is in suspension in the formulation rather than in a solution. In these embodiments, the sedimentation coefficient or rate of separation of the API and/or the polymer in the solvent system is advantageously on the order of days or weeks because this allows a user to mix the formulation to ensure homogeneity for minutes or hours, in some embodiments at least about 30 minutes, in advance of injection. In these and other embodiments, there may be no visually apparent separation of the API, the polymer, and/or the solvent from a remainder of the formulation for a period of at least about one month after initial suspension, or at least about two months after initial suspension, or at least about three months after initial suspension, or at least about four months after initial suspension, or at least about five months after initial suspension, or at least about six months after initial suspension.

### Biocompatible Liquid Polymers for Use in the Invention

The liquid polymer compositions of the invention comprise a biodegradable liquid polymer. In some embodiments, the polymers have a carboxylic acid end group, such as a glycolic acid end group, and may be made by standard chain-growth polymerization techniques, by combining one or more alkene or alicyclic monomers with a carboxylic acid or water, often a hydroxy acid, in the presence of a suitable catalyst, such as tin, for example in the form of stannous octanoate. Carboxylic acids that are suitable are those that contain an alkyl chain, a nucleophile, and are soluble in the monomer used to make the polymer or a combination of the monomer and solvent. Examples of suitable initiators include, but are not limited to, GHB (gamma-hydroxybutyric acid), lactic acid, glycolic acid, citric acid, and water. Typically, a biodegradable polymer with an acid end group is made by the ring opening polymerization of monomers, such as lactide and/or caprolactone, which is initiated by water or a carboxylic acid compound of the formula Nu-R—COOH where Nu is a nucleophilic moiety, such as an amine or hydroxyl, R is any organic moiety, and the —COOH is a carboxylic acid functionality. The nucleophilic moiety of the molecule acts to initiate the ring opening polymerization in the presence of a catalyst and heat, producing a polymer with a carboxylic acid functionality on one end. A representative polymerization equation is shown below as Formula A.

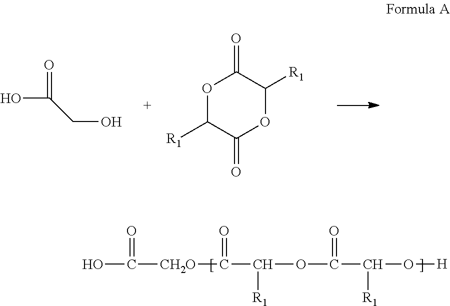

Alternatively, a carboxylic acid end group may be created on the end of a polymer chain by post-polymerization modification.

In addition to carboxylic acid end groups, liquid polymers according to the present invention may have any other suitable type of end group, including but not limited to ester end groups and hydroxyl end groups.

The liquid polymers that can be used according to the present invention are biodegradable, and remain in a liquid form, i.e. undergo continuous deformation under a shearing stress greater than zero and/or greater than a yield stress, at room temperature (e.g., at approximately 25° C.) up to body temperature (e.g., at approximately 37° C.), even after dissipation of the solvent from the polymer composition, such as when the polymer composition is exposed to an aqueous or largely aqueous environment (e.g. in vivo). The characteristic of being liquid is achieved by control of the molecular weight of the polymer and the monomer selection and ratio. In addition, the liquid polymer can have a pre-injection bulk viscosity that allows the composition to be easily administered, and in some embodiments effective to provide a desired controlled release profile of a biologically active agent from the implanted material. Because the liquid polymers are liquid at room and body temperature, they allow the use of lower concentrations of the biocompatible solvent to be used in the composition to provide a syringeable formulation compared to polymer/solvent compositions prepared with solid polymers.

Examples of suitable polymers that can be used in this application include polylactic acid, polyglycolic acid, polylactide (DL-lactide, D-lactide, L-lactide), polyglycolide, polycaprolactones, polyanhydrides, polyamides, polyurethanes, polyesteramides, polyorthoesters, polydioxanones, polyacetals, polyketals, polycarbonates, polyphosphazenes, polyhydroxybutyrates, polyhydroxyvalerates, polyalkylene oxalates, polyalkylene succinates, poly(malic acid), polyethylene glycol, hyaluronic acid, chitin and chitosan, and copolymers, terpolymers, and combinations or mixtures of the above materials. In one embodiment, the liquid polymer is selected from the group consisting of a polylactide, a polyglycolide, a polycaprolactone, a poly(trimethylene carbonate), a polydioxanone, a copolymer thereof, a terpolymer thereof, or any combination thereof. Suitable materials include, but are not limited to, those polymers, copolymers or terpolymers made with lactide, glycolide, caprolactone, p-dioxanone, trimethylene carbonate, 1,5-dioxepan-2-one, 1,4-dioxepan-2-one, ethylene oxide, propylene oxide, sebacic anhydride, diketene acetals/diols, and lactic acid with lower molecular weights and amorphous regions to limit crystallinity and subsequent solidification.

Non-limiting examples of suitable liquid polymers according to the invention include copolymers of DL-lactide and ε-caprolactone with molar ratios of lactide/caprolactone ranging from about 75/25 to about 25/75 and optionally with inherent viscosities as determined in a 0.10 g/dL solution of hexafluoroisopropanol (HFIP) at 25° C. from about 0.06 to about 0.38 dL/g, copolymers of caprolactone and 1,4-dioxanone with molar ratios of about 70/30 to about 40/60 and optionally with inherent viscosities of about 0.08 to about 0.24 dL/g, lactide and trimethylene carbonate copolymers such as 75/25 poly(DL-lactide-co-trimethylene carbonate), copolymers of caprolactone and trimethylene carbonate with molar ratios of about 90/10 to about 50/50 and optionally with inherent viscosities of about 0.09 to about 0.25 dL/g, and poly(L-lactic acid) optionally with an inherent viscosity of about 0.06 dL/g, among others. Generally, liquid polymers and liquid polymer compositions of the invention can have an inherent viscosity as determined in a 0.10 g/dL solution of hexafluoroisopropanol at 25° C. from 0.05 to 0.50 dL/g.

In embodiments of the composition, the biodegradable liquid polymer is a copolymer of two monomers having a molar ratio of any two whole numbers X to Y, where each of X and Y is at least about 25 and no more than about 75 and the sum of X and Y is 100. In one embodiment, the molar ratio of the two monomers in the copolymer is about 75/25.

In embodiments of the composition, a weight average molecular weight of the biodegradable liquid polymer is between about 1,000 daltons and about 25,000 daltons, or between about 5,000 daltons and about 25,000 daltons, or between about 6,000 daltons and about 24,000 daltons, or between about 7,000 daltons and about 23,000 daltons, or between about 8,000 daltons and about 22,000 daltons, or between about 9,000 daltons and about 21,000 daltons, or between about 10,000 daltons and about 20,000 daltons, or between about 11,000 daltons and about 19,000 daltons, or between about 12,000 daltons and about 18,000 daltons, or between about 13,000 daltons and about 17,000 daltons, or between about 14,000 daltons and about 16,000 daltons, or about 15,000 daltons. The weight average molecular weight of the biodegradable liquid polymer may be about 1,000 daltons, or about 2,000 daltons, or about 3,000 daltons, or about 4,000 daltons, about 5,000 daltons, or about 6,000 daltons, or about 7,000 daltons, or about 8,000 daltons, or about 9,000 daltons, or about 10,000 daltons, or about 11,000 daltons, or about 12,000 daltons, or about 13,000 daltons, or about 14,000 daltons, or about 15,000 daltons, or about 16,000 daltons, or about 17,000 daltons, or about 18,000 daltons, or about 19,000 daltons, or about 20,000 daltons, or about 21,000 daltons, or about 22,000 daltons, or about 23,000 daltons, or about 24,000 daltons, or about 25,000 daltons, or about 26,000 daltons, or about 27,000 daltons, or about 28,000 daltons, or about 29,000 daltons, or about 30,000 daltons, or about 31,000 daltons, or about 32,000 daltons, or about 33,000 daltons, or about 34,000 daltons, or about 35,000 daltons.

In one embodiment, the biodegradable liquid polymer can have a weight average molecule weight between about 1000 daltons and about 35,000 daltons. Alternatively, the weight average molecular weight of the biodegradable liquid polymer can be any whole number of daltons between about 1,000 daltons and about 25,000 daltons, or between about 1,000 and about 35,000 daltons.

In embodiments of the composition, the biodegradable liquid polymer may make up between about 0.1 wt % and about 50 wt % of the composition, or between about 5 wt % and about 45 wt % of the composition, or between about 10 wt % and about 40 wt % of the composition, or between about 15 wt % and about 35 wt % of the composition, or between about 20 wt % and about 30 wt % of the composition, or about 20 wt % of the composition, or about 25 wt % of the composition, or about 30 wt % of the composition. Alternatively, the biodegradable liquid polymer may make up any whole-number weight percentage of the formulation between about 1 wt % and about 50 wt %, or may make up a range from any whole-number weight percentage of the formulation to any other whole-number weight percentage of the formulation with end points between 1 wt % and 50 wt %.

The liquid polymers of the present invention can have a polydispersity value of from about 1.30 to about 2.50, or from about 1.35 to about 2.25, or from about 1.40 to about 2.00, or from about 1.45 to about 1.75, or about 1.50, or alternatively any twentieth of a whole number between about 1.30 and about 2.50.

Further examples of suitable liquid polymers of the invention include biodegradable liquid polymers with at least about 25% lactide (including DL-lactide) residues, at least about 30% lactide residues, at least about 35% lactide residues, at least about 40% lactide residues, at least about 45% lactide residues, at least about 50% lactide residues, at least about 55% lactide residues, at least about 60% lactide residues, at least about 65% lactide residues, at least about 70% lactide residues, or at least about 75% lactide residues. Other examples of suitable liquid polymers of the invention include biodegradable liquid polymers with residues of comonomers selected from caprolactone, trimethylene carbonate and combinations thereof in an amount at least about 5% and no more than about 75%, no more than about 70% such residues, no more than about 65% such residues, no more than about 60% such residues, no more than about 55% such residues, no more than about 50% such residues, no more than about 45% such residues, no more than about 40% such residues, no more than about 35% such residues, no more than about 30% such residues, or no more than about 25% such residues. Further embodiments include liquid polymers of 75:25 DL-lactide:ε-caprolactone, 75:25 DL-lactide:trimethylene carbonate, 25:75 DL-lactide:ε-caprolactone, and 75:25 ε-caprolactone:trimethylene carbonate.

The biodegradable liquid polymers of the invention can also be characterized as having at least one carboxylic acid end group. Further, the polymers can have a ratio of monomer units to carboxylic acid end groups that is between about 5:1 and about 90:1, between about 10:1 and about 90:1, between about 15:1 and about 90:1, between about 20:1 and about 90:1, between about 30:1 and about 80:1, between about 40:1 and about 70:1, between about 50:1 and about 60:1, or about 55:1. Alternatively, the ratio of monomer units to carboxylic acid end groups can be less than about 90:1, less than about 80:1, less than about 70:1, less than about 60:1, or less than about 55:1. The ratio of monomer units to carboxylic acid end groups can range from any whole number ratio to any other whole number ratio within the range of about 5:1 to about 90:1. The ratio of monomer units to carboxylic acid end groups corresponds to a number-average molecular weight of the polymer, which is equal to the weight-average molecular weight divided by the polydispersity index.

As is described in detail throughout this disclosure, various polymerization initiators can be selected when synthesizing the polymer for LPT formulations. The present inventors have generally found α-hydroxy acid initiators, and especially glycolic acid, to be advantageous for use in LPT formulations comprising testosterone or TU as the API.

### Liquid Polymer Compositions of the Invention

The liquid polymer compositions of the invention comprise a biodegradable liquid polymer, a biocompatible solvent or a combination or mixture of solvents or solvents and co-solvents, and an API, and are prepared by mixing or blending together the liquid polymer(s) and the solvent(s), which can be performed by any method at a temperature ranging from about 10-50° C. (e.g., at about 25° C.) using a suitable device to achieve a homogeneous, flowable liquid at room temperature. Examples of such devices include a mechanical stirrer, a mixer, or a roller mill. Because both the polymer and solvents are liquids, they may be mixed over a period (e.g., hours or days) to form a homogeneous solution or suspension.

In one embodiment, where the API is testosterone undecanoate (TU) or testosterone cypionate (TC), the formulation may be a suspension and may comprise between about 1 wt % and about 50 wt %, or between about 15 wt % and about 35 wt %, of NMP; between about 1 wt % and about 50 wt %, or between about 15 wt % and about 35 wt %, of low-MW PEG; between about 1 wt % and about 50 wt %, or between about 15 wt % and about 35 wt %, of TU or TC; and between about 1 wt % and about 50 wt %, or between about 15 wt % and about 35 wt %, of the biodegradable liquid polymer. The skilled artisan, in practicing the present invention, will select appropriate specific values for each component to ensure that the total amounts of all four components sum to 100 wt %.

In another embodiment, where the API is TU or TC, the formulation may be a suspension and may comprise between about 1 wt % and about 50 wt %, or between about 15 wt % and about 35 wt %, of DMSO; between about 1 wt % and about 50 wt %, or between about 15 wt % and about 35 wt %, of low-molecular weight PEG; between about 1 wt % and about 50 wt %, or between about 15 wt % and about 35 wt %, of TU or TC; and between about 1 wt % and about 50 wt %, or between about 15 wt % and about 35 wt %, of the biodegradable liquid polymer. The skilled artisan, in practicing the present invention, will select appropriate specific values for each component to ensure that the total amounts of all four components sum to 100 wt %.

In another embodiment, where the API is TU or TC, the formulation may be a solution and may comprise between about 40 wt % and about 90 wt %, or between about 55 wt % and about 75 wt %, of benzyl benzoate; between about 1 wt % and about 50 wt %, or between about 5 wt % and about 25 wt %, of TU or TC; and between about 1 wt % and about 50 wt %, or between about 10 wt % and about 30 wt %, of the biodegradable liquid polymer. The skilled artisan, in practicing the present invention, will select appropriate specific values for each component to ensure that the total amounts of all three components sum to 100 wt %.

In addition to particle size, the viscosity of the LPT formulation significantly affects the injection force necessary to administer the formulation. In general, it is desirable, for the purposes of patient comfort, to provide injections to the patient using the largest needle gauge (i.e. smallest needle diameter) possible, but a smaller needle requires a greater injection force to inject the formulation. Accordingly, needle gauge and injection force must be balanced against each other, and LPT formulations according to the present invention must have a combination of viscosity, particle size, and other factors that enables the formulation to be delivered down an appropriately large-gauge (i.e. small-diameter) needle by applying an appropriately moderate injection force. To minimize pain to the patient, it is desirable that LPT formulations according to the present invention be delivered using at most a 16-gauge needle (inner diameter 1.194 mm), or at least an 18-gauge needle (inner diameter 0.838 mm), or at least a 20-gauge needle (inner diameter 0.603 mm). The average adult male human can generate 85 newtons of force in a pinching motion using the thumb and forefingers, while the average adult female human can generate about 48 newtons of force by the same motion; it is thus desirable to provide LPT formulations having an injection force of no more than about 48 newtons, to enable the formulation to be administered by both men and women. Thus, by way of non-limiting example, where the formulation is to be delivered using a 20-gauge needle or by devices allowing similar injection forces, to ensure that the formulation can be delivered by an appropriate injection force, it may be advantageous that LPT formulations according to the present invention have a viscosity at room temperature of no more than about 5,000 cP, or no more than about 4,500 cP, or no more than about 4,000 cP, or no more than about 3,500 cP, or no more than about 3,000 cP, or no more than about 2,500 cP, or no more than about 2,000 cP. Other viscosity ranges may be advantageous for use with needles of other sizes.

LPT formulations of the present invention may be used for controlled-release delivery of the API, and as a result may have very high viscosities in vivo after dissipation of the solvent. By way of non-limiting example, the LPT formulations of the present invention may have viscosities under in vivo conditions, e.g. at about 37° C., of at least about 500 cP, or at least about 600 cP, or at least about 700 cP, or at least about 800 cP, or at least about 900 cP, or at least about 1,000 cP, or at least about 2,000 cP, or at least about 3,000 cP, or at least about 4,000 cP, or at least about 5,000 cP, or at least about 10,000 cP, or at least about 15,000 cP, or at least about 20,000 cP, or at least about 25,000 cP, or at least about 30,000 cP, or at least about 35,000 cP, or at least about 40,000 cP, or at least about 45,000 cP, or at least about 50,000 cP. Solvents may be selected to provide a suitably low viscosity prior to administration and a suitably high viscosity after administration; by way of non-limiting example, LPT formulations may be injectable as a thin, relatively inviscid (having no or negligible viscosity) liquid and then greatly increase in viscosity in vivo after injection. As a result of these very high in vivo viscosities and other factors, including but not limited to polymer molecular weight, solvent, and particle size and shape, LPT formulations of the present invention may form a depot in vivo that persists in a patient's body; in some embodiments, the depot may persist in the patient's body longer than the release profile of the API remains in the therapeutic range. By way of non-limiting example, an LPT formulation according to the present invention may form a depot in vivo that provides the API in the therapeutic range for about three months but that persists in the patient's body for at least about five months. The depot may have an in vivo viscosity that is much higher than a viscosity of the LPT formulation; by way of non-limiting example, an in vivo viscosity of the depot may, in some embodiments, be at least about 5,000 cP.

In some embodiments, the LPT formulation may comprise an additive to improve injectability, referred to as an “injectability booster;” by way of non-limiting example, the additive may comprise an enzyme, e.g. collagenase or hyaluronidase, to inhibit the formation of aggregates.

In some embodiments, the LPT formulation may comprise an additive to assist with the degradation of the polymer in situ (e.g., in vivo) over time. For example, it may be desirable to include an additive that ensures that the polymer completely degrades by a particular time point after injection which is commensurate with or shortly after the complete release of the API from the polymer.

The present invention provides LPT formulations that remain stable for an extended length of time under refrigeration, e.g. 2-8° C., and/or formulations that remain stable for an extended length of time at room temperature, e.g., 15-25° C., where “stability” refers to one or more of (1) negligible or minimal change in polymer mass, (2) chemical and physical stability of the API in the formulation (e.g., negligible or minimal change in API content and/or particle size), and (3) solvent and/or co-solvent content. In embodiments, there may be no more than about 10%, or no more than about 9%, or no more than about 8%, or no more than about 7%, or no more than about 6%, or no more than about 5%, or no more than about 4%, or no more than about 3%, or no more than about 2%, or no more than about 1% change in the polymer molecular weight, or no more than about 5 kDa, or no more than about 4 kDa, or no more than about 3 kDa, or no more than about 2 kDa, or no more than about 1 kDa change in polymer molecular weight, at intended storage conditions over an intended shelf life or at accelerated storage conditions over an abbreviated duration, and/or an activity of the API may change by no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1% at intended storage conditions over an intended shelf life. In general, increased shelf stability is always desirable, and the shelf life of formulations of the present invention at 5° C. and/or at room temperature is, in embodiments, at least about three months, or at least about six months, or at least about nine months, or at least about twelve months, or at least about fifteen months, or at least about eighteen months, or at least about 21 months, or at least about 24 months. In some embodiments, the LPT formulation may be suitable to be refrigerated for a longer period of time and then stored at room temperature for a shorter period of time for user convenience; by way of non-limiting example, LPT formulations according to the present invention may be shelf-stable at 5° C. for at least about 24 months, and then shelf-stable for an additional time of at least about one month at room temperature.

LPT formulations according to the present invention may be administered intramuscularly or subcutaneously to provide a systemic effect, or they may be administered by other means to provide a local effect of the API. By way of non-limiting example, LPT formulations may be administered in an articular region, a cutaneous region, an ocular region, and/or a tumor site region where it is desired that the API act locally rather than systemically. One advantage of LPT formulations of the invention is that, due to the liquid nature of the formulation, they form an implant or depot that can be characterized as soft, malleable, non-rigid, and/or non-solid. Such formulations, when administered into, for example, an articular region or other region where mobility or sensitivity may be an issue, result in the formation of an implant or depot in vivo after administration that has physical characteristics that allow the implant or depot to be better tolerated and have much less impact on mobility than, for example, a solid, hard implant.

Testosterone and esters thereof, especially TU or TC, are particularly suitable APIs for use in embodiments of the present invention. TU and/or TC in particular is a desirable API for use in the present invention because it requires or greatly benefits from sustained delivery when administered to treat male hypogonadism. As used herein, the term “LPT-TU formulation” refers to an LPT formulation comprising TU as the API. As used herein, the term “LPT-TC formulation” refers to an LPT formulation comprising TC as the API.

LPT-TU and LPT-TC formulations according to the present invention may be tailored to provide, as determined by the assays described in the Examples below, a desired in vitro release rate of TU or TC, respectively, which is useful for characterizing formulations and, in at least some cases, correlates with or may be informative of testosterone plasma concentration in vivo. Typically, the desired in vitro release rate for TU or TC is a prolonged, steady rate of release. It is also generally desirable that LPT-TU and LPT-TC formulations provide a low burst release (and therefore low peak concentrations in vivo) of TU or TC, respectively. In vitro release is highly dependent on the release conditions (e.g., buffer, surfactants, solvents, temp, etc). As is described in detail throughout this disclosure, various characteristics of an LPT-TU or LPT-TC formulation may be selected or optimized to provide a desired in vitro or in vivo release profile.

As is described in detail throughout this disclosure, the selection of a particle size distribution for testosterone or an ester thereof may be influenced by, inter alia, the desired release rate of the API and the gauge of the needle used to deliver the LPT formulation as an injection. By way of non-limiting example, a desired D_{v,50} particle size for TU in LPT-TU formulations or for TC in LPT-TC formulations, may be between about 15 μm and about 90 μm. For this reason, where an LPT-TU or LPT-TC formulation is to be administered by a 20-gauge needle, a D_{v,50} particle size of 250 μm or less may be desirable, but injectability must be balanced with the desired release profile. In general, reducing the particle size improves injectability but also increases the release rate and/or peak plasma concentration of the API; by way of non-limiting example, the present inventors have found that TU D_{v,50} particle sizes of about 15 μm are easily injectable, but may release too rapidly in vitro and, therefore, may result in a peak plasma concentration in vivo above the target range. Where an LPT-TU formulation is to be administered by a needle with a larger gauge (i.e. smaller diameter) than a 20-gauge needle, a D_{v,50} particle size less than 15 μm is therefore desirable. It is to be understood that formulations comprising pre-sized TU or TC that are then combined with a polymer and solvent(s) and then homogenized may have a different TU particle size in the combined formulation than in the original TU particulate starting material.

LPT-testosterone (including related salts, esters, complexes, prodrugs and analogs of testosterone) formulations according to the present invention generally provide testosterone supplementation in the eugonadal range, i.e. between about 10.4 and about 34.7 nmol/L, or between about 3 and about 10 ng/mL, in plasma. At least about 5%, and in some embodiments at least about 10%, and in some embodiments at least about 15%, and in some embodiments at least about 20%, and in some embodiments at least about 25%, and in some embodiments at least about 30%, and in some embodiments at least about 35%, and in some embodiments at least about 40%, and in some embodiments at least about 45%, and in some embodiments at least about 50%, and in some embodiments at least about 55%, and in some embodiments at least about 60%, and in some embodiments at least about 65%, and in some embodiments at least about 70%, and in some embodiments at least about 75%, of patients to whom LPT-testosterone or LPT-TU or LPT-TC formulations according to the present invention are administered will have total average testosterone concentrations in plasma between about 3 and about 10 ng/mL. To have therapeutic efficacy while minimizing side effects, LPT-testosterone and/or LPT-TU and/or LPT-TC formulations according to the present invention should provide a low burst release of testosterone, e.g. with no patients having a maximum testosterone concentration in plasma (Cₘₐₓ) of more than 25 ng/mL, or with no more than 5% of patients having Cₘₐₓ of more than 18 ng/mL, or with at least 85% of patients having Cₘₐₓ of no more than 15 ng/mL. LPT-testosterone and/or LPT-TU and/or LPT-TC formulations according to the present invention can maintain these therapeutic levels for extended periods of time as short as one week, and in some embodiments up to about twelve months, e.g. at least about one week, at least about two weeks, at least about three weeks, at least about one month, at least about two months, at least about three months, at least about four months, at least about five months, at least about six months, or at least about seven months, or at least about eight months, or at least about nine months, or at least about ten months, or at least about eleven months.

Embodiments of the invention include liquid polymer pharmaceutical compositions of testosterone or testosterone undecanoate (or other testosterone esters, including, but not limited to, or testosterone cypionate) and use thereof in the treatment of androgen deficiency, in particular male hypogonadism, by administration to a subject having androgen deficiency, such as a male having hypogonadism in amounts and dosing schedules described above and by routes of administration including but not limited to subcutaneous administration, intramuscular administration, and other forms of parenteral administration.

Various modifications of the above described invention will be evident to those skilled in the art. It is intended that such modifications are included within the scope of the following claims.

The invention is illustrated by the following non-limiting examples.

### EXAMPLES

### Example 1

The following example describes the preparation and test methods for Liquid Polymer Technology (LPT) formulations comprising testosterone undecanoate (TU) or testosterone cypionate (TC).

LPT Polymers To produce the formulations described in Examples 2-8 below, LPT polymers, which were glycolic acid-initiated, 75:25 poly(DL-lactide-co-ε-caprolactone) (PDLCL) liquid polymers (i.e., polymers comprised of 75% DL-lactide and 25% ε-caprolactone (mol:mol)), were produced using the following methods. Specifically, to produce a 75:25 poly(DL-lactide-ε-caprolactone) liquid copolymer, DL-lactide, ε-caprolactone, and glycolic acid (or other suitable acid initiator) were provided in an amount calculated to achieve the target molar composition and weight average molecular weight. Table 1 provides exemplary amounts of the monomers and acid initiators calculated to produce a 500 gram batch of copolymers having various target weight average molecular weights and used throughout the Examples. It is noted that the quantities of monomer and initiator shown in the table are illustrative, and the exact quantities of monomer and initiator may vary when different lots of monomer are used, or when different acid initiators are used, and can be calculated by one of skill in the art. Upon e-beam irradiation, it is noted that the weight average molecular weight of the polymer may reduce by approximately 0.1-20%, with higher molecular weight polymers typically experiencing a larger reduction within this range than lower molecular weight polymers; therefore, the desired molecular weight of the polymer in the final formulation (post-irradiation) may be different as compared to the initial molecular weight.

TABLE 1

| Weight Average | | DL-Lactide | | ε-Caprolactone | | Glycolic Acid | |
|---|---|---|---|---|---|---|---|
| Molecular Weight | | Grams | Mol | Grams | Mol | Grams | Mol |
| 5 | kDa | 316.5 | 2.2 | 83.5 | 0.73 | 32.0 | 0.42 |
| 10 | kDa | 396 | 2.7 | 104 | 0.91 | 14.5 | 0.19 |
| 14 | kDa | 194 | 1.4 | 52 | 0.46 | 3.6 | 0.047 |
| 15.5 | kDa | 475 | 3.3 | 125.3 | 1.1 | 8.9 | 0.12 |
| 18 | kDa | 395.6 | 2.74 | 104.4 | 0.91 | 5.25 | 0.7 |
| 22 | kDa | 396 | 2.7 | 104 | 0.91 | 5.05 | 0.66 |

To produce the polymers, a 500 mL 2-part glass reactor equipped with a nitrogen inlet, an overhead stirrer with a vacuum-capable stir guide and a vacuum outlet leading to a vacuum trap and vacuum pump was assembled and placed in an oil bath. The oil bath was set at 100° C. and the reactor was placed under vacuum to remove any residual moisture.

For each polymer composition, the vacuum on the reactor was broken with nitrogen and the reactor was charged with the prescribed amounts of DL-lactide, glycolic acid and ε-caprolactone via a glass funnel. The stirrer was turned to 10-50 rpm, the oil bath set to 160° C., and the system vacuum purged and back flushed with nitrogen three times. The reactor was then left under a slight nitrogen purge.

A catalyst solution was prepared by weighing the appropriate amount of tin(II) 2-ethylhexanaote (stannous octoate) into a 10 mL volumetric flask and diluting to the mark with anhydrous toluene. For all polymer compositions described in these Examples, once the monomers had melted and the oil bath reached 160° C., typically 5 mL of the catalyst solution was injected in an amount calculated to achieve 0.03 wt % catalyst solution based on the monomer weight via a syringe equipped with a 6-inch blunt tipped 20 g needle with continuous stirring. By way of example, for a 400 gm batch of polymer, the amount of catalyst solution needed to add 0.03 wt % stannous octoate based on monomer weight was calculated as 0.12 g (in 5 mL of toluene). For a 500 gm batch of polymer, the amount needed to add 0.03 wt % stannous octoate based on monomer weight was calculated as 0.15 g (in 5 mL of toluene).

After injection of the catalyst solution, the polymerization reaction was continued for 16-18 hours. After the appropriate reaction time, the vacuum trap was immersed in an ice bath and the nitrogen inlet closed. Vacuum was applied slowly to the stirred reaction mix for 4-6 hours with an ultimate vacuum of −22 to −25 in. Hg. Unreacted monomer was collected in the vacuum trap. After the appropriate time the vacuum was discontinued, the reactor purged with nitrogen, removed from the oil bath and the liquid polymer poured into a metal, glass or PYREX® (low-thermal-expansion plastic borosilicate glass) container and cooled. Yield was approximately 85% for all polymer compositions.

The weight average molecular weight of the polymers was determined by gel permeation chromatography (GPC) with a refractive index detector (e.g., Agilent 1260 Infinity Quaternary LC with Agilent G1362A Refractive Index Detector).

LPT Formulations To produce the LPT formulations comprising the active pharmaceutical ingredient (API), testosterone undecanoate (TU), used in Examples 2-7, or testosterone cypionate (TC), used in Example 8, 75:25 PDLCL LPT polymer of the indicated weight average molecular weight (see individual experiments below) was combined with the indicated solvent, and co-solvent (if included), and mechanically mixed to assist in the dissolution and/or dispersion of the solvent in the polymer.

Briefly, LPT polymer was heated to 60-115° C. (typically 80° C.) and dispensed into an appropriate container. After dispensing the LPT polymer, solvent(s) in the prescribed amounts were added to the same container. The container was mixed until a visually and tactilely (via interrogation with a metal spatula) homogeneous solution was formed. This mixing was performed on a 3-dimensional shaker/mixer (i.e., a TURBULA shaker mixer), at 40 rpm for greater than 48 hours, or on ajar mill with similar conditions. The LPT/solvent mixture was sometimes filtered after dissolution using a pressure pot and compatible filter. The resulting solution was a viscous, but flowable liquid polymer, which was at that point a drug-free polymer/solvent composition.

For the LPT-TU suspensions used in Examples 2-7, testosterone undecanoate (TU) was added to the polymer/solvent solution, and mixed into the polymer/solvent composition in the amounts required to achieve the desired percentages as indicated in the various experiments below, and mixed until homogenously dispersed. Preparation of LPT-TU solutions is described in Example 7 below. In some experiments, the TU/polymer/solvent mixture was homogenized to allow for injection through a 20 G needle. The average particle size (D_{v,50}) of the TU used to form these suspensions comprised a wide range of particle sizes. To form a suspension, the TU was mixed into the LPT/solvent and the TU dispersed and any aggregates/clumps were broken up, for example, by using an in-line homogenizer (e.g., IKA Magic Lab at 3,000 rpm for not less than 10 minutes) or a drop down homogenizer (e.g., a Silverson homogenizer at 2,000 to 3,500 rpm for not less than about 10 minutes). After incorporation of the TU into the polymer/solvent mixture, the formulation was filled into syringes and the syringes capped. The production of LPT-TU solution formulations is described in Example 7.

For the LPT-TC suspensions used in Example 8, testosterone cypionate (TC) was added to the polymer/solvent solution, and mixed into the polymer/solvent composition in the amounts required to achieve the desired percentages as indicated in the experiments in Example 8, and mixed until homogenously dispersed. More specifically, to form a suspension, the TC was mixed into the LPT/solvent and the TC dispersed. In some experiments, aggregates/clumps were broken up, for example, by using a drop down homogenizer (e.g., a Silverson homogenizer at 2,500 rpm for not less than about 10 minutes). After incorporation of the TC into the polymer/solvent mixture, the formulation was filled into syringes and the syringes were capped.

After production of the LPT-TU or LPT-TC formulations, filled syringes were stored under refrigerated conditions (e.g., 2-8° C.), and for in vivo studies, the syringes were irradiated via e-beam irradiation (or similar). For in vitro studies, e-beam irradiation was not required and was not always performed. Briefly, for e-beam irradiation, were packaged in a secondary container (e.g., a foil pouch or tray pack), typically with a desiccant/molecular sieve. A total irradiation dose of 30 kGy was administered to reach an approximate total internal dose of 25 kGy. In some experiments, an irradiation scheme of two passes at 15 kGy with a hold time of at least 1 hour at refrigerated conditions between passes was used to control sample temperature during irradiation.

Testosterone Undecanoate For the experiments conducted in Examples 2-7, testosterone undecanoate was processed to have a desired particle size distribution using one of the following methods: (1) homogenized after formulating with the polymer and solvent (Example 2); (2) homogenized or milled in water, then lyophilized and added to LPT formulations (Example 4, all TU samples except 86 μm TU); (3) dry-sieved then added to LPT formulations (Example 4, 86 μm TU); or (4) jet milled, which is a milling process that reduces the particle size of the drug by repeated impact events between particles, and then added to LPT formulations and homogenized to disperse (Examples 3, 5 and 6). The particle size was determined using number-based particle size calculation methods, volume-based particle size calculation methods, or both methods (see Table 2A).

In a number-based particle size distribution method, the particles were measured using microscopy, where a size was assigned to each particle inspected. This approach builds a number distribution, where each particle has equal weighting once the final distribution is calculated. D₁₀ is the value of the particle diameter at 10% in the cumulative distribution, D₅₀ is the value of the particle diameter at 50% in the cumulative distribution, and D₉₀ is the value of the particle diameter at 90% in the cumulative distribution. The number-based particle size values for TU in the experiments described herein were determined either prior to incorporation into the polymer/solvent, or after the TU is incorporated into the polymer/solvent and is then further homogenized, as indicated. Therefore, particle size values determined prior to incorporation into the formulation may be different, e.g., slightly higher, than those determined after incorporation into the formulation.

In a volume-based particle size distribution method, which is typically measured using laser diffraction analysis, the majority of the total particle mass or volume comes from the larger particles, and so the volume-based particle size distribution typically results in a larger median particle size number (D_{v,50}) as compared to number-based methods, simply on the basis of the distribution calculation. The D_{v,50} is known as the median or the medium average of the particle size distribution in a volume of particles; it is the particle diameter value at the median of the cumulative distribution, wherein 50% of the volume of the particle sample is comprised of particles having a larger diameter than this value and 50% of the volume of the particle sample is comprised of particles having a smaller diameter than this value. D_{v,10} is the particle diameter value at which 10% of the volume of the particle sample is comprised of smaller diameter particles, D_{v,90} is the particle diameter value at which 90% of the volume of the particle sample is comprised of smaller diameter particles. Volume-based particle distribution can be measured, for example, using a laser diffraction particle size analyzer, such as Mastersizer® (Malvern Panalytical, Malvern, Pa.). Software programs and calculations exist that are able to convert the results from a number-based distribution analysis to a volume-based distribution analysis and vice versa. Therefore, for particle sizes calculated using a number-based method, a volume-based particle size can also be estimated, and vice-versa. Volume-based particle size distribution measurements are the default choice for many ensemble light scattering techniques including laser diffraction, and are commonly used in the pharmaceutical industry (Burgess, J., Duffy, E., Etzler, F., Hickey, A., Particle Size Analysis: AAPS Workshop Report, Cosponsored by the Food and Drug Administration and the United States Pharmacopeia, AAPS Journal 2004; 6 (3) Article 20).

Table 2A shows the number-based particle size distribution (D₁₀, D₅₀, and D₉₀) and/or the volume-based particle size distribution (D_{v,10}, D_{v,50}, and D_{v,90}) for Test Formulations 1-25 used in the in vitro and in vivo experiments described in Examples 2-6. The particle size distribution values in Table 2A are provided for the TU prior to incorporation of the TU into the polymer and solvent (shown as TU API Particle Size Distribution (PSD)). Particle size distribution values can also be determined after the TU has been incorporated into the polymer/solvent and then further homogenized, which is the final formulation TU particle size.

Table 2B shows a comparison of four different test methods (labeled T1, T2, T3 and T4) which were used to calculate the volume-based particle size distribution of the Test Formulations described herein. For each test method, differences in various parameters of the protocol or equipment used are illustrated. It is understood by one skilled in the art that differences in particle size test methodology, including equipment make/model, settings, and sample preparation technique, can lead to variability in the resulting particle size determinations. This is illustrated in Table 2C, which illustrates the differences in Dv,50 values which have been obtained using the same material on the same instrument with variations in settings and test conditions, with reference to the test methods described in Table 2B. According to the present invention, a Dv,50 value can be determined by any of the methods described herein or otherwise known in the art and such values are within the scope of the claimed invention.

TABLE 2A

| | TU API Particle Size Distribution (PSD) Pre-Formulation | | | | | | |
|---|---|---|---|---|---|---|---|
| | | | | Mastersizer | | | |
| | Microscopic | | | (volume based) | | | |
| Test | (number based) | | | | | | Test Method |
| Formulation # | D₁₀ | D₅₀ | D₉₀ | D_{v, 10} | D_{v, 50} | D_{v, 90} | from Table 2B |
| 1 | — | — | — | 16 | 90 | 470 | T4 |
| 2, 11 | | | | | | | |
| 3 | | | | | | | |
| 4, 7, 8, 12, 14, 17 | 4 | 9 | 18 | 2 | 15 | 56 | T2 |
| 21 | | | | | | | |
| 22 | | | | | | | |
| 5, 13, 15, 19 | 8 | 20 | 55 | 9 | 64 | 162 | T1 |
| 6, 9, 10, 18 | 5 | 15 | 41 | 7 | 56 | 185 | T2 |
| 23 | | | | | | | |
| 16 | 1 | 2 | 4 | 1 | 6 | 21 | T1 |
| 20 | 7 | 18 | 40 | 19 | 86 | 183 | T4 |
| 24 | — | — | — | 4 | 34 | 173 | T2 |
| 25 | — | — | — | 12 | 63 | 255 | T3 |

TABLE 2B

| | | | | | | Instrument | Particle | |
|---|---|---|---|---|---|---|---|---|
| Test | Slurry | Slurry | Circulating | Bath | Instrument | Stir Rate | Refractive | Absorption |
| Method | Method | Media | Fluid | Sonication | Sonication | (rpm) | Index | Index |
| T1 | Rotate | 2% | 10 mM | 90 s | No | 1000 | 1.62 | 0.1 |
| | in tube | Tween 20 | NaCl | | | | | |
| | w/media | | | | | | | |
| T2 | Magnetic | 0.2% | 0.1% | 240 s | 120 s | 2500 | 1.465 | 0.01 |
| | stirring | Tween 80 | Na4P2O7 | | (80%) | | | |
| T3 | Magnetic | 0.2% | 0.1% | No | No | 2500 | 1.465 | 0.01 |
| | stirring | Tween 80 | Na4P2O7 | | | | | |
| T4 | Vortex | 0.2% | 0.1% | No | 120 s | 2500 | 1.465 | 0.01 |
| | | Tween 80 | Na4P2O7 | | (80%) | | | |

TABLE 2C

| | Tolmar Particle Size Analysis (Dv, 50 μm) | | | |
|---|---|---|---|---|
| Test Formulation # | T1 | T2 | T3 | T4 |
| 1, 2, 3, 11 | — | — | 172 | 90 |
| 4, 7, 8, 12, 14, 17, 21, 22 | 20 | 15 | — | — |
| 6, 9, 10, 18, 23 | 55 | 56 | — | 81 |

Testosterone Cypionate. For the experiments conducted in Example 8, testosterone cypionate was used as provided by the supplier (either Fabbrica Italiana Sineteici S.p.A. or Pfizer). Testosterone cypionate was provided having a volume-based particle size (Dv,50 μm), as determined by Malvern, of approximately 29 μm or approximately 41 μm.

Production of Non-Polymeric Testosterone Undecanoate Control Solution. For several of the Examples described herein, a non-polymeric testosterone undecanoate control solution (also referred to as the “non-polymeric control solution” or “non-polymeric TU control solution”) was used. To prepare this control solution, testosterone undecanoate (TU), benzyl benzoate (BzBz), and castor oil were combined in a 23.9/47.9/28.2 TU/BzBz/castor oil wt % ratio. The components were mixed on a TURBULA® shaker mixer (GlenMills, N.J.) until the TU was fully dissolved, at a mixing speed of −40 rpm for not less than 12 hours to achieve a visually homogeneous formulation. After full dissolution of the TU, the formulation was filtered via a 0.2 or 0.45 μm filter.

In vitro Release Testing. In order to evaluate testosterone undecanoate or testosterone cypionate release from the LPT-TU or LPT-TC samples in vitro, a sample containing approximately 40 mg TU or TC of each of the LPT-TU or LPT-TC formulations, respectively, was injected into a sample holder. The sample holders were then carefully placed into sample jars containing temperature-equilibrated (37° C.) aqueous release media (pH 8.7 50 mM TRIS, 50 mM ammonium sulfate, 1 wt % hexadecyltrimethylammonium bromide, also known as cetyltrimethylammonium bromide, or CTAB), and placed onto an incubated shaker (38.5° C. temperature setting and 25 rpm, increased to 90 rpm after 60+10 minutes). Samples of the in vitro release media were collected at specified timepoints (e.g., 1 hour (0.04 days), 3 hours (0.13 days), 6 hours (0.25 days), 11 hours (0.46 days), 1 day, 2 days, 4 days, 7 days, 10 days, 14 days, and 21 days), and each sample was analyzed by high performance liquid chromatography (HPLC) for testosterone undecanoate or testosterone cypionate content. Both the release rate (mg/day) and percent cumulative release (%) of testosterone undecanoate or testosterone cypionate were calculated for each time point.

Evaluation of Data For the in vitro and/or in vivo experiments described herein, a target range, or target window, of TU or TC release (in vitro experiments) or plasma testosterone levels (in vivo experiments) may be referenced. For the in vitro experiments, a target window for TU release was generally defined using a lower release rate set at −1.1 mg/day, and an upper release rate set at −3.6 mg/day (resulting in a median of −2.35 mg/day). This target range was established for purposes of general evaluation and comparison of formulations. The target in vitro values are based on qualitative correlation between the in vitro release rate of TU and the in vivo testosterone concentration in plasma, when the same LPT-TU formulations were tested under in vitro and in vivo conditions, as well as by qualitative agreementwith an in vivo plasma testosterone target window in rats (3-10 ng/mL), such as that used in the animal PK studies described herein (Examples 2 and 5-7). These limits are based on the target of 3-10 ng/mL in rats and may or may not apply to target ranges in other animals or humans. Alternate in vitro target windows or alternate release conditions (media, temperature, sample collection times etc.) may be better suited when considering other animal models or humans. These values are noted only for general information and evaluation purposes in the in vitro assays and are not necessarily reflected in the Figures. The target window for TC release is generally defined in a similar manner, although the lower and upper limits can vary somewhat. As with TU, the target range for TC release was established for purposes of general evaluation and comparison of formulations. Moreover, it is not required that an LPT-TU formulation release TU solely within this range or that an LPT-TC formulation release TC solely within this range to be a suitable formulation according to the present invention. For example, LPT-TU formulations that release TU more quickly have a maximum in vitro release rate (RRₘₐₓ) that is above the 3.6 mg/day mark, and/or drop below 1.1 mg/day more quickly than other formulations may be candidates for formulations in which a shorter duration of activity is desired and/or in which an increased rate of drug release is desired. The same is true for LPT-TC formulations. Such LPT formulations may be also useful with a different API having physical characteristics similar to TU (e.g., an API with low solubility in an aqueous environment), and where a condition associated with such API can be treated, or should be treated, using a formulation with a shorter duration of action, or where a higher Cₘₐₓ is well-tolerated or even desirable.

For the animal PK studies described herein, a target range, or target window, of mean plasma testosterone concentration levels was set at between 3 and 10 ng/mL, which is approximately equivalent to 10.4-34.7 nmol/L testosterone in plasma, and which is based on FDA guidelines for humans, corresponding to testosterone supplementation in the eugonadal range e.g. (see, e.g., Basaria and Morgentaler). It should be noted that the target window is based on acceptable human levels, and use in the alternate species in the in vivo experiments was intended as a criteria to evaluate and compare formulations. However, LPT formulations that result in mean testosterone concentrations above or below this window, for shorter or longer durations, such as formulations that have a Cₘₐₓ above and/or below this target range, or a concentration above and/or below this range for any portion of the experiment, are still considered to be useful LPT formulations and illustrative of particular embodiments of the invention. Some formulations may have a shorter duration of action, and these formulations may be useful, for example, when such a shorter active window is desired, or when using a different API having physical characteristics similar to TU, but where a condition associated with such API can be treated, or should be treated, using a formulation with a shorter duration of action. As another example, formulations that remain within the target window for a longer period of time are candidates for formulations in which a longer and more sustained duration of activity is desired. Some formulations may have a Cₘₐₓ above this target range, and these formulations may be useful, for example, or where a higher Cₘₐₓ is well-tolerated or even desirable. Some formulations may have a Cₘₐₓ below this target range, and these formulations may be useful, for example, or where a lower Cₘₐₓ is efficacious or even desirable, possibly due to adverse effects at high Cₘₐₓ. Some formulations may have drug concentrations above or below this target range, and these formulations may be useful, for example, or where a higher or lower dose is well-tolerated or even more efficacious or desirable.

### Example 2

The following example provides experimental results directed toward the development of Liquid Polymer Technology (LPT) formulations comprising testosterone undecanoate (TU) in the form of a suspension.

To produce an LPT formulation with favorable drug release kinetics and depot degradation, extended release capability (e.g., 60-90 days), and stability within target temperature ranges and time periods, LPT formulations having different molecular weight polymers and/or solvents were produced as described in Example 1 and are shown in Table 3. Table 3: (1) lists the composition of each of the LPT Test Formulations with respect to the percentage by weight of: testosterone undecanoate (TU), LPT polymer (LPT), solvent (Sol), and co-solvent (Co-Sol); (2) provides the TU particle size (volume-based, D_{v,50}); (3) indicates the LPT polymer and weight average molecular weight (Polymer, MW) of the polymer; (4) identifies the solvent and co-solvent (if any); (5) describes the physical form of the testosterone undecanoate in the post-e-beam (final) formulation; (6) and provides the viscosity of the final LPT formulation, post-e-beam irradiation.

LPT-TU Test Formulations 1-3 (Table 3) were selected for analysis based on the preliminary evaluation of over 75 different LPT formulations, because each of these formulations has the following characteristics: (1) does not freeze at refrigeration temperatures (-2-8° C.); (2) is thermally stable at e-beam temperatures of 20-45° C. (i.e., TU does not substantially change physical state in the formulation and remains in suspension or substantially solid form); and (3) the final formulation is a suspension. The control formulation (X) in this experiment is also an LPT-TU suspension formulation that does not freeze at refrigeration temperatures; however, it is not thermally stable at e-beam temperatures; i.e., the TU dissolves into the polymer and solvent matrix at the higher temperatures encountered during e-beam irradiation, and then recrystallizes upon cooling to form a recrystallized suspension, which results in a formulation having less favorable testosterone release kinetics in vivo.

TABLE 3

| | Test | | | | | | |
|---|---|---|---|---|---|---|---|
| | Formulation | TU Particle Size | | | | Formulation | |
| | TU/LPT/Sol/Co-Sol | (Volume Based | Polymer | | Co- | Physical | Viscosity |
| # | (by weight %) | D_{v, 50}) | (MW) | Solvent | Solvent | State | (cP) |
| X | 20/30/50/0 | N/A | 75:25 PDLCL | NMP | — | Recrystallized | 1576 |
| | | (Recrystallized) | (22 kDa) | | | Suspension | |
| 1 | 20/30/25/25 | 90 μm | 75:25 PDLCL | NMP | PEG 300 | Homogenized | 1100 |
| | | | (5 kDa) | | | Suspension | |
| 2 | 20/30/25/25 | 90 μm | 75:25 PDLCL | NMP | PEG 300 | Homogenized | 2070 |
| | | | (14 kDa) | | | Suspension | |
| 3 | 20/30/35/15 | 90 μm | 75:25 PDLCL | DMSO | PEG 400 | Homogenized | 840 |
| | | | (5 kDa) | | | Suspension | |

PDLCL = poly(DL-lactide-co-ε-caprolactone) liquid polymer
NMP = N-Methyl-2-Pyrrolidone
DMSO = Dimethyl Sulfoxide
PEG 300 = Polyethylene glycol, 300 Da
PEG 400 = Polyethylene glycol, 400 Da

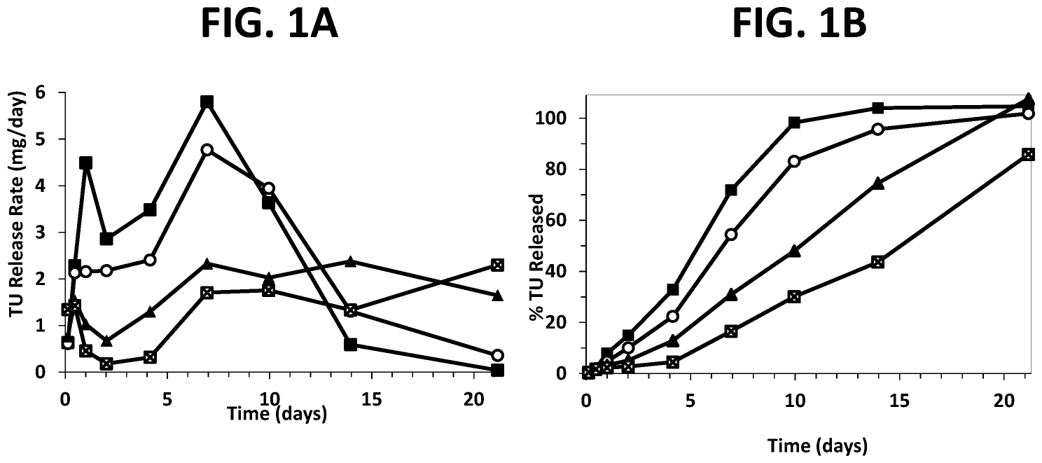

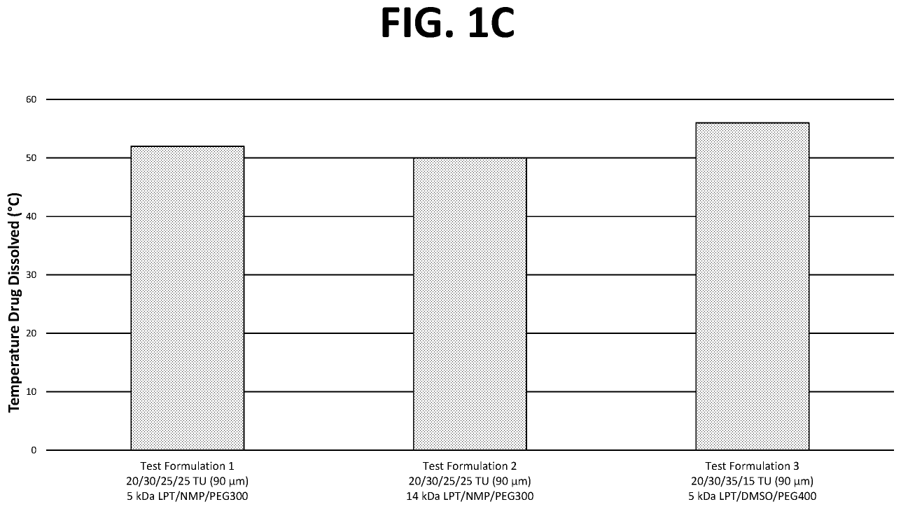

Test Formulations 1-3 and the Control Formulation X as shown in Table 3 were produced as described in Example 1 and were evaluated in an in vitro TU release test also as described in Example 1. FIGS. 1A and 1B show the results of the in vitro release testing of Test Formulations 1-3 (Test Formulations 1 (▪), 2 (▴) and 3 (∘)) and the Control (X) LPT Formulation. FIG. 1A shows the TU release rate (mg/day) and FIG. 1B shows the percentage of TU released over time (days).

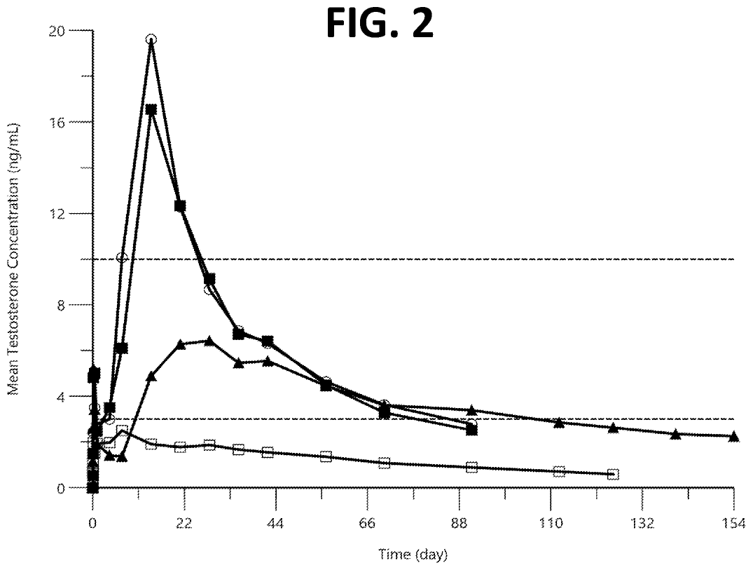

FIGS. 1A and 1B show that, as compared to the Control LPT Formulation (X), all three LPT Test Formulations released TU more rapidly, and FIG. 1B shows that all three formulations achieved 100% release by Day 21 of the experiment. The two formulations comprising the lower molecular weight polymer (5 kDa, Test Formulations 1 and 3, ▪ and ∘, respectively) had a faster rate of TU release and reached maximum TU release much earlier than the formulation comprising the 14 kDa polymer (Test Formulation 2, ▴). The Control LPT Formulation (X) did not achieve 100% release by Day 21. FIG. 1A is an alternative representation of in vitro release data, where the rate of TU release is plotted against time. This representation of TU in vitro release data correlates qualitatively with the in vivo testosterone plasma concentration versus time for the same LPT-TU formulations (see FIG. 2, discussed below). FIG. 1A shows that the maximum in vitro release rate (“RRₘₐₓ”) is much higher for Test Formulations 1 and 3 than for Test Formulation 2. As used herein, reference to RRₘₐₓ, or maximum release rate, in an in vitro test, refers to the maximum (peak) in vitro release rate from the formulation. A similar measurement is used in in vivo tests, which is called Cₘₐₓ. Reference to “Cₘₐₓ” typically refers to a pharmacokinetic measurement of rate that is the maximum (peak) serum concentration of the drug achieved after a dose of the drug is given, and is typically used in in vivo studies.

One can also review in vitro data such as that shown in FIGS. 1A and 1B and elsewhere herein by referring to the T50%, which is the time it takes to achieve 50% drug release (e.g., with reference to FIG. 1A, one can calculate the time (day) at which 50% of drug was released, which is a useful additional comparison, particularly when one formulation releases drug much more quickly and reaches 100% release much earlier, as compared to a slower releasing formulation. FIG. 1A also shows that the TU release rate decreases more quickly for Test Formulations 1 and 3 than for Test Formulation 2 after reaching the point of maximum release rate. The Control LPT Formulation released TU more slowly than the Test Formulations for the first half of the study. These same trends for Test Formulations 1, 2, and 3 were observed in vivo (see below, FIG. 2), with consideration for the magnitude of Cₘₐₓ for each formulation, and the characteristics of the serum testosterone concentration profiles after the point of Cₘₐₓ.

These results indicate that modification of the LPT polymer molecular weight can be used to influence the drug release rate (e.g., use of a higher molecular weight polymer can be used to slow the release rate, lower the Cₘₐₓ (in vivo) or RRₘₐₓ (in vitro), and/or extend the duration of drug release from the formulation, whereas use of a lower molecular weight polymer can be used to increase the release rate, increase the Cₘₐₓ or RRₘₐₓ, and/or shorten the duration of drug release from the formulation). The results additionally showed that both NMP and DMSO solvents, in combination with one of either PEG 300 or PEG 400 as a co-solvent, are suitable solvents for use in an LPT formulation where the active pharmaceutical ingredient (API) is TU or has characteristics similar to those of TU (e.g., has relatively low solubility in aqueous media).

Test Formulations 1-3 as shown in Table 3 were also evaluated for thermal stability, i.e., the ability to remain stable (does not substantially change physical state (i.e., does not undergo a phase transition) in the formulation and remains in suspension or substantially solid form) at body temperatures (e.g., about 36.5° C. to about 37° C.) and up to e-beam temperatures (e.g., 20-45° C.). Thermal stability was evaluated by differential scanning calorimetry (DSC). Briefly, small samples (e.g. about 5-10 mg) of each formulation were sealed in a 40 μL aluminium pan. Samples were slowly heated on a DSC (e.g. a Mettler Toledo TGA/DSC 2), and the temperature at which the drug fully dissolved into solution was determined by identifying the peak temperature of the endothermic dissolution event. FIG. 1C shows the temperature sensitivity (thermal stability) of Test Formulations 1-3, and shows that each formulation must be heated to a temperature greater than 45° C. for the suspended drug to fully dissolve and form a solution. Accordingly each of Test Formulations 1-3 is thermally stable according to the invention, and are denoted as “Homogenized Suspension” (post-e-beam) in Table 3.

All three of the LPT-TU suspension Test Formulations 1, 2 and 3 described above were selected for additional testing in vivo. Briefly, castrated male rats were divided into groups, which were injected with either a control formulation or one of the LPT-TU Test Formulations 1, 2 or 3. The control formulation in this experiment was a non-polymeric solution of testosterone undecanoate formulated in benzyl benzoate and castor oil. Each rat received a single subcutaneous injection of control or test formulation equivalent to 100 mg/kg testosterone undecanoate. Blood samples were collected and processed for measurement of plasma testosterone and testosterone undecanoate concentrations by liquid chromatography/mass spectroscopy (LC/MS) at pre-dose, 30 minutes, 1, 3 and 10 hours on days 1, 4, 7, 14, 21, 28, 35, 42, 56, 70, 91 (all Test Formulations), 112, 125 (Control and Test Formulation 2 only), 140, and 154 (Test Formulation 2 only) days post dose. To evaluate the results, a target range for testosterone release was established between 3 ng/ml and 10 ng/ml, which is approximately equivalent to 10.4-34.7 nmol/L testosterone in plasma and corresponds to testosterone supplementation in the eugonadal range for humans e.g. (see, e.g., Basaria and Morgentaler). Results of this experiment are shown in FIG. 2 (Test Composition 1 (▪), Test Composition 2 (▴), Test Composition 3 (∘), Non-Polymeric TU Control Solution (□)).

As shown in FIG. 2, Test Compositions 1 (▪) and 3 (∘), which were formulated with the lower molecular weight (5 kDa) LPT polymer and either the NMP/PEG 300 or the DMSO/PEG 400 solvent systems, had a Cₘₐₓ above the target range shortly after injection. After Day 21, testosterone levels fell to within the target range (˜3-10 ng/ml) and remained there until about day 91, after which point plasma testosterone was no longer measured in these groups. Test Composition 2 (▴), which was formulated with the 14 kDa LPT polymer and the NMP/PEG 300 solvent system, entered the target range at about Day 10, displaying a lower Cₘₐₓ within the target range, and the mean testosterone concentration remained within the target range until Day 91, and then remained just under the lower target limit for the remainder of the experiment (Day 154). The Non-Polymeric Control Solution of testosterone undecanoate (□) did not enter the target window for the entirety of the experiment.

These results indicate that LPT formulations produced with the higher molecular weight polymer are better candidates for formulations in which longer term release of testosterone undecanoate (and similar drugs) is desired, and additionally have a lower Cₘₐₓ than LPT formulations produced with lower molecular weight polymers. LPT formulations produced with the lower molecular weight polymer may be useful when a more rapid and/or shorter duration of drug release is desired. The results additionally showed that both NMP and DMSO solvents in combination with a PEG co-solvent are suitable solvents for use in an LPT-TU formulation. The NMP/PEG 300 co-solvent system was selected for further studies described herein.

### Example 3

The following example illustrates the effect of polymer molecular weight, testosterone undecanoate particle size, and solvent/co-solvent composition of an LPT-TU formulation on the performance of the formulation.

Experiments were designed to evaluate the effect of three different parameters on the performance of LPT-TU formulations using the NMP/PEG 300 co-solvent system, as measured by in vitro release and TU melt/dissolve temperature.

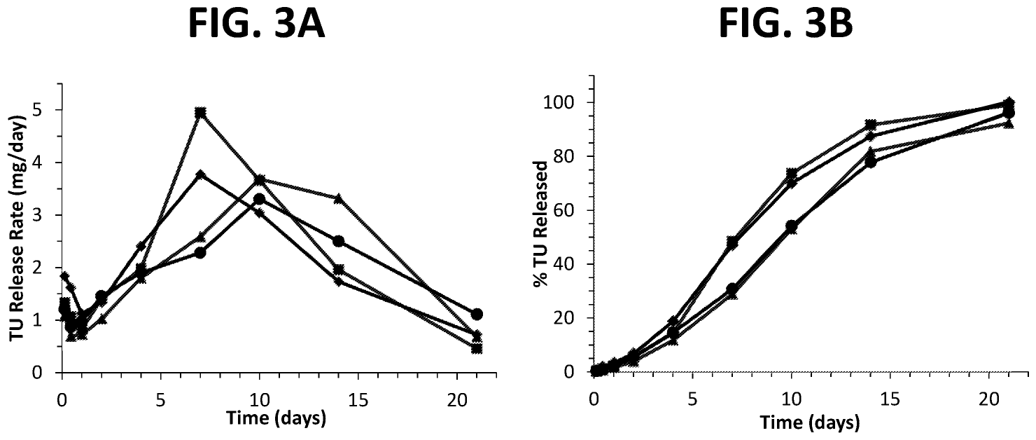

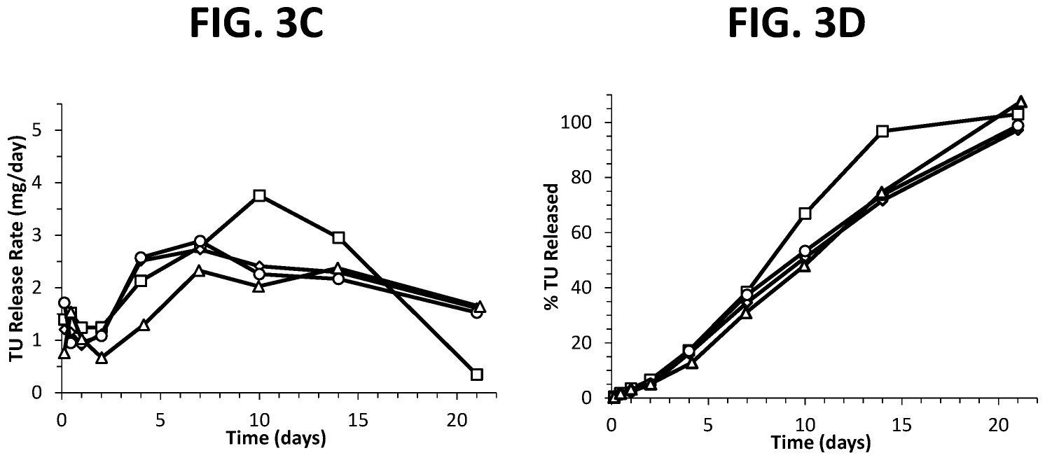

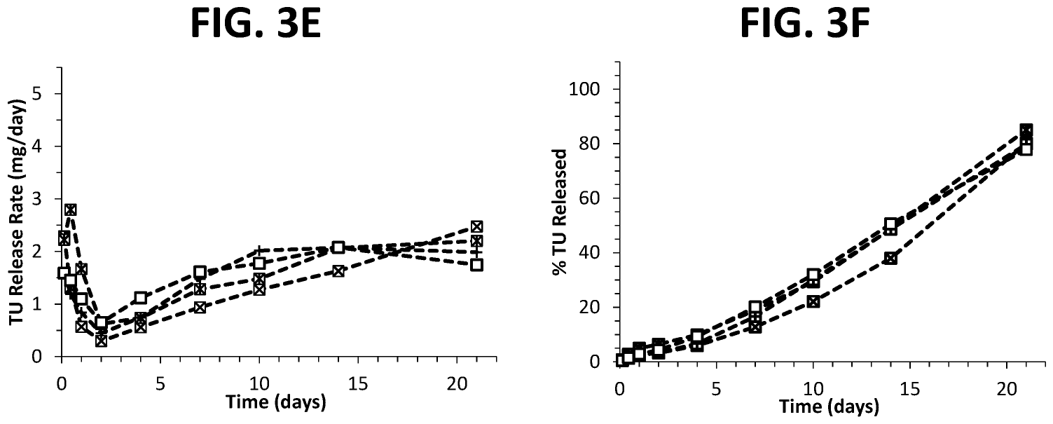

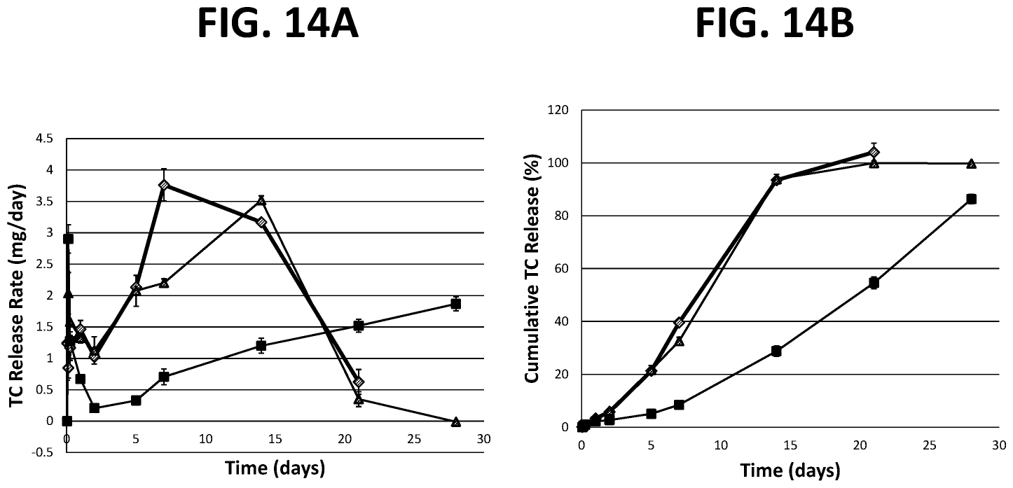

First, since the experiments described in Example 2 showed that LPT polymers of a weight average molecular weight greater than 5 kDa extended the release of drug from the formulation and improved the in vivo kinetics, in order to further demonstrate the effect of polymer molecular weight on the extended release performance of the formulations, LPT polymers having weight average molecular weights of approximately 10 kDa (FIGS. 3A and 3B), 14 kDa (FIGS. 3C and 3D), and 22 kDa (FIGS. 3E and 3F) were prepared. Second, to evaluate the effect of the particle size of TU, LPT-TU formulations were prepared in which the TU was jet milled to achieve a particle size distribution having a D_{v,50} (volume-based particle distribution) of 15 μm, 56 μm, 64 μm, or 90 μm. Finally, to evaluate the effect of the amount of the co-solvent PEG 300 in a formulation comprising the solvent NMP, the amount of PEG 300 was varied in combination with NMP, while maintaining the total amount of solvent in the formulation at the same percentage.

Table 4 shows the LPT-TU formulations used in these experiments. In all formulations, the polymer was a glycolic acid-initiated, 75:25 poly(D,L-Lactide-co-ε-Caprolactone) polymer as described in Example 1, with a molecular weight of 10 kDa, 14 kDa or 22 kDa, present in the formulation at 30% by weight. All formulations used NMP as the solvent and PEG 300 as the co-solvent, where the total amount of solvent (i.e., % NMP+% PEG 300) in the formulation remained constant at 50% by weight of the formulation. Testosterone undecanoate of the indicated particle size (see also Table 2A), was present in all formulations at 20% by weight of the formulation. Test Formulations 9 and 10 are duplicate formulations, produced as separate batches, used to establish reproducibility in the experiment. Test Formulation 11 is is the same as Test Formulation 2 described in Examples 1 and 2, and results are shown here for comparison purposes. The formulations were prepared using the methods described in Example 1, although in this experiment, they were not homogenized nor subjected to e-beam irradiation. The formulations were tested in an in vitro release test, also as described in Example 1.

TABLE 4

| | | | | TU Particle Size | | |
|---|---|---|---|---|---|---|
| LPT-TU | | | | (volume based | | |
| Test | Polymer | % | % | D_{v, 10} | D_{v, 50} | D_{v, 90} |
| Formulation # | MW | NMP | PEG 300 | (μm) | (μm) | (μm) |
| 4 | 10 kDa | 35 | 15 | 2 | 15 | 56 |
| 5 | 10 kDa | 35 | 15 | 9 | 64 | 162 |
| 6 | 10 kDa | 25 | 25 | 7 | 56 | 185 |
| 7 | 10 kDa | 15 | 35 | 2 | 15 | 56 |
| 8 | 14 kDa | 25 | 25 | 2 | 15 | 56 |
| 9 | 14 kDa | 25 | 25 | 7 | 56 | 185 |
| 10 | 14 kDa | 25 | 25 | 7 | 56 | 185 |
| 11 | 14 kDa | 25 | 25 | 16 | 90 | 470 |
| 12 | 22 kDa | 35 | 15 | 2 | 15 | 56 |
| 13 | 22 kDa | 35 | 15 | 9 | 64 | 162 |
| 14 | 22 kDa | 15 | 35 | 2 | 15 | 56 |
| 15 | 22 kDa | 15 | 35 | 9 | 64 | 162 |

FIGS. 3A and 3B show the in vitro TU release rate (FIG. 3A, mg/day) and the percentage TU released over time (FIG. 3B) for the LPT formulations having the 10 kDa polymer (Test Formulation 4 (▪); Test Formulation 5 (▴); Test Formulation 6 (●); Test Formulation 7 (♦)). FIGS. 3C and 3D show the in vitro TU release rate (FIG. 3C, mg/day) and the percentage TU released over time (FIG. 3D) for the LPT formulations having the 14 kDa polymer (Test Formulation 8 (□); Test Formulation 9 (∘); Test Formulation 10 (⋄); Test Formulation 11 (Δ)). FIGS. 3E and 3F show the in vitro TU release rate (FIG. 3E, mg/day) and the percentage TU released over time (FIG. 3F) for the LPT formulations having the 22 kDa polymer (Test Formulation 12 (--X--); Test Formulation 13 (--+--); Test Formulation 14 ( [symbol] ); Test Formulation 15 (--□--)).

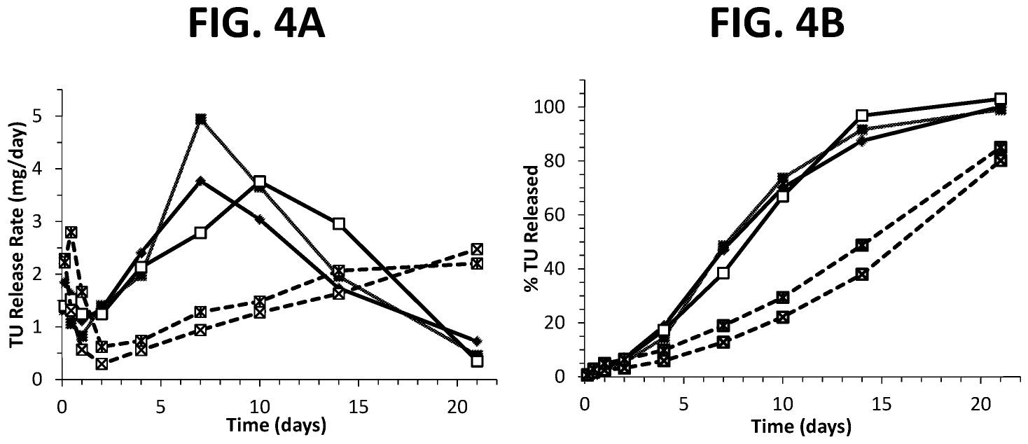

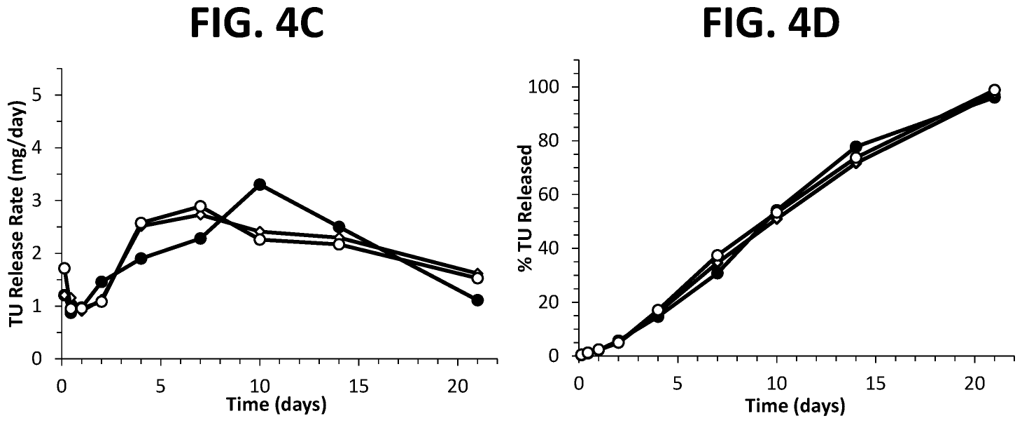

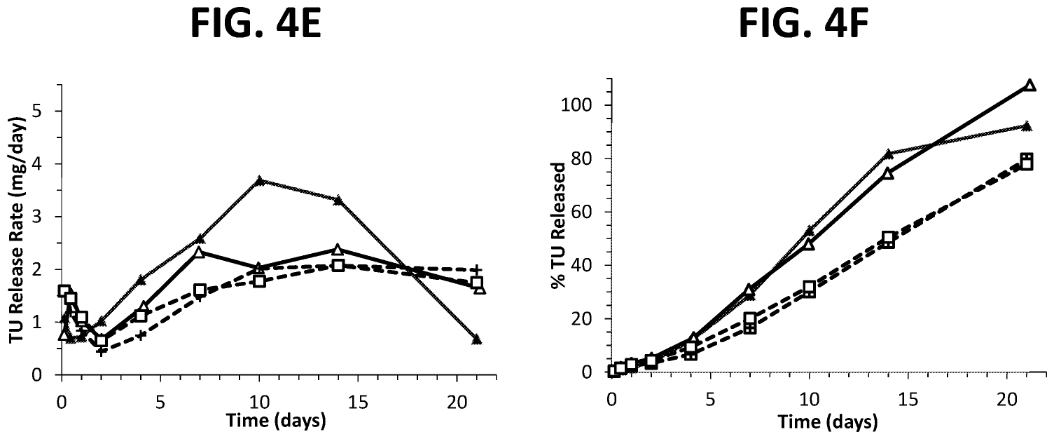

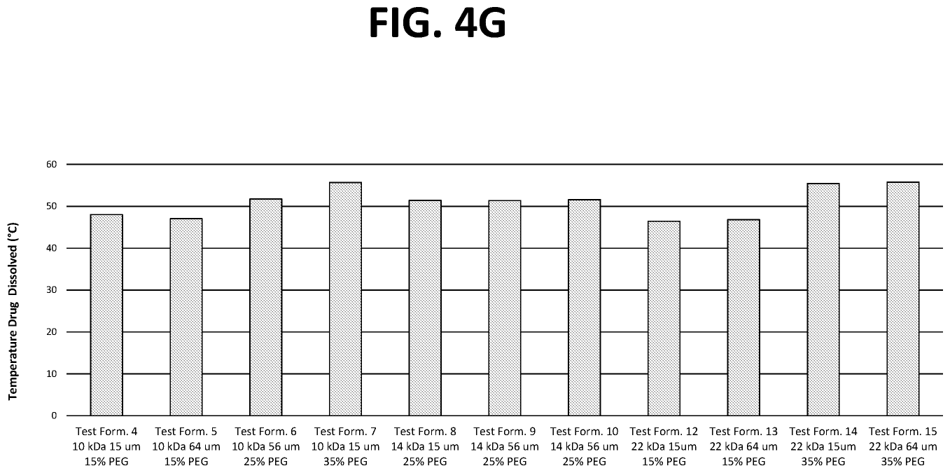

FIGS. 4A-4F present the same data as in FIGS. 3A-3F, but instead the formulations are grouped by particle size (D_{v,50}) instead of by polymer molecular weight. FIGS. 4A and 4B show the in vitro TU release rate (FIG. 4A, mg/day) and the percentage TU released over time (FIG. 4B) for the LPT formulations having 15 μm TU (Test Formulation 4 (▪); Test Formulation 7 (♦); Test Formulation 8 (□); Test Formulation 12 (--X--); and Test Formulation 14 ( [symbol] ). FIGS. 4C and 4D show the in vitro TU release rate (FIG. 4C, mg/day) and the percentage TU released over time (FIG. 4D) for the LPT formulations having 56 μm TU (Test Formulation 6 (●); Test Formulation 9 (∘); and Test Formulation 10 (⋄)). FIGS. 4E and 4F show the in vitro TU release rate (FIG. 4E, mg/day) and the percentage TU released over time (FIG. 4F) for the LPT formulations having 64 or 90 μm TU (Test Formulation 5 (▴); Test Formulation 11 (A); Test Formulation 13 (--+--); and Test Formulation 15--□--).

The results of these experiments demonstrate that the molecular weight of the LPT polymer and the particle size can each be used to control the rate of release of a drug in suspension in the LPT formulations, as well as the duration of release of such a drug from the formulations. More particularly, with respect to molecular weight of the polymer, as shown in FIGS. 3A-3F and FIGS. 4A-4F, the weight average molecular weight of the polymer influences the rate of release of TU from the formulation and the duration of TU release from the formulation. By increasing the weight average molecular weight of the polymer in the LPT formulation, the rate of release of TU can be slowed and the Cₘₐₓ decreased, and the percentage of the release of TU over time is also slowed or extended as the molecular weight increases. For example, comparing FIGS. 3A, 3C and 3E, it can be seen that as the molecular weight of the polymer increases, the release rate curves generally tend to flatten even as the particle size changes, generally lowering the Cₘₐₓ and slowing the TU release rate (the impact of particle size is discussed separately below). This result is perhaps more clearly illustrated by comparing FIGS. 4A, 4C and 4E where in each figure the particle size is held constant. Compositions formulated with the highest molecular weight polymers generally release drug slowly earlier in the experiment, indicating that they may have time delayed initial release of the drug. Comparing FIGS. 3B, 3D and 3F, and again illustrated from the perspective of holding particle size constant in FIGS. 4B, 4D and 4F, as the polymer weight average molecular weight increases, the time in which 50% of the drug is released (T50%) also increases, and the time necessary to release the complete payload of drug will be generally slower

In contrast, as the weight average molecular weight of the polymer decreases,

FIGS. 3A-3F and 4A-4F show that the rate of release of drug from the formulation generally increases (and decreases more rapidly), and higher release rate maxima (RRₘₐₓ) values are produced. As the molecular weight of the polymer within the polymer formulations decreases, the time in which 50% of the drug is released (T50%) also decreases.

Therefore, the desired release rate and duration of release of a drug having the characteristics of TU can be controlled, at least in part, by controlling the molecular weight of the polymer in the LPT formulation. If it is desirable to provide a formulation with a shorter duration of release, where the release occurs more quickly or reaches a higher RRₘₐₓ, then choosing a lower molecular weight polymer is indicated by these experiments, and the inverse is true of the higher molecular weight polymer formulations. While these results demonstrate the effect of polymer molecular weight on in vitro release, similar trends may be expected for in vivo release as well.

As discussed above, the results of these experiments also demonstrate that the particle size (or particle size distribution) also significantly impacts the rate of release of a drug in suspension in the LPT formulations, as well as the duration of release of such a drug from the formulations. More specifically, the results of the experiments shown in FIGS. 3A-3F and FIGS. 4A-4F, showed that increasing the particle size of the drug generally decreased the rate of release of TU and lowered the RRₘₐₓ. Conversely, as the particle size decreased, the TU release rate increased and RRₘₐₓ values were higher and are achieved earlier. Looking more closely at FIGS. 3A, 3C and 3E, where the polymer molecular weight is held constant in each figure, the impact of particle size is evident and is further illustrated in FIGS. 4A, 4C and 4E. The formulations having TU with a D_{v,50} of 15 μm had a higher RRₘₐₓ, a more rapid increase in TU release, which was followed by a more rapid decrease in TU release, thus resulting in the formulations having a shorter duration of release than those formulations that contained the D_{V,50} 56 μm, 64 μm or 90 μm TU as shown in FIGS. 3B, 3D and 3F and in FIGS. 4B, 4D and 4F. In these figures, formulations having TU with a D_{v,50} of 15 μm had a shorter duration of release than the formulations made with larger particle size TU, and vice versa. The effect of particle size on TU release from the formulation was less pronounced in the highest molecular weight formulations (e.g., the 22 kDa formulations), where it appeared that the polymer molecular weight influenced the rate of release and duration of release more than the particle size. The formulations produced with the 14 kDa polymers and TU having a larger TU particle size (56 μm, 64 μm, 90 μm) illustrate the effects of controlling both molecular weight and particle size, since these formulations release TU at a rate that transitions more quickly into the theoretical therapeutic level than the highest molecular weight polymers without the larger RRₘₐₓ “spike” that was observed with the smaller particle sizes and lowest molecular weight polymers, and these formulations also extended the duration of release of drug from the formulation.

Therefore, the desired release rate and duration of release of a drug having the characteristics of TU can be controlled, at least in part, by controlling the particle size of the drug, and by combining the TU particle size control with control of the polymer molecular weight, the drug release rate and duration of release can be further targeted or modified.

With regard to the amount of PEG 300 in the NMP/PEG 300 solvent system, the results of these experiments in FIGS. 3A-3F and FIGS. 4A-4F showed that modification of the amount of PEG 300 in the system did not substantially impact the TU release rate in the formulations, although a more detailed analysis of the results indicated that the higher PEG 300 percentages may be advantageous (data not shown).

Finally, Test Formulations 4-10 and 12-15 as shown in Table 4 were also evaluated for thermal stability, i.e., the ability to remain stable (does not substantially change physical state (i.e., does not undergo a phase transition) in the formulation and remains in suspension or substantially solid form) at body temperatures (e.g., about 36.5° C. to about 37° C.) and up to e-beam temperatures (e.g., 20-45° C.). Thermal stability was evaluated by differential scanning calorimetry (DSC) as described in Example 2. FIG. 4G shows the temperature sensitivity (thermal stability) of Test Formulations 4-10 and 12-15, and shows that each formulation must be heated to a temperature greater than 45° C. for the suspended drug to fully dissolve and form a solution. Accordingly each of Test Formulations 4-10 and 12-15 is thermally stable according to the invention.

Taken together, since the LPT-TU formulations having polymers in the middle of the molecular weight range (e.g., ˜14 kDa) showed the most favorable release kinetics based on release rate, RRₘₐₓ, and duration of release, LPT formulations having polymers with a similar molecular weight were selected for in vivo experiments as described in Example 5, where the impact of TU particle size and the percentage of PEG 300 in the formulation could be further evaluated.

### Example 4

The following example describes the effect of TU particle size on the release rate of LPT-TU formulations in vitro.

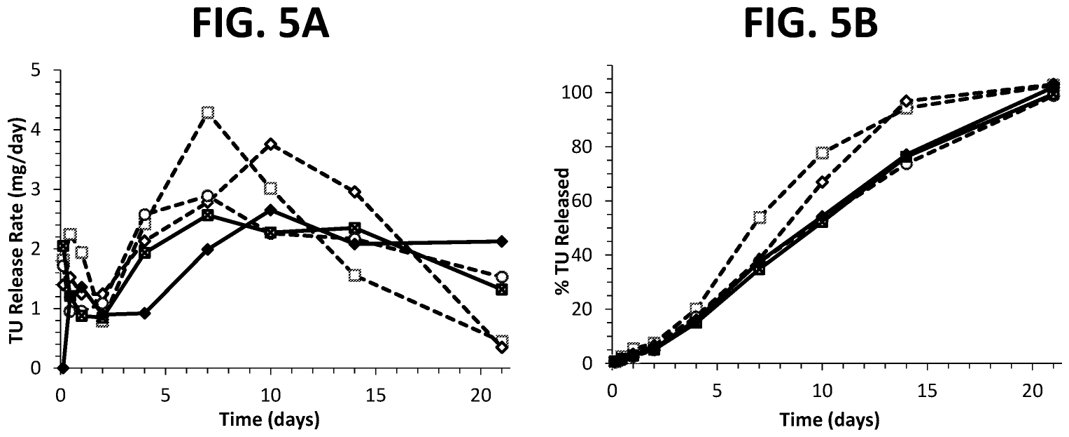

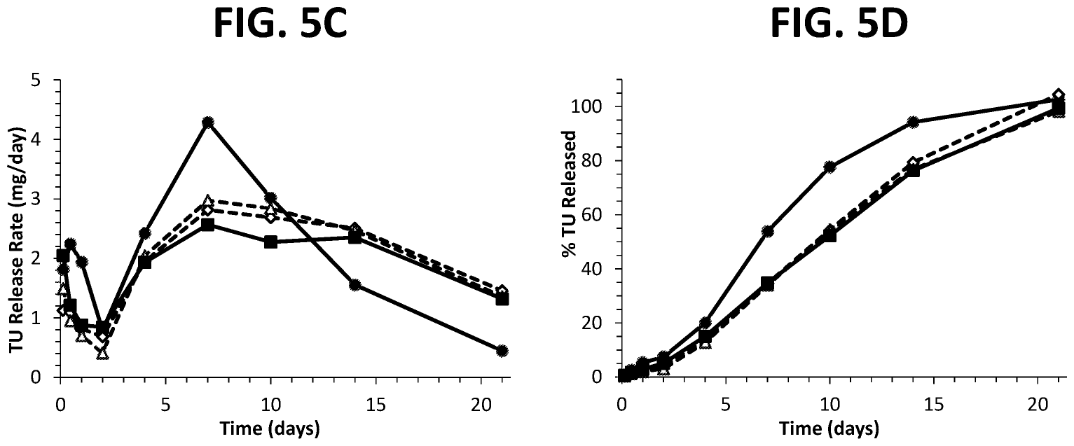

To further evaluate the effect of drug particle size on the drug release rate of LPT suspension formulations, the following experiments were performed. LPT formulations differing only in the particle size of the TU were prepared as follows (see also Table 2A). In all of the formulations, the polymer was a glycolic acid-initiated, 75:25 poly(D,L-Lactide-co-ε-Caprolactone) polymer as described in Example 1, with a molecular weight of ˜14 kDa, present in the formulation at 30% by weight. All formulations contained NMP as the solvent and PEG 300 as the co-solvent, each present at 25% by weight of the formulation (50% total). Testosterone undecanoate (TU) was present in all formulations at 20% by weight of the formulation, with each formulation having a different D_{v,50} particle size as follows: Test Formulation 16 (6 μm TU; FIGS. 5A and 5B, □), Test Formulation 17 (15 μm TU; FIGS. 5A and 5B, ⋄), Test Formulation 18 (56 μm TU; FIGS. 5A and 5B, ∘), Test Formulation 19 (64 μm TU; FIGS. 5A and 5B, X), and Test Formulation 20 (86 μm TU; FIGS. 5A and 5B, ♦).

The TU in these formulations was: (1) wet milled (6 μm TU); (2) jet-milled (15 TU, 56 μm TU, and 64 μm TU) or (3) sieved through a 150 μm sieve (86 μm TU) to achieve the target D_{v,50} particle size as described in Example 1, Table 2A and in Table 5 below. The particle size measurements were calculated using a volume-based particle size distribution method on the drug substance prior to incorporation into the polymer/solvent formulation. The formulations were prepared using the methods described in Example 1, although in this experiment, they were not homogenized nor subjected to e-beam irradiation. The formulations were evaluated in an in vitro release test as described in Example 1.

TABLE 5

| LPT-TU | Polymer | | | TU Particle Size | | |
|---|---|---|---|---|---|---|
| Test | MW | % | % | D_{v, 10} | D_{v, 50} | D_{v, 90} |
| Formulation # | (30 wt %) | NMP | PEG 300 | (μm) | (μm) | (μm) |
| 16 | 14 kDa | 25 | 25 | 1 | 6 | 21 |
| 17 | 14 kDa | 25 | 25 | 2 | 15 | 56 |
| 18 | 14 kDa | 25 | 25 | 7 | 56 | 185 |
| 19 | 14 kDa | 25 | 25 | 9 | 64 | 162 |
| 20 | 14 kDa | 25 | 25 | 19 | 86 | 183 |

FIGS. 5A and 5B show the TU release rate (FIG. 5A) and the percentage TU released over time (FIG. 5B) from each of the formulations in Table 5 in an in vitro release test. FIGS. 5A and 5B show that as the median (D_{v,50}) TU particle size increases, the time in which 50% of the drug is released (T50%) also generally increases, and the duration of release within the target window was extended. The formulations with the smallest particle sizes had the most rapid rate release rates, the highest release rate maxima (RRₘₐₓ), and the shortest duration of release. The experiment also showed that the LPT formulation containing TU having the highest D_{v,50} particle size (86 μm) had a substantially slower initial rate of release than the other formulations, indicating that it could take this formation longer to release a meaningful amount of drug, at least with respect to TU. Therefore, to effectively enter and remain within a therapeutic rate of release for longer periods of time, LPT formulations with TU having a larger D_{v,50}, but where the D_{v,50} particle size is less than 86 μm, may be desirable. For formulations requiring a shorter duration of release, and/or where more rapid spikes in RRₘₐₓ (or referring to the action of formulations in vivo, Cₘₐₓ) or higher RRₘₐₓ (or Cₘₐₓ) values are not an issue, drugs having smaller particle sizes may be desirable.

In order to evaluate the extent to which the inclusion of small drug particles impacts the release rate of the LPT-TU formulations, another experiment was performed using the LPT-TU formulation identical to Test Formulation 19 having a D_{v,50} of 64 μm. TU having a D_{v,50} of 6 μm TU was then titrated into the formulation, and one formulation was prepared using only 6 μm TU and none of the 64 μm TU. Briefly, in this experiment, the polymer was a glycolic acid-initiated, 75:25 poly(D,L-Lactide-co-ε-Caprolactone) polymer as described in Example 1, with a molecular weight of ˜14 kDa, present in the formulation at 30% by weight. The formulations contained NMP as the solvent and PEG 300 as the co-solvent, each present at 25% by weight of the formulation. Testosterone undecanoate (TU) having a D_{v,50} particle size of 64 μm and/or 6 μm was present in all formulations at a total of 20% by weight of the formulation in the following ratios: (1) 6 μm TU (100%) (FIGS. 5C and 5D, ●); (2) 64 μm/6 μm (60%/40%) (FIGS. 5C and 5D, Δ); (3) 64 μm/6 μm (80%/20%) (FIGS. 5C and 5D, ⋄); and (4) 64 μm (100%) (FIGS. 5C and 5D, ▪). The formulations were tested in an in vitro release test as described in Example 1.

FIG. 5C shows the TU release rate (mg/day) and FIG. 5D shows the percentage of TU released over time. Surprisingly, the results show that the titration of 6 μm TU into the 64 μm formulation at levels of 20% or 40% did not have a significant effect on the release profile of 64 μm LPT-TU, although there was a very slight trend toward slowing the initial release rate. These results show that there is little to no effect of the inclusion of small drug particles on the release kinetics of an LPT formulation, when a drug having a larger D_{v,50} is predominant in the formulation. However, without being bound by theory, the inventors believe that in certain LPT formulations, small drug particle size can be used to help to achieve rapid onset of action within the therapeutic range.

### Example 5

The following example describes in vitro and in vivo testing of additional LPT-TU suspension formulations of the invention.

Based on the results of the experiments described in Examples 2-4, additional LPT-TU formulations were designed. These new Test Formulations are described in Table 6 as Test Formulations 21-25; Test Formulation 2 (see Example 2, Table 3) is provided for comparison. In all LPT Test Formulations 21-25, the LPT polymer was a glycolic acid-initiated, 75:25 poly(D,L-Lactide-co-ε-Caprolactone) polymer as described in Example 1, with a molecular weight of between approximately 14 and 15.5 kDa, present in the formulation at 30% by weight. All formulations used NMP as the solvent and PEG 300 as the co-solvent in the amounts indicated in Table 6, to provide a total of 50% by weight solvent in the formulation. Testosterone undecanoate of the indicated D_{v,50} particle size, was present in all formulations at 20% by weight of the formulation. The TU was jet-milled to achieve the indicated target D_{v,50} particle size as described in Example 1. The particle size measurements were calculated using a volume-based particle size distribution method on the drug substance prior to incorporation into the polymer/solvent formulation. The formulations were prepared using the methods described in Example 1, and were homogenized and subjected to e-beam irradiation. A Non-Polymeric Control Solution of testosterone undecanoate (C) as described in Example 1 was also included in the in vivo experiments.

TABLE 6

| | Test | TU Particle Size | | | | | |
|---|---|---|---|---|---|---|---|
| | Formulation | (volume based) | | | LPT | | |
| Test | TU/LPT/Sol/Co-Sol | D_{v, 10} | D_{v, 50} | D_{v, 90} | Polymer MW | | Co- |
| Formulation # | (by weight %) | (μm) | (μm) | (μm) | (kDa) | Solvent | Solvent |
| 2 | 20/30/25/25 | 16 | 90 | 470 | 14 | 25% | 25% PEG |
| | | | | | | NMP | 300 |
| 21 | 20/30/15/35 | 2 | 15 | 56 | 15.5 | 15% | 35% PEG |
| | | | | | | NMP | 300 |
| 22 | 20/30/25/25 | 2 | 15 | 56 | 15.5 | 25% | 25% PEG |
| | | | | | | NMP | 300 |
| 23 | 20/30/25/25 | 7 | 56 | 185 | 14.2 | 25% | 25% PEG |
| | | | | | | NMP | 300 |
| 24 | 20/30/25/25 | 4 | 34 | 173 | 14 | 25% | 25% PEG |
| | | | | | | NMP | 300 |
| 25 | 20/30/25/25 | 12 | 63 | 255 | 14 | 25% | 25% PEG |
| | | | | | | NMP | 300 |
| C | 24/0/48/28 | — | — | — | — | 48% | 28% Castor |
| | | | | | | BzBz | Oil |

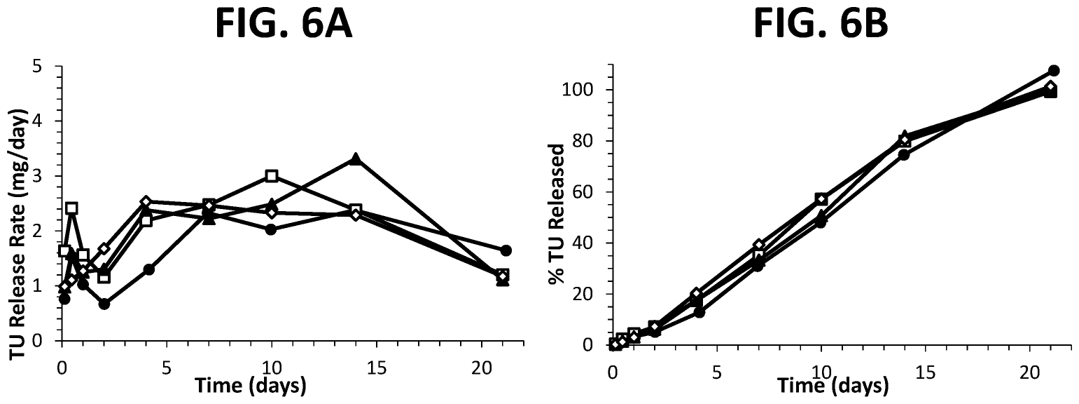

In a first experiment, the results of which are shown in FIGS. 6A and 6B, Test Formulations 21, 22 and 23 were tested in an in vitro release test as described in Example 1. FIG. 6A shows the TU release rate and FIG. 6B shows the percentage TU released over time. Data for Test Formulation 2 is also shown in FIGS. 6A and 6B, but the data is from a different experiment, where the results are overlaid onto FIGS. 6A and 6B for comparison purposes (Test Formulation 2 (FIGS. 6A and 6B, ●), Test Formulation 21 (FIGS. 6A and 6B, □), Test Formulation 22 (FIGS. 6A and 6B, ▴) and Test Formulation 23 (FIGS. 6A and 6B, ⋄)). The results showed that as compared to Test Formulation 2 (●), each of Test Formulations 21, 22 and 23 had a more rapid initial rate of release of TU, thus suggesting a more rapid entry into a therapeutically meaningful rate of release. All four of the formulations released TU for the duration of the experiment. Therefore, each of Test Formulations 21, 22 and 23 were good candidates for testing in vivo.

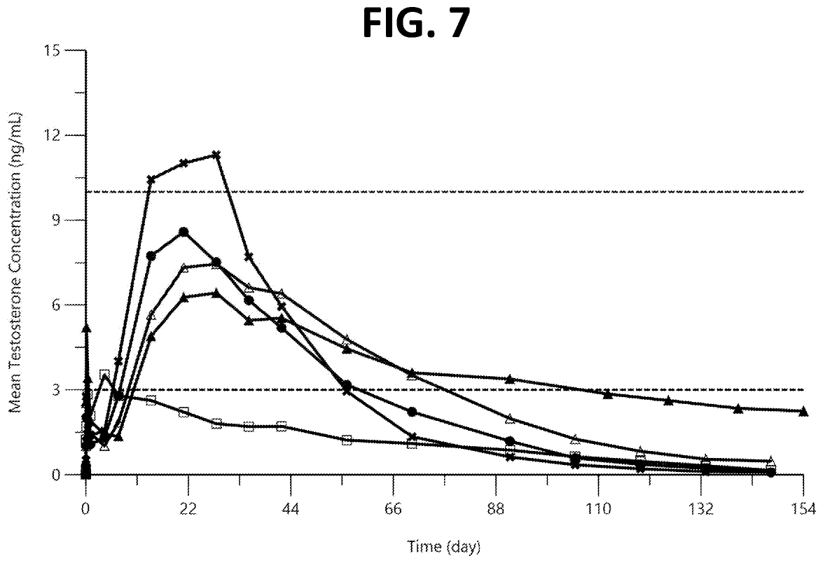

Each of Test Formulations 21, 22 and 23 were tested in in vivo. FIG. 7 shows the results of in vivo testing of the new LPT formulations in rats. Briefly, castrated male rats were divided into groups, which were injected with LPT-TU Test Formulation 21 (FIG. 7, ●), Test Formulation 22 (FIG. 7, X) and Test Formulation 23 (FIG. 7, Δ) described in Table 6. Data from Test Formulation 2 and the experiment described in Example 2 is layered onto this graph for comparison purposes (FIG. 7, ▴). One group of rats received a control formulation (e.g., Non-Polymeric TU Control Solution), which in this experiment, was a non-polymeric solution of testosterone undecanoate formulated in benzyl benzoate and castor oil (FIG. 7, □)). Each rat received a single subcutaneous injection of control or test formulation equivalent to 100 mg/kg testosterone undecanoate. Blood samples were collected and processed for measurement of plasma testosterone and testosterone undecanoate concentrations by liquid chromatography/mass spectroscopy (LC/MS) at pre-dose, 30 minutes, 1, 3, and 10 hours, and on days 1, 4, 7, 14, 20, 28, 35, 42, 56, 70, 91, 105, 119, 133, and 147 post dose.

The results of this experiment showed that all three formulations entered the therapeutic window more quickly than Test Formulation 2. Injection of Test Formulation 22, which contained 15 μm TU and 25% PEG 300, resulted in a Cₘₐₓ and mean testosterone concentration levels just above the target range for several days before returning into the target range. Formulation 23 displayed the longest duration of mean testosterone concentration within the target range among the Test Formulations 21-23, remaining within the target range until almost Day 80. Test Formulations 21 and 22 resulted in testosterone concentrations that dropped below the minimum range prior to Day 60. The overlay of the prior in vivo results from Example 2 using Test Formulation 2 shows that this formulation achieved the longest performance within the target window. Formulations 2, 22, and 23 illustrate an effect of TU particle size on in vivo LPT-TU formulation kinetics, with increased particle size being correlated with later entry of the animals into the target testosterone concentration range, decreased peak plasma testosterone concentration (Cₘₐₓ), and extended duration of formulation activity within the target range. This data demonstrates that particle size can be controlled to affect in vivo formulation kinetics, with smaller particle sizes being advantageous when a higher Cₘₐₓ and shorter duration are desired, while larger particles are advantageous when a lower Cₘₐₓ and longer duration are desired. This data further supports the validity of the trends observed in the in vitro experiments, which also showed that particle size is a useful method to control release from the LPT-TU formulation. It is noteworthy that the in vivo response from the LPT-TU formulations was far more sensitive than the response to the Non-Polymeric Control, given the same (subcutaneous) route of administration, and the same drug dose. Without being bound by theory, it is possible that the LPT-TU delivery system is equipped to provide the desired pharmacokinetic profile in vivo of TU, or an API such as TU, using a reduced drug load, when compared to the Non-Polymeric Control.

### Example 6

The following example describes in vivo studies of LPT-TU suspension formulations in a minipig animal model.

To further evaluate LPT Test Formulations described in Example 5, two of the formulations, LPT Test Formulations 22 and 23, were injected into minipigs and the testosterone release over time was evaluated. Briefly, six castrated male minipigs were divided into two groups which were given Test Formulation 22 or 23. Each minipig received a single subcutaneous injection of approximately 18 mg/kg testosterone undecanoate. Blood samples were collected and processed for measurement of plasma concentrations of testosterone and testosterone undecanoate by liquid chromatography/mass spectroscopy (LC/MS) pre-dose, at 30 minutes, 1 hour, and 3 hours post-dose, and on Days 1, 4, 7, 14, 21, 28, 35, 42, 56, 70, 91, 105, 119, 133, and 147 post injection.

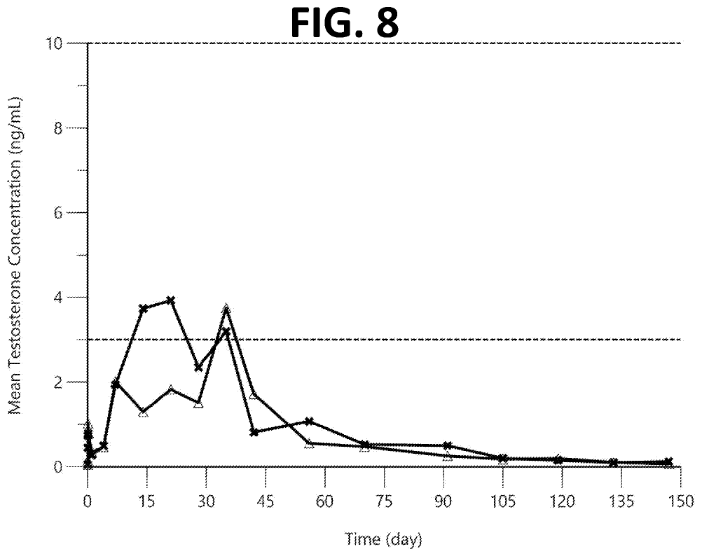

FIG. 8 shows that in this experiment, the duration of testosterone release from both formulations (Test Formulation 22 (X); Test Formulation 23 (A)) was shorter and the mean testosterone concentrations were lower, as compared to the rat experiments shown in Example 5 above, (i.e., the LPT formulations were not able to reach or maintain the testosterone in the target window). Without being bound by theory, the inventors believe that the testosterone dosing of 18 mg/kg was not sufficiently high in the minipig animal species. Therefore, a new experiment evaluated dose escalation of the LPT-TU formulation in minipigs.

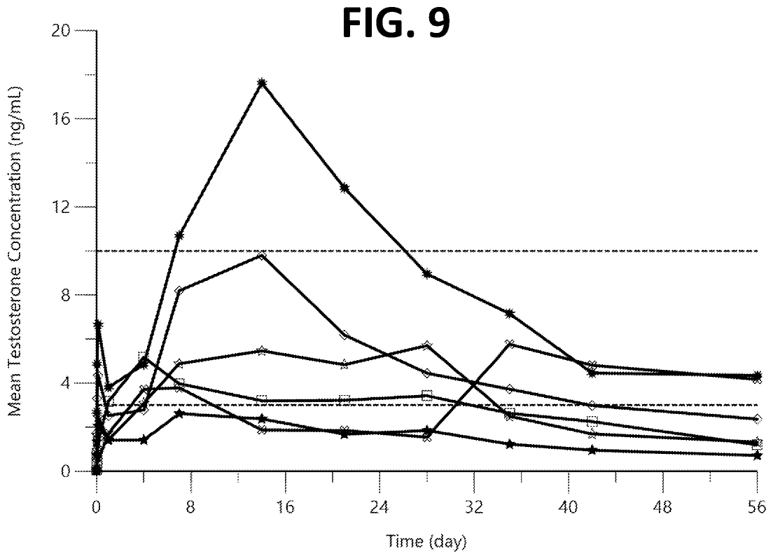

In this new experiment, Controls or LPT Test Formulation 24 were injected into minipigs as follows:
Group A (Control Group, FIG. 9, (□)) received a single intramuscular injection on Day 0 of the Non-Polymeric TU Control Solution (see Example 1) in a dose that delivered approximately 29 mg/kg TU; three of five animals in Group A (Control Group, FIG. 9, (X)) also received a second intramuscular injection on Day 29 of the Non-Polymeric TU Control Solution at 29 mg/kg TU.
Group B (data not shown due to plasma testosterone concentrations below the limit of quantitation) received a single subcutaneous dose (injected at multiple sites due to the volume) of the LPT Polymer-Solvent without testosterone undecanoate (Vehicle Control).
Group C (FIG. 9, (★)) received a single subcutaneous injection on Day 0 of LPT Test Formulation 24 in a dose that delivered approximately 29 mg/kg TU (1.6X dose delivered in the experiment shown in FIG. 8)
Group D (FIG. 9, (⋆)) received a single subcutaneous injection on Day 0 of LPT Test Formulation 24 in a dose that delivered approximately 58 mg/kg TU (3.2X dose delivered in the experiment shown in FIG. 8)
Group E (FIG. 9, (⋄)) received a single subcutaneous dose (injected at multiple sites due to the volume) of LPT Test Formulation 24 in a dose that delivered approximately 116 mg/kg TU (6.4X dose delivered in the experiment shown in FIG. 8); and
Group F (FIG. 9, ( [symbol] )) received a single subcutaneous dose (injected at multiple sites due to the volume) of LPT Test Formulation 24 in a dose that delivered approximately 232 mg/kg TU (12.8X dose delivered in the experiment shown in FIG. 8).

Blood samples were collected and processed for measurement of plasma testosterone and testosterone undecanoate concentrations by liquid chromatography/mass spectroscopy (LC/MS) at pre dose, 30 minutes, 1 hour, 3 hours, and on days 1, 4, 7, 14, 21, 28, 35, 42, 56, 70, 91, 105, 119, 133, and 144 postinjection.

FIG. 9 shows the results of this study at 56 days post-injection. The results show a dose-dependent effect of the LPT-TU formulation. Specifically, as the dosing of TU increased, the mean testosterone concentration entered and remained within or above the target window sooner, the Cₘₐₓ increased, and the duration of the mean testosterone concentration within the target window increased. Group C (★), representing animals receiving the lowest dose of TU, did not achieve mean testosterone concentrations that entered the target window during this experiment. Groups D (⋆), E (⋄) and F ( [symbol] ) all entered the target window between Days 0 and 4, with Groups D and E remaining within the target window until about Days 35 and 42, respectively. Group F had a Cₘₐₓ above the target, and then returned to the target window and remained there through at least Day 56. While plasma concentrations in the LPT-TU treated groups begin to decrease after ˜2-4 weeks depending on the dose, the formulation continues to cause quantifiable increases in plasma testosterone concentrations through at least day 56. With respect to the non-polymeric control group, FIG. 9 shows that in order to achieve mean testosterone concentrations within the target range upon injection of this control, the second dose of the Non-Polymeric TU Control Solution at Day 29 was required (FIG. 9, compare (□) to (x)).

### Example 7

The following example describes in vitro and in vivo studies of LPT-TU solution formulations of the present invention.

Following the preliminary design and screening of over 75 different LPT formulations described in Example 1, LPT-TU formulations, where the TU is in solution in the formulation, were also selected for further evaluation. Specifically, the formulations shown in Table 7 were produced to study the effects of various polymer molecular weights in an LPT-TU formulation that utilizes benzyl benzoate as the solvent, which the inventors discovered solubilizes drugs having the characteristics of testosterone undecanoate, where the formulation is stable at both refrigeration and e-beam temperatures. The second column in Table 7 below lists the composition of the LPT formulation with respect to the percentage by weight of: testosterone undecanoate (TU), LPT polymer (LPT), and solvent (Sol). The third column indicates the LPT polymer and weight average molecular weight (MW) of the polymer. The fourth column identifies the solvent, the fifth column describes the physical form of the testosterone undecanoate in the post-e-beam formulation, and the last column provides the viscosity of the LPT formulation, post-e-beam irradiation. Viscosity of the LPT solutions was tested using a Brookfield rheometer R/S CPS+ with a C50-1 spindle at a sheer rate of 50 or 100 l/s. Each of test formulations A, B, C, D and E has the following characteristics: (1) does not freeze at refrigeration temperatures (-2-8° C.); (2) the formulation is a solution (TU is dissolved in the solvent); and (3) the formulation is easily injected due to lower viscosity. The Control formulation (X) in Table 7 is an LPT-TU suspension formulation (see Example 1) and does not freeze at refrigeration temperatures, but is also not thermally stable at e-beam temperatures (i.e., the TU dissolves at the higher temperatures encountered during e-beam irradiation) and therefore, this formulation recrystallized post-e-beam irradiation.

TABLE 7

| | | | | Formulation | |
|---|---|---|---|---|---|
| Test | Composition | | | Physical | Viscosity |
| Formulation | (TU/LPT/Sol) | Polymer, MW | Solvent | State | (cP) |
| X | 20/30/50 | 75:25 PDLCL, | NMP | Recrystallized | 1576 |
| | | 22 kDa | | Suspension | |
| A | 15/20/65 | 75:25 PDLCL, | Benzyl | Solution | 130 |
| | | 5 kDa | Benzoate | | |
| B | 15/20/65 | 75:25 PDLCL, | Benzyl | Solution | 200 |
| | | 8.5 kDa | Benzoate | | |
| C | 15/20/65 | 75:25 PDLCL, | Benzyl | Solution | 190 |
| | | 10 kDa | Benzoate | | |
| D | 15/20/65 | 75:25 PDLCL, | Benzyl | Solution | 270 |
| | | 14 kDa | Benzoate | | |
| E | 15/20/65 | 75:25 PDLCL, | Benzyl | Solution | 500 |
| | | 22 kDa | Benzoate | | |

To produce the formulations shown in Table 7 above, LPT polymers, which were glycolic acid-initiated, 75:25 DL-lactide/caprolactone (PDLCL) liquid polymers (i.e., polymers comprised of 75% DL-lactide and 25% ε-caprolactone), were produced using the methods described in Example 1, except that the formulations were not homogenized, since they are solutions. Briefly, a 75:25 PDLCL LPT polymer of the indicated weight average molecular weight was combined with the benzyl benzoate solvent, and mixed to assist in the dissolution and/or dispersion of the solvent in the polymer. Complete homogenous dissolution required mixing with a TURBULA® shaker-mixer (GlenMills, N.J.) until visually and tactilely (via interrogation with a metal spatula) homogeneous. The resulting solution was a viscous, but flowable liquid polymer which was at that point a drug-free polymer/solvent composition. Testosterone undecanoate (TU) was added to the polymer solution in the amounts required to achieve the percentages indicated in Table 7 and mixed in a 3-dimensional shaker/mixer or jar mill at 40 rpm for not less than 12 hours until the TU was fully dissolved and a homogeneous solution was formed.

The control sample (X) is designated as a “recrystallized suspension” because the TU in the formulation dissolved into the polymer/solvent matrix during e-beam irradiation temperatures and was then recrystallized, and so this formulation is representative of the LPT-TU formulations which were found to exhibit slower in vivo release as discussed in Example 2.

These LPT solution formulations were tested in an in vitro release assay as described in Example 1. Samples of the in vitro release media were collected at specified timepoints: 3 hours (0.13 days), 11 hours (0.46 days), 1 day, 2 day, 4 day, 7 day, 10 day, 14 day, and 21 day), and each sample was analyzed by HPLC for testosterone undecanoate content. Both the release rate (mg/day) and percent cumulative release (%) of testosterone undecanoate were calculated and reported for each time point.

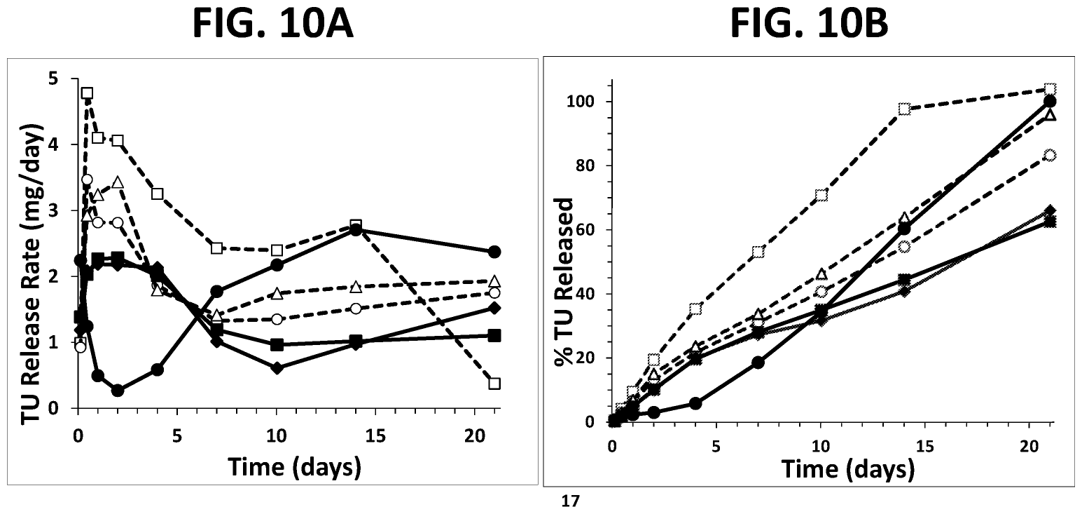

FIGS. 10A and 10B show the TU release rate (FIG. 10 A, mg/day) and the percentage cumulative release of TU (FIG. 10B) from Test Formulations A-E, as compared to the recrystallized suspension Control LPT Formulation (Test Formulation A (□); Test Formulation B (∘); Test Formulation C (Δ); Test Formulation D (♦); Test Formulation E (▪) and Control Suspension (●)). The results showed that the 5 kDa LPT-TU solution (Test Formulation A) reached 100% release of TU in less than 15 days, whereas the LPT-TU solutions having a higher weight average molecular weight (Test Formulations D and E) released TU more slowly, and more similarly to the LPT-TU recrystallized suspension formulation.

FIGS. 10A and 10B show that as the weight average molecular weight of the polymer used in the LPT-TU solution formulations decreases, the formulations release TU much more rapidly early in the experiment and have a higher release rate maxima (RRₘₐₓ) than the formulations made using the higher molecular weight polymers. As the weight average molecular weight of the polymer increases, the results show that the elevated RRₘₐₓ effects diminish, although with increased molecular weight, the exhibit very slow in vitro release of TU, such that the 14 KDa and 22 kDa formulations reached only about 60% TU release by the end of the experiment. Therefore, these results indicate that the TU release rate and duration of release can be modified by changing the weight average molecular weight of the polymer to achieve the desired therapeutic effect, and that for the longest duration of release within the target window, LPT-TU solution formulations having polymer molecular weights in the mid-range (e.g., about 10 kDa to about 14 kDa or slightly higher) are more suitable than those having low molecular weights.

Test Formulation A (5 kDa polymer) was additionally tested in vivo. Briefly, castrated male rats were divided into groups, which were injected with LPT-TU Test

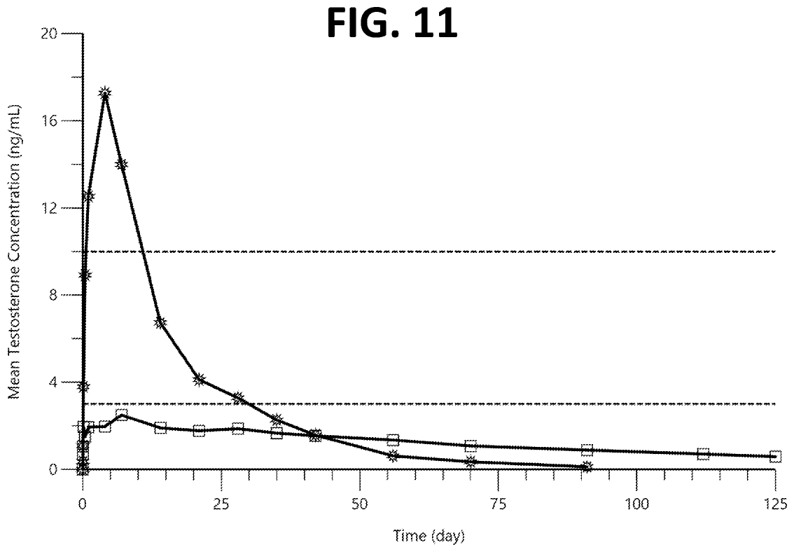

Formulation A or a control formulation (e.g., Non-Polymeric TU Control Solution), which in this experiment, was a non-polymeric solution of testosterone undecanoate formulated in benzyl benzoate and castor oil. Each rat received a single subcutaneous injection of approximately 100 mg/kg testosterone undecanoate. Blood samples were collected and processed for measurement of plasma testosterone concentrations by liquid chromatography/mass spectroscopy (LC/MS) at pre-dose, 30 minutes, 1, 3, and 10 hours, and on days 1, 4, 7, 14, 21, 28, 35, 42, 56, 70, and 91 post injection. To evaluate the results, as in prior experiments described herein, a target range for testosterone release was established between 3 ng/ml and 10 ng/ml, which is approximately equivalent to 10.4-34.7 nmol/L testosterone in plasma and corresponds to testosterone supplementation in the eugonadal range in humans e.g. (see, e.g., Basaria and Morgentaler). Results of this experiment are shown in FIG. 11 (Test Composition A ( [symbol] ), Non-Polymeric TU Control Solution (□)).

FIG. 11 shows that administration of the LPT-TU solution formulation comprising a 5 kDa polymer resulted in a rapid increase in testosterone concentration initially with a high Cₘₐₓ, and the testosterone concentration then rapidly dropped to within the target therapeutic range at about Day 14, remaining in the target range through about Day 29. The Control (Non-Polymeric TU Control Solution) was below the target range for the duration of the experiment. Without being bound by theory, the inventors believed that, based on the in vivo results using the 5 kDa formulation and associated in vitro testing shown in FIGS. 10A and 10B, it would be possible to slow the release of TU from the solution formulation in vivo by increasing the LPT molecular weight above 5 kDa, with the goal of decreasing the Cₘₐₓ and extending the duration of release.

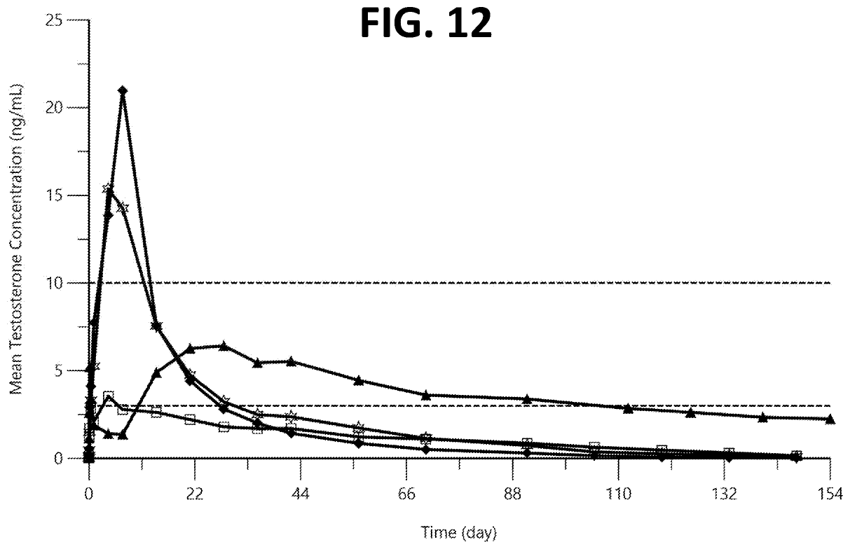

Accordingly, Test Formulations C (10 kDa polymer) and D (14 kDa polymer) were selected for additional testing in vivo. In this experiment, castrated male rats were divided into groups, which were injected with a control formulation or LPT-TU Test Formulation C (FIG. 12, (♦)) or D (FIG. 12, ( [symbol] )), the formulations being described in Table 7. Data from Test Formulation 2 (▴) and the experiment described in Example 2 is layered onto this graph for comparison purposes. One group of rats received the Non-Polymeric TU Control Solution described in Example 1 (i.e., a non-polymeric solution of testosterone undecanoate formulated in benzyl benzoate and castor oil (FIG. 12, □)). Each rat received a single subcutaneous injection of approximately 100 mg/kg testosterone undecanoate. Blood samples were collected and processed for measurement of plasma testosterone and testosterone undecanoate concentrations by liquid chromatography/mass spectroscopy (LC/MS) at pre-dose, 30 minutes, 1, 3, and 10 hours, and on days 1, 4, 7, 14, 20, 28, 35, 42, 56, 70, 91, 105, 119, 133, and 147 post injection.

FIG. 12 shows that both of the LPT-TU solution formulations had a Cₘₐₓ that exceeded the target range in the first two weeks of the experiment, although the Test Formulation D (14 kDa polymer) had a lower Cₘₐₓ, showing that as the polymer molecular weight increases, the Cₘₐₓ can be reduced. Both formulations subsequently entered the target range, remaining there until approximately Day 28. In comparison Test Formulation 2, which is an LPT-TU suspension formulation of the invention, entered the target range later than the solutions, had a much lower Cₘₐₓ, and had a much longer duration within the target window than the solution formulations. These results show that these LPT solution formulations are best utilized with drugs and/or conditions in which a shorter duration of release is desired. In addition, the results show that that polymer molecular weight can be utilized to control the release of the drug from the solution formulation. For some drugs, including those that have physical characteristics in common with TU (e.g., low solubility in aqueous media), it may be desirable to have faster drug release, shorter duration activity, and/or a higher Cₘₐₓ, and these LPT solution formulations provide those features.

### Example 8

The following example provides experimental results directed toward the development of Liquid Polymer Technology (LPT) formulations comprising testosterone cypionate (TC) in the form of a suspension.

To produce an LPT formulation with favorable drug release kinetics and depot degradation, extended release capability (e.g., 60-90 days), and stability within target temperature ranges and time periods, LPT formulations having different molecular weight polymers and/or solvents were produced as described in Example 1 and are shown in Table 8. Table 8: (1) lists the composition of each of the LPT Test Formulations with respect to the percentage by weight of: testosterone cypionate (TC), LPT polymer (LPT), solvent (Sol), and co-solvent (Co-Sol); (2) provides the TC particle size (volume-based, D_{v,50}); (3) indicates the LPT polymer and weight average molecular weight (Polymer, MW) of the polymer; (4) identifies the solvent and co-solvent (if any); (5) describes the physical form of the testosterone cypionate in the (final) formulation; (6) and provides the viscosity of the final LPT formulation.

TABLE 8

| | Test | | | | | | |
|---|---|---|---|---|---|---|---|
| | Formulation | TC Particle Size | | | | Formulation | |
| | TC/LPT/Sol/Co-Sol | (Volume Based | Polymer | | Co- | Physical | Viscosity |
| # | (by weight %) | D_{v, 50}) | (MW) | Solvent | Solvent | State | (cP) |
| 1 | 20/30/25/25 | 29 μm | 75:25 PDLCL | NMP | PEG300 | Suspension | 1383 |
| | | | (10 kDa) | | | | |
| 2 | 20/30/25/25 | 29 μm | 75:25 PDLCL | NMP | PEG 300 | Suspension | 2342 |
| | | | (14 kDa) | | | | |
| 3 | 20/30/25/25 | 41 μm | 75:25 PDLCL | NMP | PEG 300 | Homogenized | 4230 |
| | | | (22 kDa) | | | Suspension | |
| 4 | 20/30/40/10 | 41 μm | 75:25 PDLCL | NMP | PEG 300 | Homogenized | 1538 |
| | | | (22 kDa) | | | Suspension | |
| 5 | 20/30/25/25 | 41 μm | 75:25 PDLCL | NMP | PEG 300 | Homogenized | 8634 |
| | | | (33 kDa) | | | Suspension | |
| 6 | 20/30/40/10 | 41 μm | 75:25 PDLCL | NMP | PEG 300 | Homogenized | 2936 |
| | | | (33 kDa) | | | Suspension | |

PDLCL = poly(DL-lactide-co-ε-caprolactone) liquid polymer
NMP = N-Methyl-2-Pyrrolidone
PEG 300 = Polyethylene glycol, 300 Da

Each of the LPT-TC Test Formulations 1-6 (Table 8) were demonstrated to have the following characteristics: (1) does not freeze at refrigeration temperatures (˜2-8° C.); (2) is thermally stable at e-beam temperatures, or temperatures of 20-45° C. (i.e., TC does not substantially change physical state in the formulation and remains in suspension or substantially solid or suspended form); and (3) the final formulation is a suspension.

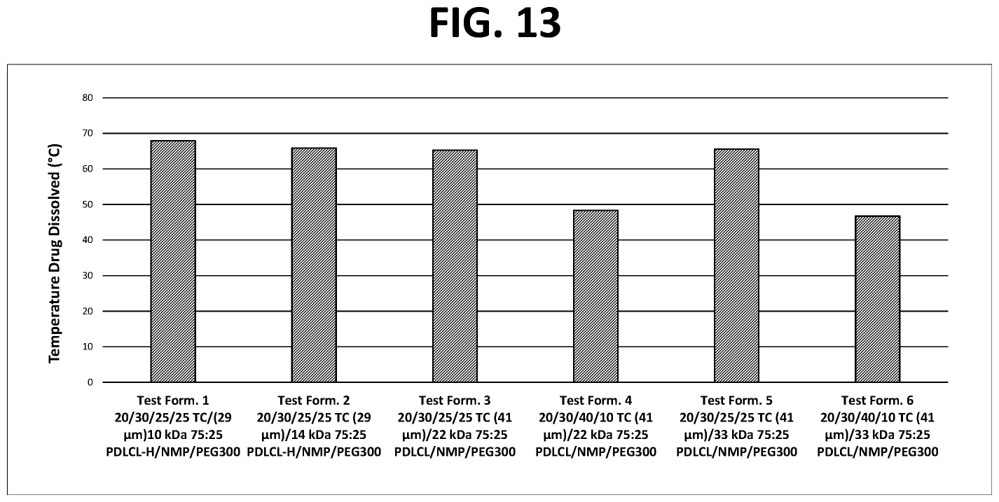

Test Formulations 1-6 as shown in Table 8 were produced as described in Example 1 and were evaluated for thermal stability, i.e., the ability to remain stable (does not change physical state or undergo a phase transition, in the formulation and remains in suspension or substantially solid form) at body temperatures (e.g., about 36.5° C. to about 37° C.) and up to e-beam temperatures (e.g., 20-45° C.). Thermal stability was evaluated by differential scanning calorimetry (DSC). Briefly, small samples (e.g. about 5-10 mg) of each formulation was sealed in a 40 μL aluminium pan. Samples were slowly heated on a DSC (e.g. a Mettler Toledo TGA/DSC 2), and the temperature at which the drug fully dissolved into solution was determined by identifying the peak temperature of the endothermic dissolution event. FIG. 13 shows the temperature sensitivity (thermal stability) of Test Formulations 1-6, and shows that each formulation must be heated to a temperature greater than 45° C. for the suspended drug to fully dissolve and form a solution.

Viscosity was assessed using a cone and plate rheometer (e.g. a Brookfield R/S CPS+) with spindle (e.g. C50-1) at various shear rates; typically viscosity at 100 s¹ is reported. Each of Test Formulations 1, 2, 3, 4 and 6 have viscosities under 5000 cP, making them suitable for injection using a 20 G needle. Test Formulation 5 has the highest viscosity at 8634 cP, which higher than generally desired for a formulation of the present invention when using a 20 G needle, unless a larger needle is used. Also, a comparison of Test Formulation 5 with Test Formulation 6, which differ only in the amount of PEG 300 in the formulation, shows that the viscosity of the formulation can be easily adjusted to a more suitable range simply by modifying the co-solvent content, while not sacrificing thermal stability.

Test Formulations 1, 2 and 4 as shown in Table 8 were selected for further evaluation in an in vitro TC release test also as described in Example 1. FIGS. 14A and 14B show the results of the in vitro release testing of Test Formulations 1, 2 and 4 (Test Formulations 1 (♦), 2 (▴), and 4 (▪). FIG. 14A shows the TC release rate (mg/day) and FIG. 14B shows the percentage of TC (cumulative) released over time (days).

FIGS. 14A and 14B show that Test Formulations 1 and 2 release TC in a similar manner and relatively quickly, whereas Test Formulation 4 releases TC more slowly than either of the other two formulations. FIG. 14B shows that Test Formulations 1 (10 kDa polymer) and 2 (14 kDa polymer) achieved 100% release by approximately Day 21 of the experiment, whereas the Test Formulation 4, comprising the higher molecular weight polymer (22 kDa), released the drug more slowly and was at about 55% release by Day 21 and 86% release by Day 28. FIG. 14A is an alternative representation of in vitro release data, where the rate of TC release is plotted against time. FIG. 14A shows that the maximum in vitro release rate (“RRₘₐₓ”) is much higher for Test Formulations 1 and 2 than for Test Formulation 4. As discussed previously herein, reference to RRₘₐₓ, or maximum release rate, in an in vitro test, refers to the maximum (peak) in vitro release rate from the formulation. A similar measurement is used in in vivo tests, which is called Cₘₐₓ. Reference to “Cₘₐₓ” typically refers to a pharmacokinetic measurement of rate that is the maximum (peak) serum concentration of the drug achieved after a dose of the drug is given, and is typically used in in vivo studies.

One can also review in vitro data such as that shown in FIGS. 14A and 14B and elsewhere herein by referring to the T50%, which is the time it takes to achieve 50% drug release (e.g., with reference to FIG. 14B, one can calculate the time (day) at which 50% of drug was released, which is a useful additional comparison, particularly when one formulation releases drug much more quickly and reaches 100% release much earlier, as compared to a slower releasing formulation. FIG. 14B shows that Test Formulations 1 and 2 reach T50% much earlier than Test Formulation 4.

These results again show that modification of the LPT polymer molecular weight can be used to influence the drug release rate (e.g., use of a higher molecular weight polymer can be used to slow the release rate, lower the Cₘₐₓ (in vivo) or RRₘₐₓ (in vitro), and/or extend the duration of drug release from the formulation, whereas use of a lower molecular weight polymer can be used to increase the release rate, increase the Cₘₐₓ or RRₘₐₓ, and/or shorten the duration of drug release from the formulation). The results additionally showed that NMP, in combination with PEG 300 as a co-solvent, is a suitable solvent for use in an LPT formulation where the active pharmaceutical ingredient (API) is TC, which is an API with characteristics similar to those of TU (e.g., has relatively low solubility in aqueous media).

Various modifications of the above described invention will be evident to those skilled in the art. It is intended that such modifications are included within the scope of the following claims.

1.-82. (canceled)

83. A pharmaceutical composition, comprising:
an active pharmaceutical ingredient having a log P of at least about 0;
a biocompatible solvent or combination or mixture of solvents and/or co-solvents; and
a biodegradable liquid polymer,
wherein the active pharmaceutical ingredient is in a substantially solid form in the biodegradable liquid polymer and the biocompatible solvent or combination or mixture of solvents and/or co-solvents at body temperature, and
wherein the active pharmaceutical ingredient has a D_{v,50} of between about 1 μm and about 250 μm and a particle size span of between about 1 and about 8.

84. The pharmaceutical composition of claim 83, wherein the active pharmaceutical ingredient is in a substantially solid form in the biodegradable liquid polymer and the biocompatible solvent or combination or mixture of solvents and/or co-solvents at temperatures up to between about body temperature to at least about 45° C.

85. The pharmaceutical composition of claim 83, wherein the active pharmaceutical ingredient is a hydrophobic small molecule organic compound having a log P of at least about 2.5 or a pharmaceutically acceptable salt, ester, hydrate, solvate, or prodrug thereof.

86. The pharmaceutical composition of claim 83, wherein the active pharmaceutical ingredient has a D_{v,50} of between about 15 μm and about 200 μm.

87. The pharmaceutical composition of claim 83, wherein the active pharmaceutical ingredient has a D_{v,50} of between about 50 μm and about 150 μm.

88. The pharmaceutical composition of claim 83, wherein the active pharmaceutical ingredient has a particle size span of between about 2 and about 6.

89. The pharmaceutical composition of claim 83, wherein the biocompatible solvent or combination or mixture of solvents and/or co-solvents is selected from the group consisting of acetone, butyrolactone, ε-caprolactone, N-cycylohexyl-2-pyrrolidone, diethylene glycol monomethyl ether, dimethyl acetamide, dimethyl formamide, dimethyl sulfoxide (DMSO), ethyl acetate, ethyl lactate, N-ethyl-2-pyrrolidone, glycerol formal, glycofurol, N-hydroxyethyl-2-pyrrolidone, isopropylidene glycerol, lactic acid, methoxypolyethylene glycol, methoxypropylene glycol, methyl acetate, methyl ethyl ketone, methyl lactate, N-methyl-2-pyrrolidone (NMP), low-molecular weight (MW) polyethylene glycol (PEG), polysorbate 80, polysorbate 60, polysorbate 40, polysorbate 20, polyoxyl 35 hydrogenated castor oil, polyoxyl 40 hydrogenated castor oil, sorbitan monolaurate, sorbitan monostearate, sorbitan monooleate, benzyl alcohol, n-propanol, isopropanol, tert-butanol, propylene glycol, 2-pyrrolidone, α-tocopherol, triacetin, tributyl citrate, acetyl tributyl citrate, acetyl triethyl citrate, triethyl citrate, esters thereof, and combinations and mixtures thereof.

90. The pharmaceutical composition of claim 83, wherein the biocompatible solvent or combination or mixture of solvents and/or co-solvents comprises a biocompatible solvent in combination with a low-molecular weight (MW) polyethylene glycol (PEG).

91. The pharmaceutical composition of claim 83, wherein the biocompatible solvent or combination or mixture of solvents and/or co-solvents is selected from the group consisting of: dimethyl sulfoxide (DMSO), N-methyl-2-pyrrolidone (NMP), a low-molecular weight (MW) polyethylene glycol (PEG), and combinations and mixtures thereof.

92. The pharmaceutical composition of claim 91, wherein a ratio of PEG to NMP or DMSO is between about 20:80 and about 80:20.

93. The pharmaceutical composition of claim 83, wherein the biodegradable liquid polymer comprises lactide residues and one or both of ε-caprolactone residues and trimethylene carbonate residues.

94. The pharmaceutical composition of claim 83, the biodegradable liquid polymer comprises lactide and ε-caprolactone residues and has a ratio of lactide to ε-caprolactone residues from 60:40 to 90:10.

95. The pharmaceutical composition of claim 83, wherein the biodegradable liquid polymer is formed with a hydroxy acid initiator.

96. The pharmaceutical composition of claim 83, wherein the biodegradable liquid polymer has a weight average molecular weight of between about 1 kDa and about 35 kDa.

97. The pharmaceutical composition of claim 83, wherein the active pharmaceutical ingredient makes up about 20 wt % of the pharmaceutical composition, the biocompatible solvent or combination or mixture of solvents and/or co-solvents makes up about 50 wt % of the pharmaceutical composition, and the biodegradable liquid polymer makes up about 30 wt % of the pharmaceutical composition.

98. The pharmaceutical composition of claim 83, wherein the pharmaceutical composition forms a liquid depot in a subject upon injection and the liquid depot releases the active pharmaceutical ingredient into the subject for a period of at least about 30 days.

99. A pharmaceutical composition, comprising:
an active pharmaceutical ingredient having a log P of at least about 0;
a biocompatible solvent or combination or mixture of solvents and/or co-solvents; and
a biodegradable liquid polymer comprising lactide and ε-caprolactone residues and having a weight average molecular weight of about 1 kDa-about 35 kDa, wherein a ratio of lactide to ε-caprolactone residues is from 60:40-90:10,
wherein the active pharmaceutical ingredient is in substantially solid form in the biodegradable liquid polymer and the biocompatible solvent or combination or mixture of solvents and/or co-solvents at body temperature, and
wherein the active pharmaceutical ingredient has a D_{v,50} of about 1 μm-about 250 μm and a particle size span of about 1-about 8.

100. The pharmaceutical composition of claim 99, wherein the active pharmaceutical ingredient is a hydrophobic small molecule organic compound or a pharmaceutically acceptable salt, ester, hydrate, solvate, or prodrug thereof the active pharmaceutical and has a log P of at least about 2.5.

101. The pharmaceutical composition of claim 99, wherein the biocompatible solvent or combination or mixture of solvents and/or co-solvents comprises a biocompatible solvent in combination with a low-molecular weight (MW) polyethylene glycol (PEG).

102. A method of treating a subject, comprising injecting a pharmaceutical composition into the body to the subject, wherein the pharmaceutical composition comprises:
an active pharmaceutical ingredient having a log P of at least about 0;
a biocompatible solvent or combination or mixture of solvents and/or co-solvents;
and
a biodegradable liquid polymer,
wherein the active pharmaceutical ingredient is in substantially solid form in the biodegradable liquid polymer and the biocompatible solvent or combination or mixture of solvents and/or co-solvents at body temperature,
wherein the active pharmaceutical ingredient has a D_{v,50} of between about 1 μm and about 250 μm and a particle size span of between about 1 and about 8, and
wherein the pharmaceutical composition forms a liquid depot in the body of the subject upon injection and the liquid depot releases the active pharmaceutical ingredient into the subject.
